(19) United States
(12) Patent Application Publication
(10) Pub. No.: US 2018/0272121 A1
(43) Pub. Date: Sep. 27, 2018

(54) CARDIAC STIMULATION OF ATRIAL-VENTRICLE PATHWAYS AND/OR ASSOCIATED TISSUE

(71) Applicant: THE MEDICAL RESEARCH, INFRASTRUCTURE AND HEALTH SERVICES FUND OF THE TEL AVIV MEDICAL, Tel-Aviv (IL)

(72) Inventor: Lior YANKELSON, Tel-Aviv (IL)

(21) Appl. No.: 15/541,778

(22) PCT Filed: Jan. 7, 2016
(86) PCT No.: PCT/IL2016/050024

§ 371 Date: Jul. 6, 2017

Related U.S. Application Data
(60) Provisional application No. 62/100,928, filed on Jan. 8, 2015.

(51) Int. Cl. A61N 1/05; A61N 1/362; A61N 1/365

(52) U.S. Cl. CPC A61N 1/057; A61N 1/365; A61N 1/0573; A61N 1/3628

(57) ABSTRACT

The present disclosure provides, according to some embodiments, methods and systems for treatment or prevention of ventricular symptoms of atrial fibrillation. For example, methods for applying sub-threshold electric fields to Av node and associated tissue, for example, from inside a coronary sinus.

### RELATED APPLICATION/S

This application claims the benefit of priority and under 35 USC § 119(e) of U.S. Provisional Patent Application No. 62/100,928, by same inventor, filed Jan. 8, 2015, the contents of which are incorporated herein by reference in their entirety.

### FIELD AND BACKGROUND OF THE INVENTION

The present invention, in some embodiments thereof, relates to applying an electric field to the heart and, more particularly, but not exclusively, to a method and system for affecting signal conduction pathways between the atria and ventricles, for example, for treating the symptoms of atrial fibrillation.

Following is a description of a typical, healthy electrical activation of the heart. Normal cardiac sinus rhythm is mediated by electrical impulse that is created by heart myocytes. The initial impulse originates in the sinoatrial (SA) node, or sinus node, which is located in the upper right atrium of the heart and serves as the primary pacemaker. The electrical impulse spreads from the SA node to the right and left atria through interatrial tracts, thus causing depolarization of atrial cell membranes, which corresponds to a P-wave in an electrocardiogram (ECG or EKG).

Following atrial depolarization, the electrical impulse spreads to the atrioventricular node (AV node). The AV node is generally recognized to be a compact area (˜1×3×5 mm) of specialized tissue in the posteroinferior region of the interatrial septum, near the opening of the coronary sinus, which conducts the normal electrical impulse from the atria to the ventricles. The AV node has its own pacing rhythm that serves as a backup pacemaker in case the SA node fails to initiate an electrical impulse. Consequently, the AV node slows down the electrical impulse by approximately 0.12 seconds, thus ensuring that the atria have ejected their blood into the ventricles prior to ventricle contraction. The AV node's normal intrinsic firing rate without stimulation (such as that from the SA node) is 40-60 times/minute.

From the AV node, the electrical impulse travels through the bundle of His, which bifurcates into the left and right bundle branches. From the branches, the impulse travels through the Purkinje fibers and allows the electrical impulse to end in the ventricles to initiate ventricular depolarization which corresponds to the QRS wave in an electrocardiogram.

Atrial fibrillation (also termed “AF” or “A-Fib”) is the most common cardiac arrhythmia, characterized by disruption of normal electrical impulses generated by the sinoatrial node (SA node) due to uncoordinated atrial electrical impulses. The loss of coordinated atrial contraction in AF, often originating at the roots of the pulmonary veins, leads to irregular conduction of electrical signals to the ventricles. As a result, AF leads to various outcomes such as tachycardia, shorter diastolic fill time, reduced coronary circulation, ischemia, cardiomyopathy etc. Therefore, atrial fibrillation is a source of significant morbidity and mortality.

Atrial fibrillation which is manifested by recurring episodes lasting from minutes to days is termed paroxysomal. Atrial fibrillation manifested by episodes lasting more than 7 days is termed persistent. Treatment of AF may include medications, electrical cardioversion and, in some cases, surgical or catheter-based ablation to either slow the heart rate to a normal range (“rate control”) and/or revert the heart rhythm to a normal sinus rhythm (“rhythm control”). Exemplary treatment is using anti-arrhythmic agents which prolong the effective refractory period (ERP) in which a new action potential cannot be initiated in a tissue once it has been depolarized. Such arrhythmic drugs, however, affect both the atria and ventricles and thus may induce other types of arrhythmias.

Atrial fibrillation is typically manifested by episodes which last long periods of time (such as a year) and which is un-responsive to treatment is termed permanent/chronic AF or refractory AF. In cases of chronic AF, treatment using rhythm control is not feasible and thus the most common treatment is to use a rate control strategy using anti arrhythmic drugs. Unfortunately, all medications that decrease ventricular rate are also able to decrease inotropy and thus decrease cardiac output. Moreover, systemic pharmacologic treatment carries a significant risk for side effects and adverse effects, precluding its use in many patients. Thus, the use of a pacemaker to control heart rate is often required in patients with poorly controlled AF-induced tachycardia. Use of a pacemaker in refractory AF patients requires an irreversible ablation of the AV node and surgical insertion of a pacemaker (“ablate and pace”). Recent studies have shown that although about 10% of atrial fibrillation patients require a pacemaker, only 3% actually undergo the surgical procedure due to its irreversibility and the need to replace the pace maker every 5-10 years.

Atrial flutter is an arrhythmia characterized by rapid and regular rhythm, usually derived from reentry in the right and or left atria. When the arrhythmia is so-called, “typical”, it is amendable for RF ablation. In recent years there has been a tremendous increase of non-typical atrial flutter, as well as atrial tachycardia. These arrhythmias are less organized and many times can not be adequately treated with ablation.

Atrial tachycardia is an arrhythmia characterized by a very rapid regular contraction rate, caused by rapid contraction of the atria.

A study by Mazgalev et al. disclosed application of sub-threshold current (lower than the threshold of myocardial excitation) at AV nodes of isolated rabbit hearts in which AF was simulated by random high right atrial pacing (Mazgalev et al., Circulation, 1999, 99(21):2806-14). Mazgalev et al. concluded that post ganglionic vagal stimulation applied during AF could produce ventricular rate slowing.

U.S. Pat. No. 6,256,537 discloses a system for regulating ventricular rate in the presence of abnormally high atrial rates, such as during episodes of atrial fibrillation. During such episodes the system applies sub-threshold bursts of stimulus pulses to or proximate to the patient's AV node so as to inhibit conduction of electrical signals through to the ventricle during the bursts.

US 2008/0119911 discloses an implantable intravascular device that has a stent-like structure for intravascular fixation. The disclosed device includes embedded microcircuits to allow bipolar and unipolar sensing of cardiac and neurologic electrical activity, sensing of other physiologic signals and local electrical stimulation (cardiac pacing and defibrillation; neurologic stimulation of brain and specific nerve sites and seizure therapy).

U.S. Pat. No. 6,397,109 discloses a single introduction electro-catheter to be used for permanent, semi-permanent or temporary cardiac stimulation through the Coronary Sinus. The foregoing examples of the related art are intended to be illustrative and not exclusive.

### SUMMARY OF THE INVENTION

An aspect of some embodiments of the present invention relates to application of an electric field from within the coronary sinus to affect signal propagation between the atria and ventricles. In some embodiments the applied electric field interferes with the propagation of signals from the atria to the ventricles, allowing only selective signals to reach the ventricles. In some embodiments electric field application allows to increase the number of signals arriving to the ventricles. In some embodiments the applied electric field is a sub-threshold electric field, which is below the threshold required to induce an action potential or to cause cell contraction. In some embodiments, application of a sub-threshold electric field is combined with application of an electric field which is supra-threshold. A supra-threshold electric field is an electric field which is higher than the threshold necessary to induce an action potential or to cause cell contraction. In some embodiments the sub-threshold and/or supra-threshold electric fields are applied as a continuous burst, a single burst, a multiple-discrete burst or a combination thereof.

The inventor has surprisingly realized that electric field delivery to the AV node and associated tissue may be beneficially achieved from within the CS, and, for example, locations near the CS ostium. In particular this has a potential advantage of stable anchoring of the stimulation electrodes without barb-type attachment to the AV node and/or atrial tissue. Atrial tissue is generally thin and less conducive to attachment than ventricular tissue. A further synergy may be provided in some embodiments in that while attachment is secure, precision in electrode placement is provided after securing, for example, by allowing the selection of one or more of several electrodes. This may allow securing to be separate from positioning. A further possible synergy of some embodiments of the invention is that undesired electrification by sub-threshold signals, for example high-power sub-threshold bursts may be reduced. Optionally, the location and/or power of electrode are selected such that a reduced amount of non-target tissue is affected. This may be especially important for sub-threshold signals, which can be strong enough to affect tissues that are not the target tissue. By reducing the power and/or selecting an electrode which affects substantially only tissue of interest and/or less non-target tissues, such side effects may be avoided or reduced. A further possible synergy is that other tissue associated with the AV node, for example, AV node extensions (e.g., rightward and leftward posterior extensions) and/or other tissues of interest within Koch's triangle, may be accessible from within the CS. In some exemplary embodiments of the invention, the extensions stimulated are as described in “Posterior Extensions of the Human Compact Atrioventricular Node; A Neglected Anatomic Feature of Potential Clinical Significance” by Shin Inoue, and Anton E. Becker, Circulation 1998; 97: 188-193 doi: 10.1161/01.CIR.97.2.188. In some exemplary embodiments of the invention, stimulation is directionally applied so that it preferentially extends from the coronary sinus and coronary sinus ostium towards an expected anatomical location of such extensions. For example, underneath or slightly anterior to (e.g., 0-4 mm, for example, 1-3 mm) and below the anterior margin of the mouth of the CS. As noted in said article, not all patients have both extensions. Optionally, mapping (e.g., as described herein) is used to determine the existence of such extensions in the patient. In some embodiments mapping is performed by an electrophysiological study and/or by pacing maneuvers. Optionally or alternatively, mapping is performed by the disclosed system, through assessment of various electrode combinations to determine an effective inhibitory stimulation.

In some exemplary embodiments of the invention, the electric filed directly interacts with a tissue of interest, for example, an AV node, with the electric field acting on a muscle fiber modifying the state and future and/or present behavior of the interacted with muscle fiber.

In some embodiments the applied electric field indirectly affects signal conduction through the AV node region, for example by affecting inputs into the AV node, which then modify AV activity, for example, by directly affecting muscle tissue at the vicinity of the AV node (e.g., one or more AV node extensions) which then affects the AV node itself, for example, acting as controlling input thereto. In some embodiments, electric field can be delivered to neural tissue and/or a fatpad in addition to or instead of directly affecting muscle tissue to achieve the desired effect on the AV node. Typically, the AV node region is located in the lower portion of the right atrium near the right ventricle, at the apex of the triangle of Koch, which is an anatomically triangular region on the septal wall of the right atrium demarcated by the tendon of Todaro, septal leaflet of the tricuspid valve, and the orifice of the coronary sinus. The coronary sinus collects the majority of venous blood from the myocardium. It enters the right atrium through an opening called the coronary sinus ostium, which has a diameter (in adults) of typically 5-15 mm. The coronary sinus has a length (in adults) of typically 3-10 cm, and a typical distal diameter of 1-10 mm (e.g., in adults). Optionally, the CS has a convergence angle (which may vary along its length) of between 5 and 50 degrees, for example, between 10 and 30 degrees, which may affect the design of various components as described herein which may be designed to conform or approximate or somewhat deform the CS. While the CS is a blood vessel it may be weaker than an artery and may be able to tolerate less force, for example, a radial force of less than 20N, 10N, 5N, 3N or intermediate forces may be desired (e.g., in a self-expanding electrode anchoring configuration.

In some embodiments, an electric field is applied directly and/or indirectly to AV node input tissues, for example left inferior extension and/or right inferior extension of the AV node. The left and right inferior extensions of the AV node conduct signals from the left and right atria to the AV node. In some embodiments electric field is applied according to application protocols. In some embodiments application protocols include information about frequency, current and timing parameters of the electric field. In some embodiments the electric field is transferred through the CS wall.

In some embodiments of the invention, the device and/or method provide transient, controlled, AV node blocking, optionally without the use of medicine. In some embodiments, AV blocking medicines and/or other anti-arrhythmia medicine may be provided concurrently.

In some exemplary embodiments of the invention, conduction of activations from the atria to the ventricle are blocked, for example, reducing their probability of causing a capture in the ventricle by, for example, 20%, 50%, 80%, 90%, 95%, 98% or greater or intermediate percentages, for example, as measured over a time period of 1 minute, 10 minutes, 1 hour or smaller or intermediate or greater periods. In some exemplary embodiments of the invention, conduction is intentionally enhanced, for example, by 20%, 50%, 100%, 400%, 10000% or smaller, intermediate or greater percentages. Enhancing may be useful for patients with transient AV block.

An aspect of some embodiments of the invention relates to stimulating one or more AV node extensions and/or parts thereof. Optionally, the stimulating is from within a CS, for example, a CS ostium and/or further inside the CS. Optionally, the stimulation is directional (e.g., is directed to less than 360 degrees (or less than 270, 180, 90, 45 or smaller or intermediate angles in degrees) extending away from the CS, for example, while still having a significant clinical effect (e.g., of reducing probability of activation propagation from an atria to a ventricle by more than 10%). In some embodiments, stimulation is from within the atria, for example, using a screw-in electrode or other attachment method to attach a stimulation electrode to tissue overlying such an extension. Optionally, the AV node is first identified and then one or more electrodes attached at one or more extensions and/or extension portions thereof.

In some exemplary embodiments of the invention, tissue which is not part of the AV node and/or extensions thereof is not stimulated by the signal targeting the AV node and/or extensions thereof in a way that causes a more than 5% change in cardiac output or heart rate.

An aspect of some embodiments of the present invention relates to at least one electrode combined with at least one additional element, placed within the coronary sinus and configured to apply an electric field to affect signal propagation between the atria and ventricles. In some exemplary embodiments of the invention, the additional element is selected from a list consisting of at least one pulse generator, at least one power source (e.g., a battery and/or power circuitry), and at least one processor and/or other control circuitry. In some embodiments the stimulation electrodes are positioned in the proximal part of the CS in locations that are in close vicinity to signal conduction pathways. In some embodiments electrodes are positioned in locations with high probability to affect signal conduction between atria and ventricles. In some embodiments electrodes are ring electrodes or point electrodes or a combination of both ring and point electrodes. In some embodiments electrodes are combined with an anchoring element, configured to anchor the electrode within the coronary sinus. In some embodiments the anchoring element is a hook. Alternatively, anchoring element is an adjustable cylindrical mesh or grid. In some embodiments, electrodes are attached to or buried within the CS wall. In some embodiments, electrodes are located in proximity to the CS wall.

An aspect of some embodiments of the present invention relates to mapping the effect of the electric field applied by each electrode or electrode set placed within the coronary sinus or proximal to the coronary sinus, on signal propagation between the atria and ventricles. In some embodiments mapping comprises applying an electric field through at least one electrode, sensing at least one heart activity parameter by at least one heart activity sensor, and analyzing and determining whether the applied electric field resulted with a desired effect. If the desired effect was not reached, then another electric field is applied through other electrode or set of electrodes, followed by analysis and determination, until the desired effect is reached. In some embodiments, mapping initiates with an electrode or an electrode set located in proximity to atria-ventricles signal conduction pathways. Alternatively, mapping initiates with an electrode or an electrode set, located at the proximal part of the CS. In some embodiments mapping initiates with an electrode or an electrode set, located near the CS ostium. In some embodiments, mapping initiates with an electrode or an electrode set placed in a location that has high probability in affecting signal conduction pathways between the atria and ventricles.

An aspect of some embodiments of the present invention relates to an implantable system, optionally in the form of a unitary device or a two part device (e.g., casing and leads), placed at least partly within the CS, configured to apply an electric field to affect signal conduction between atria and ventricles. The implantable system comprises at least one electrode, at least one heart activity sensor, and at least one processor, and at least one pulse generator. In some embodiments, heart activity sensor is selected from the group of atrial activity sensor, and ventricle activity sensor. In some embodiments the implanted system is a leadless system. In some embodiments the implantable system is configured for electrode mapping and electric field application. In some embodiments, electric field is applied according to pre-defined application protocols selected by the processor. In some embodiments application protocols include protocols for treatment of atrial fibrillation symptoms. In some embodiments application protocols include protocols combining application of both sub-threshold and supra-threshold electric fields. In some embodiments, the processor is receiving signals from at least one heart activity sensor, analyzing the received signal, selecting an electric field application protocol and signals the pulse generator to initiate pulse generation. In some embodiments, the electrodes are combined with an anchoring element configured to anchor the system within or partially within the coronary sinus. In some embodiments the anchoring element has a closed and open conformation states. In some embodiments the anchoring element is an adjustable cylindrical mesh or grid, optionally sized for implantation in an adult or smaller sized human.

The present disclosure provides, according to some embodiments, methods and/or systems for applying an electric field to a heart, for example to provide atrial fibrillation therapy to a subject in need thereof. The present disclosure, in some embodiments thereof, is based in part on the effect of delivering sub-threshold electrical bursts to the atrioventricular (AV) node and/or associated tissue of a subject. Without wishing to be necessarily bound by any theory or mechanism, delivering sub-threshold electrical bursts to the atrioventricular (AV) node of a subject's heart prolongs the effective refractory period (ERP) of myocytes in the AV node, thus slowing the ventricular contraction rate. It is noted that various effects on the conductivity of tissue in the AV node may be achieved, for example as described herein, for example, using different pulse parameters.

In some embodiments described herein the terms “sub-threshold electrical bursts”, “sub-threshold bursts” and “the bursts” are used interchangeably and refer to electrical bursts having a lower current, voltage, frequency, duration and/or timing or other properties than required to induce action potential of myocardial cells, specifically of myocardial cells at the AV node. According to some embodiments, sub-threshold electrical bursts are bursts having either a single, continuous or multiple discrete stimuli. Each possibility represents a separate embodiment of the present invention. In some exemplary embodiments of the invention, the burst are also selected to be sub-threshold to nearby tissue which may be inadvertently electrified by bursts meant to target the AV node (or other pathways) and/or associated tissue. In some exemplary embodiments of the invention, bursts are selected to be supra-threshold, for example, as described herein.

According to some embodiments, delivering sub-threshold electrical bursts to the atrioventricular (AV) node of a subject undergoing atrial fibrillation (AF) or other arrhythmia (AF being used typically as an example in the specification but not necessarily excluding other atrially mediated arrhythmia) may serve to maintain a desired parameter, for example value in range, value matching cardiac demand and/or reduce symptoms associated with atrial fibrillation, such as, but not limited to, tachycardia. As generally described in the art, the AV node is a compact area which may be difficult to access in a precise manner (Willems et al., J Am Coll Cardiol. 1997; 29(2):408-415).

Potentially advantageously, the present disclosure provides, according to some embodiments thereof, a system having a plurality of electrodes configured to be situated at or near the subject's atrioventricular (AV) node. According to some embodiments, delivering sub-threshold electrical bursts to the AV node using at least one of the plurality of electrodes, or a combination thereof, may provide the desired effect of attaining a normal ventricular contraction rate. According to some embodiments, the disclosed system is configured to determine which of the electrodes or combination thereof is correctly positioned such that it is able to deliver sub-threshold electric bursts to the subject's AV node resulting in a ventricular rate within a pre-determined range.

According to some embodiments, the disclosed system comprising a plurality of electrodes obviates the need of precisely positioning a single electrode at the AV node in order to achieve the desired ventricular rate. According to some embodiments, the disclosed system is configured to determine which electrodes (or combinations thereof) are correctly positioned to deliver the sub-threshold bursts to the AV node, thus achieving the desired ventricular rate.

Without wishing to be necessarily bound by any theory or mechanism, if the electrodes which deliver sub-threshold electric bursts to the subject's AV node are not correctly positioned, the bursts may not have a desired effect on the AV node and thereby fail to induce a desired ventricular rate/effect and/or may not have a desired temporal effect on the AV node which effect is able to prevent atrial fibrillation signals from arriving at the AV node. According to some embodiments, the disclosed system is further configured to determine which of the electrodes or combination thereof is able to deliver sub-threshold electric bursts to the subject's AV node and prevent atrial fibrillation signals from arriving at the AV node in between bursts. Atrial fibrillation signals which arrive at the AV node may disrupt normal heart rate and induce various side-effects associated with heart arrhythmia.

According to some embodiments, the disclosed system comprises a tubular electrode anchor, such as a stent configured to be inserted into the coronary sinus, wherein the stent is configured to function as at least one electrode and/or comprises at least one electrode, preferably comprising a plurality of electrodes. Each possibility represents a separate embodiment of the present invention.

According to some embodiments, the electrodes in the stent are configured to deliver sub-threshold electric bursts to the subject's atrioventricular (AV) node. Potentially advantageously, using a stent configured to function as at least one electrode and/or comprising at least one electrode enables affixing the at least one electrode firmly within the coronary sinus. Without wishing to be necessarily bound by theory or mechanism, the proximity of the coronary sinus to the AV node enables to precisely provide sub-threshold electrical bursts to the AV node using electrodes encompassed in a stent positioned in the coronary sinus. According to some embodiments, the stent may be easily inserted into and affixed within the coronary sinus. According to some embodiments, the disclosed system is an essentially leadless system comprising a stent configured to be at least partially inserted into the coronary sinus of the subject's heart, wherein the stent comprises and/or is attached to and/or is integrally formed with at least one element of the disclosed system, possibly functionally attached to all elements of the disclosed system. Each possibility represents a separate embodiment of the present invention.

According to one aspect, the present disclosure provides an implantable electrical stimulation system for providing atrial fibrillation therapy to a subject, the system comprising:

at least one electrical-pulse generator configured to generate sub-threshold electric bursts;

a plurality of electrodes functionally connected to said at least one electrical pulse generator, wherein said plurality of electrodes are configured to be situated at or near the subject's atrioventricular (AV) node and wherein said electrodes are configured to deliver said sub-threshold electric bursts to the subject's AV node;
at least one heart-activity sensor; and
a processor configured to:
   receive input from said at least one heart-activity sensor;
determine whether said subject is undergoing atrial fibrillation based on at least part of said input;
measure the ventricular rate of said subject using at least part of said input;
select a subset of one or more electrodes of said plurality of electrodes which are correctly positioned to deliver sub-threshold electric bursts to the subject's AV node such that said bursts induce a ventricular rate within a pre-determined range; and
actuate delivery of sub-threshold electric bursts through said subset of one or more electrodes to the subject's AV node if said subject is undergoing atrial fibrillation and has a ventricular rate above the pre-determined range. Each possibility represents a separate embodiment of the present invention.

According to some embodiments, the implantable electrical stimulation system is an essentially leadless system. In some exemplary embodiments of the invention, a leadless system refers to a system essentially devoid of wires which stretch between elements of the system (e.g., outside a casing) such that wires are stretched within or around the heart. According to some embodiments, a leadless system is a system in which each element is attached to or integrally formed with at least one other system element. According to some embodiment, the entire system is leadless and configured to be at least partially inserted into the coronary sinus of the subject's heart.

According to some embodiments, the implantable electrical stimulation system comprises a stent configured to be at least partially inserted into the coronary sinus of the subject's heart. According to some embodiments, all elements of the system are attached to and/or integrally formed with the stent configured to be at least partially inserted into the coronary sinus of the subject's heart. Each possibility represents a separate embodiment of the present invention.

According to some embodiments, the stent configured to be at least partially inserted into the coronary sinus of the subject's heart comprises or is attached to or is integrally formed with at least one element selected from the group consisting of: at least one heart-activity sensor, at least one electric pulse generator, at least one electrode, a plurality of electrodes, an energy source, a processor and a combination thereof. Each possibility represents a separate embodiment of the present invention.

According to some embodiments, the stent comprises at least one electrode. According to some embodiments, the stent comprises the plurality of electrodes. According to some embodiments, at least part of the system is attached to or integrally formed with a stent configured to be at least partially inserted into the coronary sinus of the subject's heart.

According to some embodiments, the implantable electrical stimulation system comprises an energy source. An energy source may be, but not limited to, a battery. According to some embodiments, the energy source is attached to or integrally formed with at least one element of the system. According to some embodiments, the energy source is functionally connected to at least one element of the system, such as, but not limited to, the processor, the at least one sensor and the at least one electric pulse generator. Each possibility represents a separate embodiment of the present invention. According to some embodiments, the energy source is connected to at least one element of the system, such that the system does not require an additional external energy source. According to some embodiments, the energy source is connected to at least one element of the system in a leadless manner, such that the connection does not require stretching electrical wires or leads within or around the heart.

According to some embodiments, the sub-threshold electric bursts are selected from the group consisting of: a continuous burst, a single burst, a multiple-discrete burst and a combination thereof. Each possibility represents a separate embodiment of the present invention.

According to some embodiments, the sub-threshold electric bursts have a current of between about 0.1 mA to about 5 mA. According to some embodiments, the sub-threshold electric bursts are induced in intervals of between about 0.5 s to about 5 sec.

According to some embodiments, the plurality of electrodes is incorporated into a unitary surface. According to some embodiments, the plurality of electrodes is incorporated in a stent configured to be inserted into the coronary sinus. According to some embodiments, each electrode of said plurality of electrodes is configured to provide the same or different sub-threshold electric burst. Each possibility represents a separate embodiment of the present invention. According to some embodiments, at least part of the plurality of electrodes is configured to be coupled with the surface of the AV node. In some exemplary embodiments of the invention, the coupling with comprises parts that are attached to or at least partially touching. According to some embodiments, the plurality of electrodes is configured to be situated within or adjacent to the coronary sinus.

According to some embodiments, the heart-activity sensor is selected from the group consisting of: atrial sensor configured to sense atrial activity, ventricular sensor configured to sense ventricular activity and a combination thereof. Each possibility represents a separate embodiment of the present invention.

According to some embodiments, the processor is functionally connected to the at least one heart-activity sensor. According to some embodiments, the processor is attached to or integrally formed with the at least one heart-activity sensor.

According to some embodiments, ventricular activity is selected from the group consisting of: ventricular contraction, ventricular rate, ventricular depolarization, ventricular repolarization and a combination thereof. Each possibility represents a separate embodiment of the present invention.

According to some embodiments, atrial activity is selected from the group consisting of: atrial contraction, atrial rate, atrial depolarization, atrial repolarization and a combination thereof. Each possibility represents a separate embodiment of the present invention.

According to some embodiments, delivering sub-threshold electric bursts to the AV node is delivering such that the effective refractory period (ERP) of myocytes in the AV node is prolonged. According to some embodiments, determining whether said subject is undergoing atrial fibrillation is effected using an Automatic Mode Switching algorithm.

According to some embodiments, selecting the subset of one or more electrodes comprises:

inducing delivery of sub-threshold electric bursts to the subject's AV node through at least one of said plurality of electrodes;

determining whether the subject's ventricular rate is within said pre-determined range following the induction; and

if the ventricular rate is not within said pre-determined range following the induction, sequentially repeating said inducing and determining, each induction using a different electrode or combination thereof until the ventricular rate of said subject is within said pre-determined range. According to some embodiments, the pre-determined range is about 100 Beats Per Minute. According to some embodiments, the ventricular rate within a pre-determined range is a ventricular rate which is maintained for at least 1 to 5 minutes.

According to some embodiments, the present invention provides the disclosed system for use in treating atrial fibrillation in a subject.

According to another aspect, the present disclosure provides, in some embodiments thereof, an implantable electrical stimulation system for providing atrial fibrillation therapy to a subject, wherein the system is configured to be situated at least partly within the coronary sinus of the subject's heart, the system comprising:

at least one heart-activity sensor;

at least one electrode functionally connected to said at least one electrical pulse generator and configured to deliver said sub-threshold electric bursts to the subject's atrioventricular (AV) node; and

a processor configured to:

receive input from said at least one heart-activity sensor;
measure the ventricular rate of said subject using at least part of said input; determine whether said subject is undergoing atrial fibrillation based on at least part of said input;
induce delivery of sub-threshold electric bursts to the subject's AV node through said at least one electrode if said subject is undergoing atrial fibrillation and has a ventricular rate above a pre-determined range.

According to some embodiments, the system is in the form of a stent configured to be situated at least partly within the coronary sinus of said subject. According to other embodiments, the system comprises a stent configured to be situated at least partly within the coronary sinus of said subject.

According to some embodiments, the stent comprises or is attached to or is integrally formed with at least one of: said at least one electrical-pulse generator, said at least one heart-activity sensor, said at least one electrode, said processor and a combination thereof. Each possibility represents a separate embodiment of the present invention.

According to some embodiments, the present invention provides an implantable electrical stimulation system for providing atrial fibrillation therapy to a subject, the system comprising:

at least one electrical-pulse generator configured to generate sub-threshold electric bursts;
at least one heart-activity sensor;
a stent configured to be inserted into the coronary sinus, wherein said stent comprises at least one electrode, wherein said at least one electrode is functionally connected to said at least one electrical pulse generator and configured to deliver said sub-threshold electric bursts to the subject's atrioventricular (AV) node; and
a processor configured to:
receive input from said at least one heart-activity sensor;
measure the ventricular rate of said subject using at least part of said input;
determine whether said subject is undergoing atrial fibrillation based on at least part of said input;
induce delivery of sub-threshold electric bursts to the subject's AV node through said at least one electrode if said subject is undergoing atrial fibrillation and has a ventricular rate above a pre-determined range.

According to some embodiments, the present invention provides the disclosed system for use in treating atrial fibrillation in a subject.

According to some embodiments, the present invention provides an implantable electrical stimulation system for providing atrial fibrillation therapy to a subject, wherein at least part of the system is comprised in a stent configured to be at least partly inserted into the coronary sinus of the subject's heart, the system comprising:

at least one electrical-pulse generator configured to generate sub-threshold electric bursts;
at least one heart-activity sensor;
at least one electrode, wherein said at least one electrode is functionally connected to said at least one electrical pulse generator and configured to deliver said sub-threshold electric bursts to the subject's atrioventricular (AV) node; and
a processor configured to:
receive input from said at least one heart-activity sensor;
measure the ventricular rate of said subject using at least part of said input; determine whether said subject is undergoing atrial fibrillation based on at least part of said input;
induce delivery of sub-threshold electric bursts to the subject's AV node through said at least one electrode if said subject is undergoing atrial fibrillation and has a ventricular rate above a pre-determined range.

According to some embodiments, the heart-activity sensor is selected from the group consisting of: atrial sensor configured to sense atrial activity, ventricular sensor configured to sense ventricular activity and a combination thereof. Each possibility represents a separate embodiment of the present invention.

According to some embodiments, the entire system is comprised in the stent configured to be at least partially inserted into the coronary sinus of said subject's heart.

According to some embodiments, the present disclosure provides an implantable electrical stimulation system for providing atrial fibrillation therapy to a subject, the system comprising:

at least one electrical-pulse generator configured to generate sub-threshold electric bursts;
at least one heart-activity sensor;
at least one electrode functionally connected to said at least one electrical pulse generator and configured to deliver said sub-threshold electric bursts to the subject's atrioventricular (AV) node;
a processor configured to:
receive input from said at least one heart-activity sensor;
measure the ventricular rate of said subject using at least part of said input; determine whether said subject is undergoing atrial fibrillation based on at least part of said input;
induce delivery of sub-threshold electric bursts to the subject's AV node through said at least one electrode if said subject is undergoing atrial fibrillation and has a ventricular rate above a pre-determined range; and
a stent configured to be inserted into the coronary sinus of the subject's heart, wherein said stent comprises or is attached to or is integrally formed with at least one of: said at least one electrical-pulse generator, said at least one heart-activity sensor, said at least one electrode, said processor and a combination thereof. Each possibility represents a separate embodiment of the present invention.

According to some embodiments, the at least one electrode is a plurality of electrodes. According to some embodiments, the processor is further configured to determine which subset of the plurality of electrodes is able to deliver sub-threshold electric bursts to the subject's AV node such that said bursts induce a ventricular rate within a pre-determined range; and actuate delivery of sub-threshold electric bursts through said subset.

According to some embodiments, the electrodes in the subset are correctly positioned to deliver sub-threshold electric bursts to the subject's AV node such that said bursts induce a ventricular rate within the pre-determined range. According to some embodiments, determining the subset comprises:

inducing delivery of sub-threshold electric bursts to the subject's AV node through at least one of said electrodes;

determining whether the subject's ventricular rate is within said pre-determined range following the induction; and

if the ventricular rate is not within said pre-determined range following the induction, sequentially repeating said inducing and determining, each induction using a different electrode or combination thereof until the ventricular rate of said subject is within said pre-determined range.

According to another aspect, the present invention, in some embodiments thereof, provides a method of treating atrial fibrillation in a subject, the method comprising:

selecting a correctly positioned subset of one or more electrodes of a plurality of electrodes positioned at or near the subject's AV node, wherein said subset is able to deliver sub-threshold electric bursts to the AV node such that said bursts induce a ventricular rate within a pre-determined range; and
inducing delivery of sub-threshold electric bursts to the AV node through said subset of one or more electrodes.

The method of some embodiments of the invention may be facilitated using the system of the invention. According to some embodiments, the method of the invention further comprises positioning at least part of the disclosed system in the subject's heart, preferably at least partially within the coronary sinus of the subject's heart. Each possibility represents a separate embodiment of the present invention.

According to some embodiments, the method further comprises positioning said plurality of electrodes at or near the subject's atrioventricular (AV) node, wherein the electrodes are configured to deliver sub-threshold electric bursts to the subject's AV node.

According to some embodiments, the method further comprises positioning said plurality of electrodes at or near the subject's parasympathetic plexi of the heart, wherein the electrodes are configured to deliver sub-threshold electric bursts to at least part of the parasympathetic plexi of the subject's heart.

According to some embodiments, positioning comprises positioning at least part of said plurality of electrodes near and/or within the coronary sinus. Each possibility represents a separate embodiment of the present invention. According to some embodiments, the plurality of electrodes is comprised in a stent. According to some embodiments, the method further comprises positioning the stent within the coronary sinus.

According to some embodiments, positioning comprises positioning each electrode of said plurality of electrodes at a different position at or near the AV node. According to some embodiments, positioning comprises positioning each electrode of said plurality of electrodes at a different position at or near the parasympathetic plexi of the subject's heart.

According to some embodiments, selecting the subset of one or more electrodes comprises:

inducing delivery of sub-threshold electric bursts to the subject's AV node through at least one of said plurality of electrodes;
determining whether the subject's ventricular rate is within said pre-determined range following the induction; and
if the ventricular rate is not within said pre-determined range following the induction, sequentially repeating said inducing and determining, each induction using a different electrode or combination thereof until the ventricular rate of said subject is within said pre-determined range; wherein electrodes able to deliver sub-threshold electric bursts to the subject's AV node are correctly positioned electrodes.

According to some embodiments the method further comprises determining whether the subject is undergoing atrial fibrillation. According to some embodiments the delivery of the sub-threshold electric bursts according to the disclosed method is induced only if the subject is undergoing atrial fibrillation and has a ventricular rate above the pre-determined range.

There is provided, in accordance with some exemplary embodiments, an apparatus for cardiac electrification, comprising: (a) at least one electrode sized and shaped for placement within an adult coronary sinus of a heart; (b) a signal generator electrically coupled to the electrode and configured to electrify the electrode so that the electrode applies an electric field to an atrial-ventricular conduction pathway or associated tissue and which field is configured to modify conduction of an atrial activation through the pathway to activate a ventricle.

According to some embodiments, the at least one electrode is mounted on a structure sized and shaped for anchoring in the cardiac sinus.

According to some embodiments, the structure is sized and pre-deformed to self-expand to anchor in the coronary sinus.

According to some embodiments, the apparatus comprises an electrode array sized and shaped to conform to an inner surface of the coronary sinus, at multiple axial and circumferential locations thereof, simultaneously.

According to some embodiments, the array comprises an annular array including the at least one electrode, the array having a diameter which corresponds to a diameter of a proximal side of an adult coronary sinus.

According to some embodiments, the annular array includes at least two spatially separate electrodes at a same axial location and different circumferential locations.

According to some embodiments, the array comprises a ring electrode.

According to some embodiments, the at least one electrode comprises a plurality of separately electrifiable electrodes sized and shaped for simultaneous insertion into the cardiac sinus.

According to some embodiments, the at least one electrode comprises at least four electrodes sized and shaped for simultaneous insertion into the cardiac sinus. According to some embodiments, at least one of the at least one electrode is sized and shaped for positioning within 5 mm of an ostium of the coronary sinus. According to some embodiments, at least one of the at least one electrode is sized and shaped for positioning wholly within 7 mm of an ostium of the coronary sinus. According to some embodiments, the apparatus comprises at least one electrode configured for attachment to cardiac muscle in or on an atria or ventricle.

According to some embodiments, the pathway comprises an AV node.

According to some embodiments, the electrode and electrification are configured to electrify tissue which acts as input to an AV node.

According to some embodiments, the electrode and electrification are configured to modify conduction, at least mostly, by the action of the field on muscle fibers.

According to some embodiments, the modify conduction comprises blocking conduction of at least 20% of activations passing through the pathway, from reaching the ventricle with an amplitude and timing sufficient to activate the ventricle.

According to some embodiments, the blocking comprises blocking while the field is applied.

According to some embodiments, the electrode and electrification are configured to avoid direct modification of conduction or activation in non-target tissue which is not of the pathway and the associated tissue, while having the modifying effect on the pathway tissue, other than a volume of non-target tissue which is at most 1 cubic cm in volume.

According to some embodiments, the field is sub-threshold to the pathway and associated tissue in that it does not generate a new propagating action potential, which can propagate further than 5 mm, therein.

According to some embodiments, the field is sub-threshold to the heart in that it does not generate a new propagating action potential, which can propagate further than 5 mm, therein.

According to some embodiments, the field is sub-threshold to cardiac muscle tissue in that it does not generate a new propagating action potential, which can propagate further than 5 mm, therein.

According to some embodiments, the electrifying comprises electrifying the at least one electrode with a field that is 0.1-5 mA and/or 0.05-10 or 15 volts. According to some embodiments, the apparatus comprises circuitry which controls the signal generator.

According to some embodiments, the apparatus comprises at least one physiological sensor.

According to some embodiments, the sensor generates a demand indication and wherein the circuitry modifies the electrifying in response to the indicated demand.

According to some embodiments, the sensor generates an indication of existing or incipit atrial arrhythmia and wherein the circuitry modifies the electrifying in response to the indication.

According to some embodiments, the sensor generates an indication of ventricular timing and wherein the circuitry modifies the electrifying in response to the indication.

According to some embodiments, the sensor generates an indication of ventricular rate and wherein the circuitry modifies the electrifying in response to the indication.

According to some embodiments, at least one of the at least one electrodes is used as the sensor.

According to some embodiments, the circuitry controls the electrification to create temporal windows in the activity of the pathway within which an activation for the atria is more likely to reach a ventricle than outside the window.

According to some embodiments, the circuitry is configured to prevent or reduce symptoms of one or more of atrial fibrillation, atrial flutter, atrial tachycardia and/or any supra-ventricular tachycardia by selectively blocking electrical activation of a ventricle from an atria or AV node.

There is provided, in accordance with some exemplary embodiments, an apparatus for cardiac electrification, comprising: (a) at least one electrode; (b) a pulse generator configured to electrify the at least one electrode; (c) control circuitry configured to control the electrification by the pulse generator; (d) a power source; and (e) a casing encompassing at least one of b-d, and sized for insertion into and anchoring in a coronary sinus of an adult human heart.

According to some embodiments, the casing includes an anchoring component extending distally away from the casing.

According to some embodiments, the anchoring component radially self expands to anchor in the coronary sinus.

According to some embodiments, the at least one electrode is mounted on the casing.

According to some embodiments, the apparatus further comprises sensing circuitry which generates an indication of a physiological parameter related to the heart, the sensing circuitry communicating the indication to the control circuitry.

There is provided, in accordance with some exemplary embodiments, a method for modifying electrical activity in the heart, comprising: (a) applying an electric field to an AV node and/or associated tissue using an electrode located within a coronary sinus; and (b) modifying conduction of atrial activation through the AV node to a ventricle by the applying.

According to some embodiments, the applying comprises applying an electric field which is sub-threshold to the AV node and associated tissue in that it does not generate a new propagating action potential, which can propagate further than 5 mm, therein.

According to some embodiments, the applying comprises directly affecting muscle fibers in the AV node and/or associated tissue to achieve at least most of the modifying.

According to some embodiments, the applying comprises avoiding affecting nervous tissue in a manner which will have an effect of more than 10% on heart rate or stroke volume.

According to some embodiments, the applying comprises avoiding affecting tissue of a volume greater than twice the AV node and/or associated tissue.

According to some embodiments, the applying comprises avoiding affecting tissue of a volume greater than twice the AV node and/or associated tissue.

According to some embodiments, the applying comprises blocking at least 30% of the activations to a degree that they do not activate the ventricle.

According to some embodiments, the applying comprises treating or preventing VT caused by atrial arrhythmia, by the modifying.

According to some embodiments, the applying comprises applying responsive to a state of arrhythmia in an atria.

According to some embodiments, the applying comprises applying responsive to a state of arrhythmia in a ventricle.

According to some embodiments, the applying comprises applying responsive to a heart rate in a ventricle.

According to some embodiments, the applying comprises adjusting the applying to achieve a heart rate in a ventricle within a range.

According to some embodiments, the applying comprises generating a temporal window within which activation passage from the atria to the ventricle is better than outside the window.

According to some embodiments, the applying comprises repeating the applying at least 10 times a minute during atrial arrhythmia.

According to some embodiments, comprising modifying at least one of an electrode used for the applying and at least one parameter used for the applying, responsive to an efficacy thereof.

According to some embodiments, comprising modifying at least one of an electrode used for the applying and at least one parameter used for the applying, responsive to a side-effect thereof.

There is provided, in accordance with some exemplary embodiments, a method of electrode selection, comprising: (a) providing a plurality of electrodes anchored to cardiac tissue; (b) electrifying an AV node or associated tissue using at least one electrode of the plurality of electrodes; (c) determining an effect of the electrification; and (d) repeating (b)-(c) using a different at least one electrode of the plurality, responsive to the determined effect.

According to some embodiments, providing comprises anchoring the plurality of electrodes within a cardiac sinus.

According to some embodiments, (b)-(d) compensate for an accuracy of anchoring of the plurality of electrodes.

According to some embodiments, the method comprises selecting at least one of the plurality of electrodes responsive to (b)-(d).

According to some embodiments, selecting comprises selecting according to efficacy.

According to some embodiments, efficacy comprises a degree of blocking of activation from an atria to a ventricle through the AV node.

According to some embodiments, selecting comprises selecting according to a side effect of using the selected electrode.

According to some embodiments, electrifying comprises electrifying with a sub-threshold electric field.

According to some embodiments, the method comprises repeating (b) and (c) for a same electrode with different pulse parameters.

According to some embodiments, the at least one electrode and the different at least one electrode are spaced apart less than 4 mm.

There is provided, in accordance with some exemplary embodiments, a method of cardiac treatment, comprising: (a) providing a patient with a cardiac diagnosis; (b) permanently implanting at least one cardiac electrode and at least part of a device casing within a cardiac sinus of the patient; and (c) treating the patient in response to the diagnosis using electrification of the at least one cardiac electrode by a power source within the device casing.

According to some embodiments, the treating comprises directly affecting an AV node and/or associated tissue using the electrification.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as determining if and when to apply a signal and/or what parameters to use, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1A schematically illustrates signal conduction pathways of the heart;

FIG. 1B schematically illustrates the coronary sinus and the AV node;

FIG. 1C schematically illustrates electrodes within the heart in close proximity to the AV node, according to some embodiments of the invention;

FIG. 1D schematically illustrates prophetic examples of electrocardiogram (ECG) recordings during episodes of atrial flutter, atrial fibrillation and atrial tachycardia and after sub-threshold burst application, according to some embodiments of the invention;

FIG. 2A is a block diagram depicting system components according to some embodiments of the invention;

FIG. 2B is a block diagram depicting system interactions with heart tissue, according to some embodiments of the invention;

FIG. 2C is a flow chart scheme depicting the main stages of electric field application, according to some embodiments of the invention;

FIG. 3A is a flow chart scheme depicting a method of electric field application, according to some embodiments of the invention;

FIG. 3B is a flow chart scheme depicting a method of electric field application combined with methods for mapping and electrode selection, according to some embodiments of the invention;

FIG. 3C is a flow chart scheme depicting the disclosed method for mapping and electrode selection, according to some embodiments of the invention;

FIG. 3D schematically illustrates the disclosed implantable system comprising a plurality of electrodes, according to some embodiments of the invention;

FIG. 3E schematically illustrates an implantable system comprising a stent positioned in the coronary sinus, according to one embodiments of the invention;

FIG. 3F schematically illustrates electrodes within the coronary sinus, according to some embodiments of the invention;

FIG. 3G schematically illustrates a stent comprising electrodes within the coronary sinus, according to some embodiments of the invention;

FIG. 4A schematically illustrates an implantable leadless system comprising a stent configured to be at least partially positioned in the coronary sinus, according to some embodiments of the invention; and

FIG. 4B schematically illustrates an enlargement of part of a stent positioned at the proximal end of the leadless system, according to some embodiments of the invention;

FIG. 5 schematically illustrates a leadless system within the coronary sinus, according to some embodiments of the invention; and

FIGS. 6A-F schematically illustrate embodiments of electrodes combined with supportive elements of the system configured to be placed within the coronary sinus, according to some embodiments of the invention;

### DESCRIPTION OF SPECIFIC EMBODIMENTS OF THE INVENTION

The present invention, in some embodiments thereof, relates to applying an electric field to the heart and, more particularly, but not exclusively, to a method and system for affecting signal conduction pathways between the atria and ventricles, for example, for treating the symptoms of atrial fibrillation.

### Overview

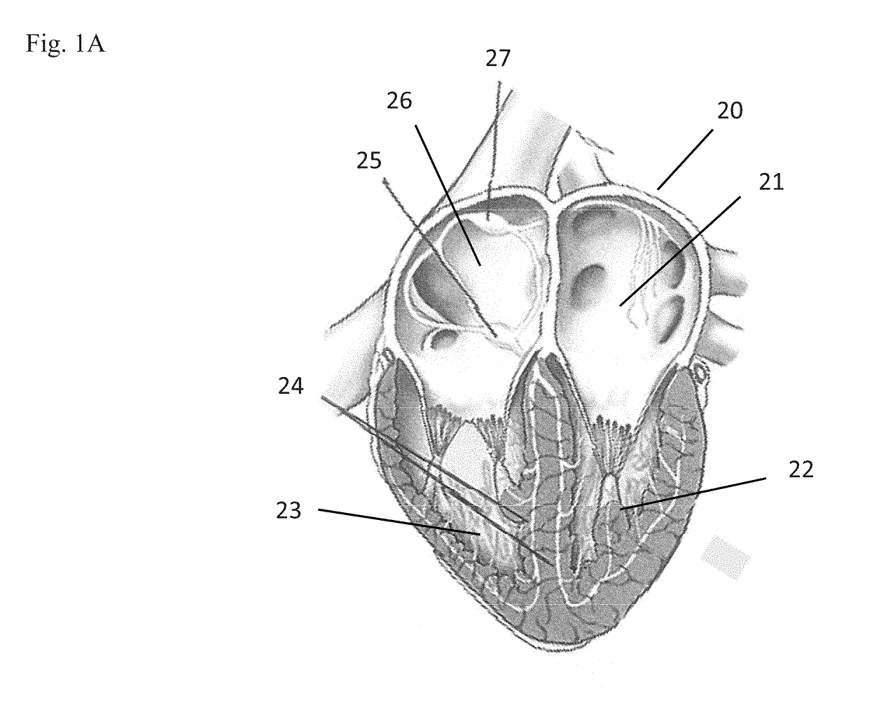

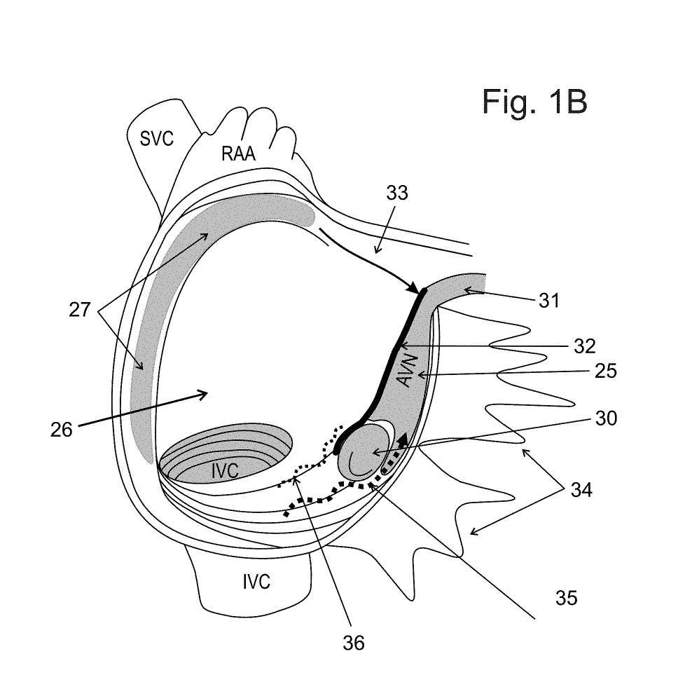

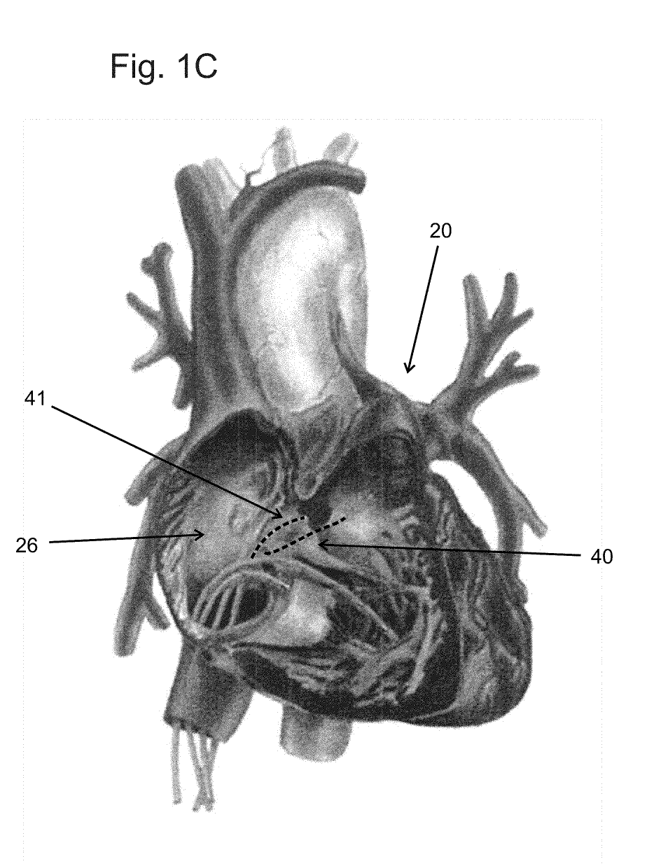

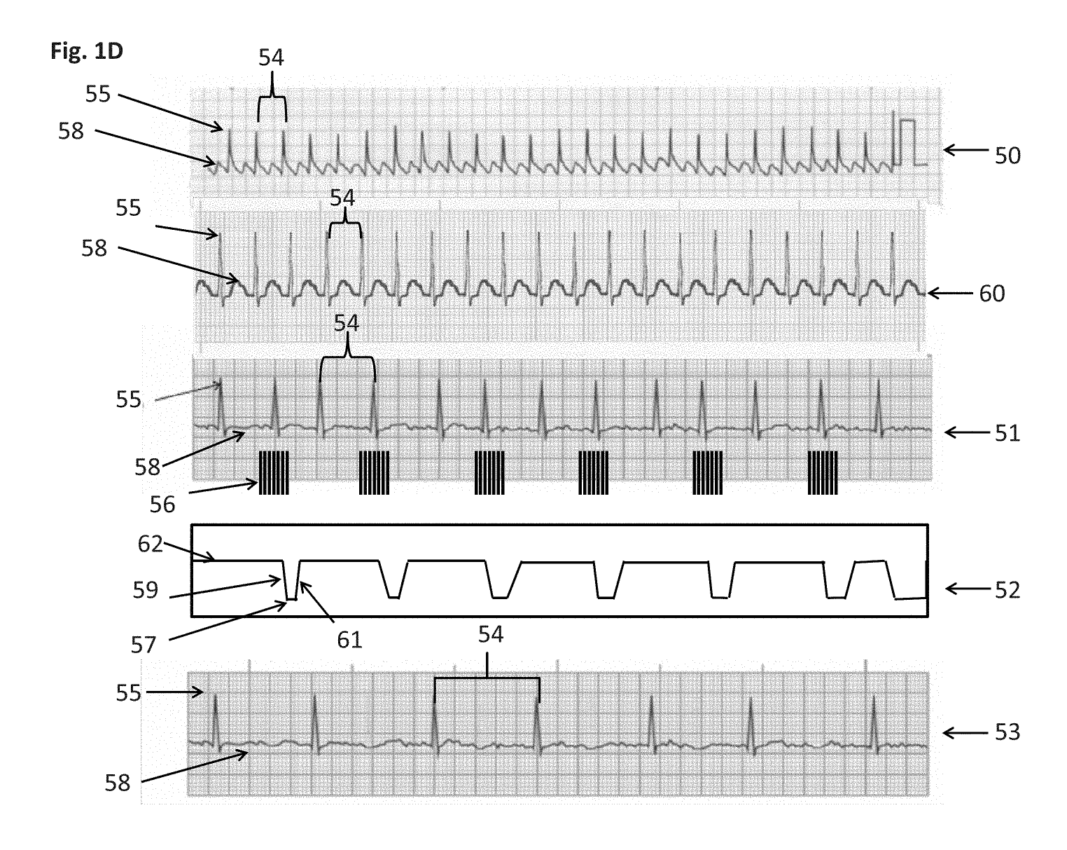

Referring to FIG. 1A which shows a heart; the present invention provides, according to some embodiments, implantable systems which are configured to apply an electric field from within a coronary sinus to affect signal propagation between the atria and ventricles of a heart 20. In some embodiments the applied electric field interferes with the propagation of signals from a left atrium 21 and/or a right atrium 26 to a left 22 and/or a right 23 ventricle, allowing only selective signals to reach the ventricles. In some embodiments, the applied electric field interferes with the propagation of signals from Sinoatrial node (SA node) 27 to AV node 25. In some embodiments, signals arriving to the AV node are delivered to the ventricles through bundle of His and Purkinje Fibers 24. In some embodiments electric field application allows to increase the number of signals arriving to the ventricles. In some embodiments the applied electric field is a sub-threshold electric field, which is below the threshold required to induce an action potential or to cause cell contraction in tissue to which it is applied. In some embodiments, application of a sub-threshold electric field is combined with application of an electric field which is supra-threshold. In some embodiments the sub-threshold electric field or supra-threshold electric field is applied as a continuous burst, a single burst, a multiple-discrete burst or a combination thereof.

The present invention provides, according to some embodiments, implantable systems which are configured to apply an electric field from within a coronary sinus to affect signal propagation between the atria and ventricles of a heart 20. In some embodiments the applied electric field interferes with the propagation of signals from a left atrium 21 and/or a right atrium 26 to a left 22 and/or a right 23 ventricle, allowing only selective signals to reach the ventricles. In some embodiments electric field application allows to increase the number of signals arriving to the ventricles. In some embodiments the applied electric field is a sub-threshold electric field, which is below the threshold required to induce an action potential or to cause cell contraction in tissue to which it is applied. In some embodiments, application of a sub-threshold electric field is combined with application of an electric field which is supra-threshold. In some embodiments the sub-threshold electric field or supra-threshold electric field is applied as a continuous burst, a single burst, a multiple-discrete burst or a combination thereof.

In some embodiments the applied electric field interacts directly with AV node 25, for example with muscle fibers therein. Optionally or alternatively, in some embodiments the applied electric field indirectly affects signal conduction through AV node 25, by affecting muscle tissue at the vicinity of AV node 25. In some embodiments, the applied electric field affects AV node 25 by affecting muscular tissue leading into AV node 25, instead or in addition to neural tissue. The AV node region is located in the lower portion of the right atrium near the right ventricle, at the apex of the triangle of Koch, which is an anatomically triangular region on the septal wall of the right atrium demarcated by a tendon of Todaro 32, a septal leaflet of the tricuspid valve 34, and the orifice of the coronary sinus, also termed a coronary sinus ostium 30.

In some embodiments, the applied electric field directly affects AV node input pathways, for example a left 33 and a right 36 and 35 extensions (e.g., inferior and/or posterior) of AV node 25. The left and right AV node extensions are also termed the “fast” and “slow” signal conduction pathways, respectively.

The present disclose provides, according to some embodiments, implantable systems which are configured to provide atrial fibrillation therapy to a subject by delivering sub-threshold electrical bursts to the AV node of the subject's heart. According to some embodiments, the system is configured to modulate the depolarization (e.g., rate thereof) of the subject's AV node. In some exemplary embodiments of the invention, the modulation is used to control the ventricular contraction (e.g., rate) without needing to ablate the AV node and/or implant a pacemaker. According to some embodiments, the disclosed system obviates the need for precisely positioning an electrode at the AV node as it is able to determine which electrode (or electrode combination) that is positioned in the proximity of the AV node is able to deliver sub-threshold electric bursts to the AV node, such that the resulting ventricular rate is within a pre-determined range. According to some embodiments, directing sub-threshold electric bursts to the AV node of a subject results in slowing down of the ventricular contraction rate due to elongation of the refractory period of the myocytes at the AV node.

In some exemplary embodiments of the invention, the implants (e.g., electrode and anchoring structure and/or relative positioning of electrodes) is elected to match an adult human. In other embodiments, it may be sized for children or adolescents.

According to one aspect, the present disclosure provides an implantable electrical stimulation system, wherein said system is configured to provide atrial fibrillation therapy to a subject, wherein the system comprises:

at least one electrical-pulse generator configured to generate sub-threshold electric bursts;

a plurality of electrodes functionally connected to said electrical pulse generator, wherein said electrodes are configured to be situated at or near the subject's atrioventricular (AV) node and wherein said electrodes are configured to deliver said sub-threshold electric bursts to the subject's AV node;

at least one heart-activity sensor; and a processor configured to:

receive input from said at least one heart-activity sensor;

determine whether said subject is undergoing atrial fibrillation based on at least part of said input;

measure the ventricular rate of said subject using at least part of said input; select a subset of one or more of said plurality of electrodes which are able to deliver sub-threshold electric bursts to the subject's AV node; and

actuate delivery of sub-threshold electric bursts through said subset of one or more electrodes to the subject's AV node if said subject is undergoing atrial fibrillation and has a ventricular rate above a pre-determined range.

According to some embodiments, said pre-determined range is between 70 and 120 BPM, for example, 90-110 BPM (Beats Per Minute). According to some embodiments, said pre-determined range is between about 100-110 BPM. According to some embodiments, said pre-determined range is about 100 BPM.

In some exemplary embodiments of the invention, the range is adaptable to patient needs, for example, increasing with exercise (e.g., as indicated by an optional accelerometer) and/or decreasing (and/or increasing) at sleep and/or at preset times of the day (e.g., using a clock circuit).

In some embodiments, at least one of said plurality of electrodes is configured (e.g., sized, shaped and/or mounted in a position suitable for) to provide an electric field to tissue associated with the AV node. Referring now to FIG. 1C which shows electrodes within the coronary sinus and at the AV node, while some embodiments target the AV node itself by placing an electrode 41 at the AV node, other embodiments target tissue which affects the AV node, such as one or both AV node extensions. In accordance with some embodiments, said at least one electrode 40 is positioned within the coronary sinus body and/or ostium. Optionally, positioning uses an anchoring structure, for example as described herein.

According to some embodiments, the subset of one or more of the plurality of electrodes is able to deliver sub-threshold electric bursts to the subject's AV node such that the bursts induce a ventricular rate within a pre-determined range. According to some embodiments, the selected subset comprises electrodes which are correctly positioned to be able to deliver sub-threshold electric bursts to the subject's AV node. According to some embodiments, the one or more electrodes of the selected subset are correctly positioned electrodes.

In some exemplary embodiments of the invention, a “subject in need thereof” refers to a subject afflicted with or at risk of being afflicted with atrial fibrillation, typically refractory atrial fibrillation. In some embodiments, the subject has other disorders, for example, other atria-related disorders, such as atrial flutter, atrial tachycardia and/or AV node reentrant tachycardia or other supra-ventricular arrhythmia. In some embodiments, the method and/or system are used for treating non-AV abnormal pathways between atria and ventricles and/or symptoms caused thereby.

According to some embodiments, the disclosed system comprises a plurality of electrodes. According to some embodiments, the electrodes are functionally connected to at least one electric pulse generator configured to generate sub-threshold electric bursts and/or supra-threshold pulses and are further configured to transmit the bursts to target tissue.

According to some embodiments, the disclosed system comprises said at least one electric pulse generator configured to generate sub-threshold electric bursts. According to some embodiments, the electric pulse generator is configured to generate only sub-threshold electric bursts. According to some embodiments, the electric pulse generator comprises a current and/or voltage modulator configured to enable the electric pulse generator to produce sub-threshold and/or supra-threshold electric bursts. Each possibility represents a separate embodiment of the present invention. According to certain embodiments, the current and/or voltage modulator is configured to prevent the electric pulse generator from generating an electric pulse which is not sub-threshold. According to certain embodiments, the current and/or voltage modulator is configured to prevent the electric pulse generator from generating an electric pulse which is not sub-threshold when the subject is undergoing atrial fibrillation.

### Exemplary Applied Signals

According to some embodiments, the electric field applied by at least one electrode is an electric burst. In some embodiments the electric field is applied as a plurality of electric bursts.

In some exemplary embodiments of the invention, sub-threshold electric bursts refer to electrical bursts having a lower current than required to induce action potential of myocardial cells, specifically of myocardial cells at the AV node (and/or other one or more other parameters whose value is not within a window of excitation) and/or of other tissue which may receive an electric field using methods and/or apparatus as described herein.

According to some embodiments, a sub-threshold electric burst refers to a single sub-threshold electric burst. According to some embodiments, a sub-threshold electric burst refers to a continuous sub-threshold electric burst. According to some embodiments, a sub-threshold electric burst refers to an electric burst comprising a plurality of sequential discrete sub-threshold electric bursts (also referred to herein as “a multiple-discreet burst”). According to some embodiments, a sub-threshold electric burst refers to an electric burst selected from the group consisting of: a single burst, a continuous burst and a multiple-discreet electric burst. According to some embodiments, a continuous sub-threshold electric burst refers to a burst lasting about 1 ms-100 ms or about 100 ms to about 5 sec. According to some embodiments, a continuous sub-threshold electric burst refers to a burst lasting about 100-500 ms, 100 ms-1 s or 500 ms-4 sec. Each possibility represents a separate embodiment of the present invention. In some exemplary embodiments of the invention, the burst application is synchronized (e.g., using an electrical activity sensor and synchronizing circuitry) to the cardiac cycle and/or electrical activation in the target tissue and/or nearby non-target tissue. In some exemplary embodiments of the invention, a multi-discrete burst is formed of between 2 and 2000 sub-bursts (e.g., discrete waveforms). Optionally, between 3 and 100 sub-bursts. Optionally, a delay is provided between consecutive bursts, for example, between 1 and 10000 ms, for example, between 50 and 300 ms. Optionally, a delay is provided between sub-bursts, for example, between 0.5 and 100 ms, for example, between 1 and 50 ms. According to some embodiments, bursts can be synchronized (e.g., with a delay) with ventricle contraction (e.g., based on a QRS or an equivalent ventricular activation signal), or applied according to a pre-determined application protocol and/or be unsynchronized. According to some embodiments, bursts may be separated by inter-stimulus intervals or fused to form a continuous burst.

According to some embodiments, a plurality of sequential discrete sub-threshold electric bursts refers to about between 2 and 1000 discrete bursts applied in sequence (e.g., as a single application. Optionally, a burst is stopped periodically, for example, to match a cardiac cycle. A burst meaning a combination of electrical stimuli provided in a pre-programmed and/or adjustable and/or adapting (e.g., responsive to sensor input, possibly using a decision table or other logic) manner with specific stimulus amplitude, width and inter-stimuli interval. According to some embodiments, the plurality of sequential discrete sub-threshold electric bursts within each multiple-discreet burst refers to bursts fired within about 10 to 500 ms. According to some embodiments, a plurality of sequential discrete sub-threshold electric bursts within each multiple-discreet burst refers to about 2-1000 discrete bursts fired within about 1-1000 ms. According to some embodiments, the duration of each discrete sub-threshold electric burst (e.g., a sub-burst) within the plurality of sequential bursts is between about 1 to 1000 ms (ms=milli-seconds). Optionally, a sub-burst comprises a DC signal. Optionally or alternatively, the sub-burst has a more complex waveform, for example, AC.

According to some embodiments, the therapy cycle of the sub-threshold electric bursts is a single, continuous or multiple-discreet sub-threshold electric burst once every about 0.5-5 sec, optionally timed to match existing and/or desired electrical activity in a part of the heart. Each possibility represents a separate embodiment of the present invention. According to some embodiments, the electric pulse generator is configured to provide a sub-threshold electric burst once every 0.5-10 sec. According to some embodiments, the frequency of the therapy cycle increases with an increase in ventricular rate, for example, when the ventricular rate increases over 100 bpm. According to some embodiments, the system's processor is configured to increase the frequency of sub-threshold electric bursts when ventricular rate increases above the pre-determined range and/or decrease the frequency when the ventricular rate decreases up until the pre-determined range. Each possibility represents a separate embodiment of the present invention.

In some exemplary embodiments of the invention, arrhythmia is detected based on a fast ventricular rate. Then, a sub-threshold stimuli is applied, for example, within the CS sinus and/or otherwise to the AV node and/or AV node extensions, optionally starting with a low amplitude and increasing gradually until a reduction in ventricular rate is detected. Optionally, the amplitude starts at 0.1V and optionally increases linearly (e.g., in steps of 0.1V) or non-linearly). Optionally, a last working signal is used as a starting point for a next application.

In one example, bursts start at a lowest programmed amplitude (or other parameter to be varied), increasing in amplitude until either the ventricular rate is reduced below 110 bpm (or any other predetermined target), or the ventricular rate is increased to over 160 bpm (or any predetermined target). Optionally, the burst parameters (e.g., if parameters other than or in addition to amplitude are changed), are optionally stored and used in the next episode of high ventricular rate. Optionally or alternatively, parameters, parameter ranges, thresholds and/or which parameters to vary and/or in what order and/or other search parameters (e.g., which electrode(s) to try) are provided using an external programmer.

According to some embodiments, the current of the sub-threshold electric bursts is between about 0.1 mA to about 5 mA or 10 mA, for example, when measured as a maximum and/or as an average (e.g., for a square wave pulse). According to some embodiments, the current of the sub-threshold electric bursts is between about 0.1 mA to about 4 mA, possibly between about 0.1 mA to about 3 mA. Each possibility represents a separate embodiment of the present invention. According to some embodiments, the current of each discreet burst within the plurality of sub-threshold electric bursts is between about 0.1 mA to about 5 mA, possibly between about 0.1 to about 4 mA, optionally between 0.1 to about 3 mA. Each possibility represents a separate embodiment of the present invention. According to some embodiments, the electric pulse generator is configured to provide sub-threshold electric bursts having a current of between about 0.1 mA to about 5 mA, possibly between about 0.1 to about 4 mA, optionally between 0.1 to about 3 mA. Each possibility represents a separate embodiment of the present invention. According to some embodiments, the voltage (e.g., the pulse generator is configured to provide such a voltage) of the sub-threshold electric bursts is between about 0.1 Volts to about 10 Volts. According to some embodiments, the voltage of the sub-threshold electric bursts is between about 0.1 Volts to about 5 Volts, possibly between about 0.1 Volts to about 3 Volts. Each possibility represents a separate embodiment of the present invention. According to some embodiments, the voltage of the sub-threshold electric bursts is between about 0.1 or 1 Volts to about 10 Volts, possibly between about 3 Volts to about 7 Volts. Each possibility represents a separate embodiment of the present invention.

According to some embodiments, a combination of different sub-threshold electric bursts may be provided according to the systems and methods disclosed herein. According to non-limiting examples, continuous and/or single and/or multiple-discreet sub-threshold electric bursts may be provided alternately, sequentially or according to a pre-determined pattern. Each possibility represents a separate embodiment of the present invention.

According to some embodiments, the system's processor is configured to determine which electric bursts and/or which parameters of electric bursts are to be used according to signals sensed from the subject using at least one sensor.

According to some embodiments, the processor is configured to determine the number of repetitions of sub-threshold electric bursts to provide during each use and/or the type of sub-threshold electric bursts to be used in each repetition. Each possibility represents a separate embodiment of the present invention.

The sub-threshold electric bursts administered according to the systems or methods of some embodiments of the invention may be continuous and/or discrete and have at least part of the characteristics as listed in Table 1 herein below. Each possibility represents a separate embodiment of the present invention. According to some embodiments, each stimulus protocol of sub-threshold electric bursts provided according to the systems and methods disclosed herein may include any number of repetitions of the sub-threshold burst protocols mentioned in Table 1, in any combination of discrete and continuous mode of stimulation.

TABLE 1
Optional sub-threshold electric burst protocols

| | Pulse | Repetitions | Stimula- | | |
|---|---|---|---|---|---|
| | duration | per cycle | tion cycle | Therapy cycle | power |
| Contin- | 100 ms- | 1 | Pulse | 0.5 sec-5 sec | 0.1-5 mA |
| uous | 5 sec | | duration | and/or duration | and/or |
| | | | | of arrhythmia* | 0.05-10 or |
| | | | | | 15 Volt |
| Discrete | 1-10 ms | 10-100 | 10-500 ms | 0.5 sec-5 sec | 0.1-5 mA |
| | | | | and/or duration | and/or |
| | | | | of arrhythmia* | 0.05-10 or |
| | | | | | 15 Volts |

*inter-therapy cycle duration is optionally set according to the persistence and of the arrhythmia and ventricular rate.

According to some embodiments, the applied electric field is or includes a supra-threshold electric field, which is an electric field that results with generation of an action potential in the target cells.

In some exemplary embodiments of the invention, therapy is provided via combined device, for example a pacemaker which also includes electrode (e.g., in CS) as described herein. Optionally, the signals applied are provided by reprogramming an existing circuit (e.g., pacing circuit) rather than provide a dedicated circuit.

### Exemplary Target Tissue

According to some embodiments, the applied electric field generated according to the disclosed system and method is configured to indirectly or directly affect signal propagation through the AV node region, by affecting muscle tissues and/or neural tissues at the vicinity of the AV node. In some embodiments, the electric field is applied directly and/or indirectly to tissues that are able to deliver signals to the AV node, for example the left inferior extension and/or to the right inferior extension of the AV node. In some embodiments the electric field is applied as sub-threshold electric bursts. In some embodiments, the electric field is applied as a combination of sub-threshold electric bursts and supra-threshold electric bursts.

In some embodiments, the electric field is modified according to the target tissue and/or the distance from the target tissue. Distance is a result of implantation and electrode selection (e.g., described herein) and/or anatomy. In some embodiments, the target tissue for electric field application is determined by an expert. In some embodiments, pulse parameters and/or electrode selection can be modified to get desired effect and/or target tissue. In some embodiments, pulse parameters and/or electrode selection can be modified to be within safety parameters and/or to increase efficacy of the applied electric field.

According to some embodiments, electric field application by electrodes placed within the coronary sinus, near a coronary sinus ostium, may be used to directly affect signal propagation through a right AV node extension. Optionally or alternatively, electric field application by electrodes placed within the coronary sinus distally to coronary sinus ostium, may be used to affect signal propagation pathways entering AV node from left atrium.

According to some embodiments, the sub-threshold electric bursts generated according to the disclosed systems and methods are configured to be directed at the autonomic/parasympathetic nervous system, such as, but not limited to, the parasympathetic (PS) ganglion plexi of the heart. According to some embodiments, the PS ganglion plexi of the heart are selected from the group consisting of: ganglia in the fat pad of the superior cavo-atrial junction, ganglia in the fat pad of the inferior cavo-atrial junction and a combination thereof. Each possibility represents a separate embodiment of the present invention. According to some embodiments, at least part of the electrodes according to the systems and methods of the present invention are configured (e.g., selected and/or electrification parameters thereof selected) to deliver sub-threshold electric bursts to PS ganglion plexi of the heart. According to some embodiments, at least one of the electric-pulse generator and/or the processor of the disclosed system is configured to enable delivery of sub-threshold electric bursts to PS ganglion plexi of the heart. According to some embodiments, at least part of the electrodes according to the systems and methods of the present invention are configured to be situated at or near the subject's PS ganglion plexi of the heart. According to some embodiments, the plurality of electrodes according to the systems and methods of the present invention are configured to be situated at or near the subject's PS ganglion plexi of the heart.

According to some embodiments, delivery of sub-threshold electric bursts to the subject's atrioventricular (AV) node refers to delivery of sub-threshold electric bursts to parasympathetic ganglion plexi of the heart. According to some embodiments, electrodes configured to deliver sub-threshold electric bursts to the subject's atrioventricular (AV) node are electrodes configured to deliver sub-threshold electric bursts to parasympathetic ganglion plexi of the subject's heart.

According to some embodiments, sub-threshold electric bursts configured to be directed at parasympathetic ganglia in the heart may have a current of about 0.5-13 mA, preferably about 1.5-10 mA. Each possibility represents a separate embodiment of the present invention. According to some embodiments, sub-threshold electric bursts configured to be directed at parasympathetic ganglia in the heart may have a burst duration of about 40-60 ms, preferably about 50 ms. According to some embodiments, sub-threshold electric bursts configured to be directed at parasympathetic ganglia in the heart may be provided at rectangular 10 second bursts at a frequency of 20 Hz and a pulse duration of about 0.05 ms, wherein the voltage ranges about 1-20 V. According to some embodiments, sub-threshold electric bursts configured to be directed at parasympathetic ganglia in the heart may have discrete burst duration of about 0.1-0.5 ms at about 1-10V and a frequency of about 50 Hz. According to some embodiments, sub-threshold electric bursts configured to be directed at parasympathetic ganglia in the heart may have an amplitude of about 10V, a pulse duration of about 1 ms, biphasic waveform (95% positive and 5% negative), a pulse-to-pulse interval of about 20 ms (i.e., pulse rate 50 Hz), a burst duration of 180 seconds (10 pulses), and 90 bursts/minute.

In some exemplary embodiments of the invention, the placement (and localization) of electrodes and of stimulation may be important for a variety of reasons, including, a desire to avoid stimulating non-target tissue, a desire to reduce energy and power requirements, small size of the AV node and/or target associated tissue and/or existence of nonconducting tissue.

### Exemplary System

An aspect of some embodiments of the present invention relates to at least one electrode combined with at least one additional component, placed within the coronary sinus and configured to apply an electric field to affect signal propagation between the atria and ventricles. The additional component is optionally selected from a list consisting of at least one pulse generator, at least one power source, and at least one processor (e.g., as a non-limiting example of control circuitry).

In some embodiments electrodes are positioned in the proximal part of the coronary sinus in locations that are in close vicinity to signal conduction pathways. In some embodiments electrodes are positioned in locations with high probability to affect signal conduction between atria and ventricles. In some embodiments electrodes and at least one additional element are placed within and/or on and/or extending from a surface of a casing configured to be inserted into the coronary sinus. Additionally, in some embodiments, the casing comprises at least one anchoring element configured to anchor the casing at least partly within the coronary sinus.

According to some embodiments, the system of the present invention comprises at least one electrode, possibly incorporated into or attached to a stent configured to be inserted into the coronary sinus, the at least one electrode is configured to direct sub-threshold electric bursts at parasympathetic ganglia in the heart. Each possibility represents a separate embodiment of the present invention. According to some embodiments, all the components of the disclosed system are comprised in and/or are integrally formed with and/or are attached to the stent configured to be inserted into the coronary sinus, such that the implantable electrical stimulation system of the invention is lead-less. Each possibility represents a separate embodiment of the present invention. According to some embodiments, the implantable electrical stimulation system of the invention which is comprised in and/or incorporated with a stent configured to be inserted into the coronary sinus, further comprises an energy source, such as, but not limited to, a battery, such that the entire system is implantable in the coronary sinus without need of an external energy source.

According to some embodiments, the disclosed system comprises at least one electric pulse generator. According to some embodiments, each electrode in the plurality of electrodes is functionally connected to at least one electric pulse generator. According to some embodiments, at least one electrode in the plurality of electrodes is integrally formed with at least one electric pulse generator. According to some embodiments, each electrode in the plurality of electrodes is functionally connected to an electric pulse generator. According to some embodiments, at least part of the plurality of electrodes is integrally formed with at least one electric pulse generator.

According to some embodiments, the plurality of electrodes is integrally formed with, attached to and/or is part of an anchoring element configured to anchor the electrodes and/or implantable device within or partially within the coronary sinus.

According to some embodiments, the plurality of electrodes is integrally formed with, attached to or is part of a stent configured to be inserted into the coronary sinus, wherein the plurality of electrodes is functionally connected to and possibly integrally formed with at least one electric pulse generator. Each possibility represents a separate embodiment of the present invention.

According to some embodiments, each electrode of the plurality of electrodes is functionally connected with at least one electric pulse generator. As used herein, an electrode functionally connected to an electric pulse generator refers to an electrode coupled to the pulse generator such that a sub-threshold burst generated by the pulse generator can be transmitted by the electrode. According to some embodiments, at least part of the electrodes are connected to the electric pulse generator through at least one lead, such as, but not limited to, a metal lead or a lead made of an electricity-conducting material.

According to some embodiments, electrodes which may be used to deliver a sub-threshold electrical burst and/or other electric fields according to some embodiments of the invention are any electrodes capable of delivering electrical current to the heart, such as, but not limited to, electrodes used with artificial pacemakers. It is to be noted that the term “electrode” is used herein for brevity but any electricity conducting means which is able to deliver a sub-threshold electric pulse to the AV node may be used with the system of the invention. According to some embodiments, delivering a sub-threshold electric burst to the AV node refers to delivering a sub-threshold electric burst which prolongs the effective refractory period of myocytes in the AV node.

In some exemplary embodiments of the invention, a same electrode is used both for stimulation and for sensing electrical activity (e.g., with a switch between connections to power and sensing circuitry). Optionally or alternatively, a stimulation electrode is used for ablation. For example, if stimulation suggests that ablation will provide therapeutic benefit, the anchoring of the electrodes may be used to simplify an ablation procedure by ablating via one or more electrodes already implanted.

According to some embodiments, the electrodes comprise a proximal end configured to be functionally connected to at least one electrical-pulse generator and a distal end configured to be positioned at or proximal to the AV node of the subject's heart. According to some embodiments, the proximal ends of the electrodes are attached to at least one electric-pulse generator through at least one lead, such as, but not limited to, a metal lead or any lead capable of conducting electricity. According to other embodiments, the electrodes are at least partly integrally formed with the electric pulse generator.

According to some embodiments, at least part of the plurality of electrodes within the disclosed system are leadless electrodes. According to some embodiments, leadless electrodes are electrodes configured to wirelessly communicate with the system's processor and receive signals to actuate or arrest delivery of sub-threshold electric current. Each possibility represents a separate embodiment of the present invention. According to some embodiments, the leadless electrodes are at least partly integrally formed with at least one electric pulse generator.

According to some embodiments, the distal ends of at least some of the plurality of electrodes are incorporated into an at least partially conducting surface positioned at the AV node. According to some embodiments, at least some of the plurality of electrodes are incorporated into an at least partially conducting surface positioned at the AV node. According to some embodiments, at least the selected subset of electrodes is configured to deliver sub-threshold electric bursts to the AV node of the subject and deliver substantially no electric current to other areas of the heart.

According to some embodiments, the distal ends of at least some of the electrodes are incorporated in or integrally formed with a stent configured to be positioned in the coronary sinus. Each possibility represents a separate embodiment of the present invention.

A particular feature of some embodiments of the invention is that a plurality of electrodes may be able to stimulate a same target region (e.g., AV node portion and/or AV node extension portion). However, one electrode may require less power, have fewer side effects and/or be more reliable than another electrode. In some exemplary embodiments of the invention, the electrodes are spaced so as to increase the changes that at least one electrode is suitable (or several if several regions are to be stimulated together), optionally that there are at least two or three such electrode. Such spacing may be determined, for example, based on anatomical consideration, anatomical variations and/or variation in implantation quality.

In some exemplary embodiments of the invention, the electrodes are spaced apart, for example, 1 mm, 2 mm, 3 mm or smaller or intermediate distances. Optionally, the spacing is uniform. In some embodiments, the spacing is non-uniform, for example, a smaller spacing provided in areas where there is a higher likelihood of side effects of stimulation. Optionally or alternatively, to axial spacing, circumferential spacing may be provided, for example, 20 degrees, 40 degrees, 90 degrees, 120 degrees, 180 degrees and smaller, intermediate and/or larger spacings. In some cases (e.g., helical designs) spacing may be simultaneously axial (and/or along the surface of the CS) and angular.

In some exemplary embodiments of the invention, even at a same axial location, multiple angular locations may be provided, for example, 2, 3, 4, 5 or larger numbers of electrodes, each aimed in a different direction.

In some exemplary embodiments of the invention, the electrodes and/or anchoring structures include one or more radio-opaque markers, for example, between 2 and 5 markers, to allow an orientation of the array to be detected using x-ray imaging.

In some exemplary embodiments of the invention, at least some of the electrodes are shaped and sized so that the volume effectively stimulated by an electrode is generally pyramidal (or conical) and has an apex angle (even if truncated) of between 10 and 90 or 120 degrees.

In some exemplary embodiments of the invention, target tissue can be localized by the electrode (e.g., and power settings) to volumes of, for example, between 0.01 and 3 cubic centimeters, for example between 0.1 and 1 cubic cm. Optionally, a target tissue region is in the shape of a pyramid extending away from a CS to a distance of between 1 and 10 mm, for example, between 2 and 7 mm.

In some exemplary embodiments of the invention, an overlap between two nearby electrode is between 10%-20%, 20%-50% and/or 50%-70% in stimulated volume (e.g., with a same parameter as used for therapy).

In some exemplary embodiments of the invention, the shape of stimulated volume is controlled by using two electrodes within the CS or using one electrode in the CS and one outside the CS (e.g., in the heart and/or the can of the device) and/or outside the body.

In some exemplary embodiments of the invention, an electrode has an area of between 0.1 mm square and 40 mm square, for example, between 1 and 10 mm square. Optionally, an electrode is generally rectangular or circular with a maximal extent of between 0.1 and 4 mm, for example, between 0.5 and 2 mm. In some exemplary embodiments of the invention, an electrode is ring shaped with a width of, for example, between 0.1 and 5 mm.

In some exemplary embodiments of the invention, an axial extent of electrodes (along which electrodes are located) is between 1 and 70 mm, for example, between 2 and 10 mm or between 4 and 30 mm.

In some exemplary embodiments of the invention, stimulating at or near the ostium is of particular interest. Optionally, the electrodes are designed to extend outwards sufficiently to contact the ostium, for example at a point of inflection in the curvature of the atrial-CS junction.

In some exemplary embodiments of the invention, the electrodes are mounted on a tubular anchoring structure and the tube is configured (e.g., to self expand or be balloon expandable) so it can flare out (and/or include one or more flaring extensions) and contact the ostium and/or tissue adjacent the ostium (e.g., between 0.1 and 34 mm past the ostium onto the atrial wall.

In some exemplary embodiments of the invention, only a single region is stimulated at a time, for example, using one or more electrodes (e.g., two nearby electrodes may be used to, together, cover a larger area to be stimulated). In some exemplary embodiments of the invention, however, multiple, disjoint regions are to be stimulated. In one example, the AV node and two of its extensions are to be stimulated. Optionally, such disjoint (or other multiple-electrode) stimulation may include simultaneous application of an electric field at multiple electrodes. Optionally or alternatively, the electrification may be sequential, for example, one electrode being electrified, while another is between electrifications. This may allow the use of lower cost and/or smaller power circuitry.

According to some embodiments, electrodes are configured to deliver an electric field to a tissue by placing the electrodes in contact with the tissue. In some embodiments, the distance between two adjacent electrodes is at least 1 mm. In some embodiments electrodes are point electrodes configured to apply an electric field and/or current to a single location in the tissue. In some embodiments, electrodes are ring electrodes having a circular structure configured to be placed within a blood vessel, and apply an electric field to multiple locations simultaneously on the blood vessel wall. In some embodiments, plurality of electrodes are both point and ring electrodes.

In some exemplary embodiments of the invention, the electrodes are sized and shaped to avoid damaging the wall of the CS when pressed against and/or to penetrate a short distance thereto without tearing of the wall (e.g., include short spikes backed by pads.

In some exemplary embodiments of the invention, a system includes between 2 and 40 separately electrifiable electrodes, for example, between 2 and 20 or between 4 and 15.

Optionally, each electrode has its own electrification wire. Optionally, the wires are coupled to a flat braid or to a flat flexible PCB.

In some exemplary embodiments of the invention, one or more non-CS electrodes are provided. Optionally, such an electrode includes a tissue attachment tip, for example a suture, a screw, a clip or other means, such as may be known in the art.

According to some embodiments, electrodes comprise a conductive material configured to deliver sub-threshold and supra-threshold electric bursts. In some embodiments, electrodes comprise a bio-inert material, for example titanium, gold, silver, platinum, and any combination hereof.

According to some embodiments, at least some of the electrodes are incorporated in or integrally formed with an anchoring element configured to position and anchor the electrodes and/or implantable system within or partially within a blood vessel, for example the coronary sinus. In some embodiments the anchoring element is a hook, configured to fixate the electrode to the surface of the blood vessel wall facing the lumen. Alternatively, the anchoring element is a pin connected to the electrode, configured to fixate the electrode to the surface of the blood vessel wall facing the lumen. Optionally, the anchoring element is formed from a perforated mesh or grid comprising electrodes. Mesh or grid are optionally configured to bend and form a radial shaped structure. The radial shaped structure is configured to allow the flow through of fluid when electrodes and anchoring element are anchored within a blood vessel lumen.

In some embodiments anchoring element comprise a bio-inert material, for example titanium, gold, silver, platinum, and any combination hereof. In some embodiments an anchoring component may comprise a bioresorbable metal, for example iron and/or magnesium. In some embodiments, anchoring element comprise a shape memory metal, for example nitinol. In some embodiments, anchoring element is a radial structure configured to support the structure of a blood vessel by applying positive pressure against the inner surface of the blood vessel wall. In some embodiments electrodes are located on the exterior surface of the anchoring element, facing blood vessel wall.

In some embodiments, anchoring element may comprise a coating applied to at least part of its interior surface, its exterior surface, or both. Coating further comprising a sustained release formulation of pharmaceutical compounds configured to reduce blood vessel occlusions. In some embodiments, anchoring element may comprise a coating applied to at least part of its exterior surface facing the inner side of the blood vessel wall. Exterior surface coating further comprising a formulation of chemical and/or biological compounds configured to increase the attachment and contact of the anchoring element to the blood vessel wall.

According to some embodiments, the electrodes comprised in the anchoring element according to the invention are leadless electrodes. According to some embodiments, the electrodes comprised in the anchoring element according to the invention are at least partly attached to or integrally formed with at least one of the following additional elements: at least one pulse generator, at least one power source, or a processor. In some embodiments electrodes positioned within the coronary sinus, such as electrodes comprised in an anchoring element, are able to apply an electric field to affect signal propagation through the AV node of the subject due to the proximity between the coronary sinus and AV node. Accordingly, positioning electrodes incorporated into an anchoring element within the coronary sinus may obviate the need to further fix electrodes to the AV node itself.

According to some embodiments, at least some of the electrodes are incorporated in or integrally formed with a stent configured to be positioned in the coronary sinus. Each possibility represents a separate embodiment of the present invention. According to some embodiments, the disclosed system comprises a stent configured to be positioned in the coronary sinus, wherein at least part of the stent comprises the plurality of electrodes. According to some embodiments, the electrodes comprised in the stent according to the invention are leadless electrodes. According to some embodiments, the electrodes comprised in the stent according to the invention are at least partly attached to or integrally formed with at least one electric pulse generator. Without wishing to be necessarily bound by any theory or mechanism, electrodes positioned within the coronary sinus, such as electrodes comprised in a stent, are able to deliver sub-threshold electrical bursts to the AV node of the subject due the proximity between the coronary sinus and AV node. Accordingly, positioning electrodes incorporated into a stent within the coronary sinus may obviate the need to further fix electrodes to the AV node itself.

According to some embodiments, the system's electrodes, electric pulse generator, sensors and processor are at least partly attached to or integrally formed with a stent configured to be positioned within the coronary sinus. Without wishing to be necessarily bound by any theory or mechanism, positioning an implantable electrical stimulation system as disclosed herein in a coronary sinus, all essential parts of which substantially comprised or attached to at least one stent, enables easy insertion of the system, easy fixation to the coronary sinus and accurate delivery of sub-threshold electric bursts by the system.

It is noted that in accordance with some embodiments of the invention a device for implanting partly or wholly within a CS may also be used (instead or in addition) for treatments other than modifying AV node conductance, for example, for pacing.

According to some embodiments, the disclosed system is configured to be at least partially inserted into the coronary sinus of a subject's heart. According to some embodiments, the disclosed system is configured to be entirely inserted into the coronary sinus of a subject's heart. According to some embodiments, the disclosed system is a leadless system configured to be entirely inserted into the coronary sinus of a subject's heart. According to some embodiments, the disclosed system comprises at least one stent configured to be positioned within the coronary sinus of the subject's heart. According to some embodiments, the disclosed system comprises at least two stents configured to be positioned within the coronary sinus of the subject's heart. According to some embodiments, the disclosed system is configured to be entirely inserted into the coronary sinus of a subject's heart an comprises a stent in the proximal end of the system (the side closest to the proximal end of the coronary sinus) and/or a stent in the distal end of the system (the side closest to the distal end of the coronary sinus). Each possibility represents a separate embodiment of the present invention.

According to some embodiments, the stent in the proximal end of the system and/or the stent in the distal end of the system comprise at least one electrode and/or at least one heart-activity sensor. Each possibility represents a separate embodiment of the present invention. According to some embodiments, the stent in the proximal end of the system and/or the stent in the distal end of the system comprise a plurality of electrodes and/or at least one heart-activity sensor. Each possibility represents a separate embodiment of the present invention. According to some embodiments, the stent in the proximal end of the system comprises a plurality of electrodes configured to provide sub-threshold electric bursts to the subject's AV node.

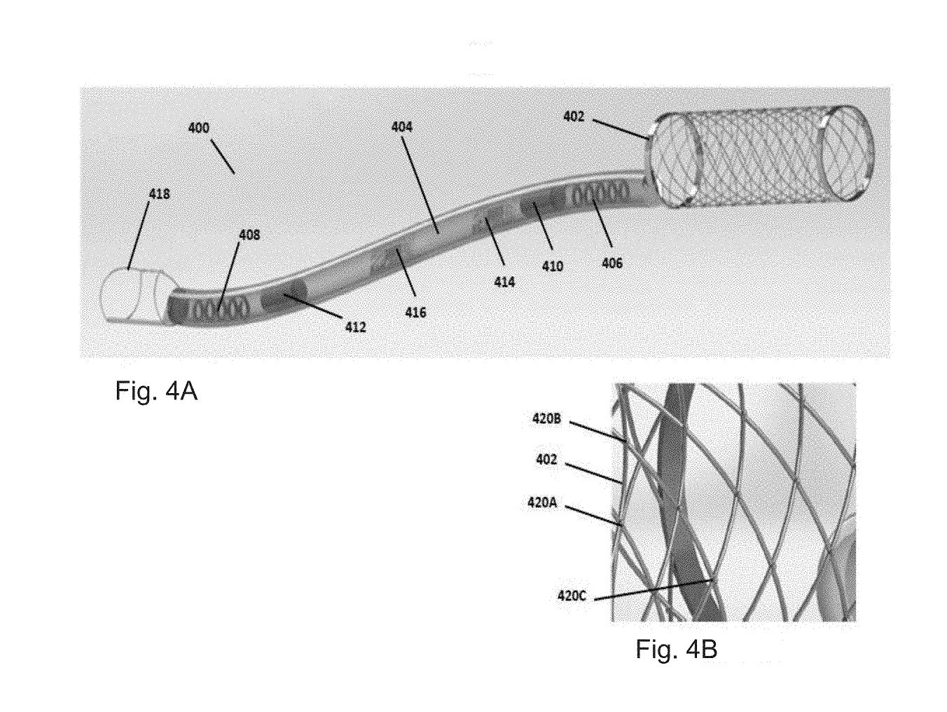

As used herein, the term “stent” loosely refers to an elongated tube-like structure configured to be inserted to a blood vessel, specifically at least to a part of the coronary sinus. It is noted that in some embodiments, such a stent actually has a conical configuration and/or is flared or flarable to match an ostium geometry. In any case, a function of supporting a CS against surrounding tissue and/or stenosis may be omitted. According to some embodiments, a stent comprises an open conformation and a closed conformation. According to some embodiments, the closed conformation is a compact form which enables easy insertion of the stent into a blood vessel using common means, such as, but not limited to, a catheter. According to some embodiments, the open conformation enables firm fixation of the stent within the coronary sinus, thus enabling delivery of sub-threshold electric bursts to the AV node through electrodes comprised in or attached to the stent. Each possibility represents a separate embodiment of the present invention. According to some embodiments, when in the open position, the stent according to the invention is configured to have a length and diameter which enable firm fixation within the coronary sinus. According to some embodiments, the length and width of the stent in the open position do not exceed the average internal length and width of the coronary sinus. According to some embodiments, a stent may have several sections, each section having the same or different volume when in the open conformation. A non-limiting example of such a stent is depicted in FIG. 4.

Of note, the disclosed system may provide only sub-threshold electric bursts to the AV node of a subject and thus may not require ablation of the AV node which is a prerequisite for use of common artificial pacemakers. Artificial pacemakers which require ablation of the AV node are known in the art and include, but are not limited to, implantable and intra-cardial pacemakers.

According to some embodiments, the sub-threshold electric bursts are delivered from at least two electrodes either simultaneously, subsequently or in an overlapping fashion. Each possibility represents a separate embodiment of the present invention. According to some embodiments, the system's processor is further configured to induce delivery of the sub-threshold electrical bursts from at least two electrodes either simultaneously, subsequently or in an overlapping fashion. Each possibility represents a separate embodiment of the present invention. According to some embodiments, sub-threshold electrical bursts delivered from different electrodes may have at least one differing characteristic, such as, but not limited to, current, frequency and timing. Each possibility represents a separate embodiment of the present invention.

According to some embodiments, the system comprises at least one heart-activity sensor. According to some embodiments, the at least one heart-activity sensor is configured to sense atrial activity and/or ventricular activity. Each possibility represents a separate embodiment of the present invention. According to some embodiments, the at least one heart-activity sensor is configured to sense both atrial activity and ventricular activity.

According to some embodiments, the at least one heart-activity sensor is selected from the group consisting of: an atrial sensor, a ventricular sensor and a combination thereof. Each possibility represents a separate embodiment of the present invention. According to some embodiments, the at least one heart-activity sensor is configured to sense near-field and/or far-field electric activity. Each possibility represents a separate embodiment of the present invention. According to some embodiments, the at least one heart activity sensor is able to sense both atrial activity and ventricular activity.

According to some embodiments, the system comprises at least one atrial sensor configured to sense atrial activity. According to some embodiments, the system comprises at least one ventricular sensor configured to sense ventricular activity. According to some embodiments, the system comprises at least one atrial sensor configured to sense atrial activity and at least one ventricular sensor configured to sense ventricular activity.

According to some embodiments, the atrial activity is selected from the group consisting of: atrial contraction, atrial rate, atrial depolarization, atrial repolarization and a combination thereof. Each possibility represents a separate embodiment of the present invention. According to some embodiments, the ventricular activity is selected from the group consisting of: ventricular contraction, ventricular rate, ventricular depolarization, ventricular repolarization and a combination thereof. Each possibility represents a separate embodiment of the present invention.

Without being limited to the following list, an example sensor may be an electrical activity sensor (e.g., unipolar or bipolar), an impedance sensor, a force sensor, a blood pressure sensor, a blood flow sensor, an accelerometer and/or other sensors as may be known in the art of cardiac stimulation and/or feedback systems. As noted herein, a plurality of sensors, for example, 2, 3, 4 or more may be provided. Optionally or alternatively, a same electrode may be used both for sensing and for electrification and/or may be usable therefore.

According to some embodiments heart-activity sensor comprise a conductive material configured to deliver electric current. In some embodiments, heart activity sensors comprise a bio-inert material, for example titanium, gold, silver, platinum, and any combination hereof. In some embodiments sensor signals the processor using lead wiring. Alternatively, sensor signals the processor by wireless means. In some embodiments heart activity sensors are incorporated or integrally formed with an anchoring element configured to attach the sensor to a tissue, for example blood vessel wall or fat pad.

Exemplary Treatment

According to some embodiments, the sensors of the disclosed system are configured to transfer sensed input to the system's processor. According to some embodiments, the system's processor is configured to determine, based on input from at least one heart activity sensor, whether the subject is undergoing atrial fibrillation.

According to some embodiments, the system's processor is configured to determine, at least based on the at least one ventricular sensor, whether the subject is undergoing atrial fibrillation. According to some embodiments, the system's processor is configured to determine, based on input from at least one atrial sensor, whether the subject is undergoing atrial fibrillation. According to some embodiments, the system's processor is configured to determine, based on input from at least one atrial sensor and at least one ventricular sensor, whether the subject is undergoing atrial fibrillation. According to some embodiments, the system's processor is configured to actuate delivery of sub-threshold electric bursts to the AV node of a subject only if the subject is undergoing an episode of atrial fibrillation. According to some embodiments, the system's processor is configured to actuate delivery of sub-threshold electric bursts to the AV node of a subject only if the subject is undergoing an episode of atrial fibrillation and has a ventricular rate above a pre-determined range, such as, but not limited to, above 100 Beats Per Minute.

According to some embodiments, the system's processor is configured to determine whether a subject is undergoing atrial fibrillation based on at least one of: frequency of atrial depolarization, atrial contraction rate, frequency of ventricular depolarization, ventricular contraction rate and any combination thereof. Each possibility represents a separate embodiment of the present invention. According to some embodiments, the system's processor is configured to determine whether a subject is undergoing atrial fibrillation based on input received from at least one heart-activity sensor.

According to some embodiments, the system's processor is configured to determine whether a subject is undergoing atrial fibrillation and/or initiate delivery of sub-threshold electric bursts to the subject's AV node using an Automatic Mode Switching (AMS) algorithm. Each possibility represents a separate embodiment of the present invention. Non-limiting examples of AMS algorithms which may be used according to the present invention were reviewed in a paper by Stabile et al. (Indian Pacing Electrophysiol. J. 2005 July-September; 5(3): 186-196), the contents of which are incorporated herein in their entirety.

According to some embodiments, the system's processor is configured to determine that a subject is undergoing atrial fibrillation if input accumulated from the at least one heart-activity sensor indicates that atrial rate exceeds a programmable “cut-off” value (for a defined period of time or cycles). According to other embodiments, the system's processor is configured to determine that a subject is undergoing atrial fibrillation if the duration of the mean atrial cycle as sensed by the at least one heart-activity sensor shortens to a predetermined duration. According to certain embodiments, the processor is configured to determine the “physiological” sinus rhythm of the patient based on input obtained from the at least one heart-activity sensor and, taking into account the fluctuation in sinus rate, the processor may identify rates beyond the upper range as atrial fibrillation.

According to some embodiments, the system's processor is configured to allow delivery of sub-threshold electric bursts only in a subject undergoing atrial fibrillation.

According to some embodiments, the system's processor is configured to allow delivery of sub-threshold electric bursts only if the ventricular rate, as sensed by the at least one ventricular sensor, does not correspond with a pre-determined range. According to some embodiments, the system's processor is configured to allow delivery of sub-threshold electric bursts only if the ventricular rate, as sensed by the at least one ventricular sensor, is higher than a pre-determined range. According to some embodiments, the system's processor is configured to actuate delivery of sub-threshold electric bursts only in a subject undergoing atrial fibrillation and having a ventricular rate higher than a pre-determined range. According to some embodiments, the system's processor is configured to allow delivery of sub-threshold electric bursts only through the selected subset of one or more electrodes which was determined by the processor to be able to deliver the bursts to the subject's AV node.

According to some embodiments, a pre-determined range relates to a range of ventricular rates which are considered normal for a healthy individual of the same age, sex and physiological characteristics as the subject. According to some embodiments, a pre-determined range relates to an average range of ventricular rates which are considered normal for healthy subjects of various ages and physiological conditions. According to some embodiments, a ventricular rate which is higher than the pre-determined ventricular rate is indicative of tachycardia. According to some embodiments, a ventricular rate having a frequency different than the pre-determined rate may indicate an arrhythmia such as atrial fibrillation. According to some embodiments, atrial fibrillation may induce ventricular rate which is higher than normal and/or has irregular frequency, thus resulting in a ventricular rate which does not correspond to the pre-determined range.

As used herein, the term ventricular rate refers to the number of ventricle contractions per time unit. Normal ventricular rate may be within 60-100 beats per minute at rest. According to some embodiments, normal ventricular rate has a regular frequency. According to some embodiments, the pre-determined range is between 60-100 beats per minute. According to some embodiments, the pre-determined range is a range of ventricular rates between 60-100 beats per minute. According to some embodiments, the pre-determined range is about 100 BPM. According to some embodiments, the pre-determined range is about 90-110 BPM.

According to some embodiments, the pre-determined range of ventricular rates is determined by a medical professional according to parameters correlated to physiological characteristics of the subject. According to other embodiments, the pre-determined range of ventricular rates is calculated by the system's processor. According to other embodiments, the pre-determined range of ventricular rates is pre-programmed to the system's processor. According to other embodiments, the pre-determined range of ventricular rates is calculated by the system's processor according to the normal ventricular rate of the subject as measured by the at least one heart-activity sensor while the subject was not undergoing atrial fibrillation.

According to some embodiments, the processor is configured to measure the ventricular rate of the subject using at least part of the input received from the at least one heart-activity sensor. According to some embodiments, the processor is configured to determine whether the ventricular rate is within, lower than or higher than the pre-determined range. According to some embodiments, in order for the system's processor to determine if the ventricular rate of the subject is above the pre-determined range, the ventricular rate is measured for at least 30 seconds, preferably at least 1 minute. Each possibility represents a separate embodiment of the present invention. According to some embodiments, the ventricular rate is determined by the system's processor using input received from the at least one heart-activity sensor. According to some embodiments, the system's processor is configured to determine whether the ventricular rate is regular. According to some embodiments, a ventricular rate which is regular and within the pre-determined range is a ventricular rate which is not affected by atrial fibrillation. According to some embodiments, the system's processor is configured to actuate delivery of sub-threshold electric bursts to the subject's AV node if the subject is undergoing atrial fibrillation and has a ventricular rate above a pre-determined range, such as, but not limited to, above 100 Beats Per Minute. According to some embodiments, the processor of the disclosed system is configured to induce arrest of sub-threshold electric bursts delivery to the subject's AV node. According to some embodiments, the processor of the disclosed system is configured to induce arrest of sub-threshold electric bursts delivery to the subject's AV node when the subject is no longer undergoing atrial fibrillation and has a ventricular rate below or within the pre-determined range, such as, but not limited to, about 100 BPM. According to some embodiments, arrest of delivery of sub-threshold electric bursts according to the disclosed systems and methods is performed gradually. According to some embodiments, arrest of delivery of sub-threshold electric bursts according to the disclosed systems and methods is performed gradually with stimulation frequency decreasing in a predetermined manner. According to some embodiments, during arrest of delivery of sub-threshold electric bursts to the subject's AV node, the stimulation frequency decreases gradually corresponding to the decrease of ventricular rate towards the pre-determined range.

Electrode Selection/Mapping

According to some embodiments, the system's processor is configured to select an electrode or set of electrodes which is able to deliver an electric field to affect signal propagation between the atria and ventricles. In some embodiments, the processor signals to apply an electric field through an electrode or electrode set located proximal to the coronary sinus ostium (e.g., inside the CS). In some embodiments, electrode selection and/or mapping include (also and/or only) electrodes that are outside the CS and/or which contact the CS ostium itself. Following electric field application, processor receives heart activity parameters from heart activity sensors and determines whether the applied electric field resulted with a desired effect. In some embodiments, a desired effect can be reducing the number of ventricle contractions and/or increasing the number of ventricle contractions. If the desired effect was reached, then the processor needs to decide whether to apply a second electric field through the same electrode or electrode set shown to be efficacious, or to continue the mapping process and apply a second electric field through an adjacent and more distal electrode or electrode set, followed by further analysis of the electric field application result. Processor is configured to decide whether to select an efficacious electrode or to continue mapping the efficacy of other electrodes is based on pre-determined parameters stored within the system.

In some embodiments, the electric field is applied as sub-threshold electric bursts or as supra-threshold electric bursts and/or as a combination of both sub-threshold and supra-threshold electric bursts.

According to some embodiments, the system's processor is configured to select a subset of the plurality of electrodes which is able to deliver sub-threshold electric bursts to the subject's AV node. According to some embodiments, the system's processor is configured to select a subset of the plurality of electrodes which is able to deliver sub-threshold electric bursts to the subject's AV node such that the bursts induce a ventricular rate within a pre-determined range. In some embodiments, the term “subset” refers to one or more, possibly at least two and/or possibly the whole set. Each possibility represents a separate embodiment of the present invention. According to some embodiments, the system's processor is configured to actuate delivery of sub-threshold electric bursts only through the selected subset of electrodes.

It is noted that while in some embodiments the mapping is managed using an implanted processor, in other embodiments an external processor and/or programmer control the process. Optionally, the external processor is connected directly to the electrodes. Optionally or alternatively, the external processor sends commands to the implanted processor.

According to some embodiments, the system's processor is configured to determine which subset of the plurality of electrodes is able to deliver sub-threshold electric bursts to the subject's AV node. According to some embodiments, the system's processor is configured to determine which subset of the plurality of electrodes is able to deliver sub-threshold electric bursts to the subject's AV node such that the bursts induce a ventricular rate within the pre-determined range. According to some embodiments, the system's processor is configured to determine which of the plurality of electrodes or combination thereof is positioned such that it is able to deliver sub-threshold electric bursts to the subject's AV node thus inducing a ventricular rate within the pre-determined range.

According to some embodiments, a ventricular rate within the pre-determined range is a ventricular rate which is not affected (and/or otherwise appears normal) by atrial fibrillation signals.

According to some embodiments, a subset of one or more of the plurality of electrodes refers to a combination of electrodes. According to some embodiments, the system's processor is configured to determine which combination of electrodes is able to deliver sub-threshold electric bursts to the subject's AV node such that the bursts induce a ventricular rate within the pre-determined range. According to some embodiments, the combination of electrodes is a combination of at least two electrodes, preferably at least three electrodes. Each possibility represents a separate embodiment of the present invention.

While in some embodiments of the invention mapping relates to electrode selection, optionally or alternatively other parameters are searched for. For example, a desired amplitude may be searched for.

While in some embodiments search focuses on efficacy. Other targets may be used as well or instead. For example, the search may be selected to reduce power needs and/or to avoid side effects and/or enhance safety. In each case, a search method may include testing a first set of parameters (e.g., electrodes and/or signal settings), checking an effect and repeating with another setting and/or selecting a previously found setting. Optionally, a further step of optimizing may be applied after an initial setting is found.

In some exemplary embodiments of the invention, mapping is used to compensate for an unknown orientation and/or axial position and/or contact quality of electrodes in or near a CS. For example, if non-ring electrodes are used, mapping may be used to select (e.g., by checking electrodes at different circumferential positions by perhaps same or close axial positions) which electrode is aimed at a target tissue and/or avoid an electrode aimed at non-target tissue.

Exemplary Application Protocol

According to some embodiments, heart activity sensors are configured to transfer sensed input to the system's processor. The system's processor is configured to analyze the sensed input, and compare the analyzed input to pre-determined parameters. The processor is configured to determine based on the comparison results, the current clinical state of the subject. In some embodiments, pre-determined parameters are stored in a storage element functionally connected to the processor. Once the clinical state was determined, the processor is configured to decide whether to apply an electric field by the implantable system. In some embodiments, the processor is configured to match an application protocol to the determined clinical state. Application protocols include at least one electric field application parameter selected from a list of application process duration, electric field voltage, electric field current, electric field frequency and electric field type for example a sub-threshold electric field or a supra-threshold electric field. In some embodiments, application protocols are stored in a storage element functionally connected to the processor. Once an application protocol is matched to the current clinical state, the processor signals the pulse generator to generate electric pulses and to deliver the pulses to the tissue through electrodes. The processor continues to receive heart activity parameters from heart activity sensors, and after re-analysis, to determine whether the applied electric field resulted with the desired effect. In some embodiments, if the desired effect was not reached then a sequential electric field is applied according to the previous application protocol through a different electrode or electrode set. Alternatively, the processor chooses a different application protocol, and signals pulse generator to generate pulses according to application parameters detailed in the new application protocol.

According to some embodiments, the system's processor is further configured to determine which parameters of sub-threshold electric bursts, such as, but not limited to, current, voltage and frequency, are required in the selected electrode subset or combination thereof in order to induce a ventricular rate within the pre-determined range. According to a non-limiting example, the system's processor may determine that a combination of two electrodes is required to deliver sub-threshold electric bursts to the subject's AV node and may further determine the current and/or voltage and/or frequency of bursts for each of the electrodes. According to some embodiments, each of the electrodes in the plurality of electrodes is configured to provide the same or different sub-threshold electric bursts. Each possibility represents a separate embodiment of the present invention. According to some embodiments, the processor is configured to induce delivery of sub-threshold electric bursts having similar or different parameters through each electrode in the selected subset. Each possibility represents a separate embodiment of the present invention.

In some exemplary embodiments of the invention, determination is based on a preprogrammed table or logic. For example, one or more of the following examples (and/or two or three or more in combination) may be provided as a table (e.g., mapping sensed and/or desired values to one or more signal parameters) and/or as logic (e.g., defining values and thresholds for decision making. In any case, some parameters may be preset and/or found by search.

In some exemplary embodiments of the invention, cardiac demand (e.g., sensed using an accelerometer and/or other means, for example as known in the art) is used as an indication of a desired ventricular rate, for example, increased demand suggesting an increased ventricular rate. For example, an accelerometer indicating movement associated with walking may cause one ventricular rate (or range) to be desired and a pattern indicating running, cause a higher such rate to be desired and set as a target by the processor logic.

In some exemplary embodiments of the invention, sensing is used to detect an atrial arrhythmia and signals applied in anticipation of a negative effect thereof on ventricular rate.

In some exemplary embodiments of the invention, sensing is used to detect a ventricular state and if it is determined the state is affected by an atria (e.g., based on a detection of ectopic beats, rate, heart rate variability, change in heart rate, morphology of change in rate, abnormality of ECG and/or other indications of arrhythmia and/or suboptimal activation), a signal applied at or near the AV node, optionally using the ventricular sensing as feedback.

In some exemplary embodiments of the invention, AT (atrial tachycardia) is detected and the timing of the signals are applied so as to allow the atrial activity to be synchronized at a fixed phase (optionally) to the ventricular activity.

In some exemplary embodiments of the invention, reentrant AV node arrhythmia are detected and the signals used to stop it.

In some exemplary embodiments of the invention, AV block is detected and the signals applied to at least transiently unblock the AV node.

In some exemplary embodiments of the invention, pacing is applied at the AV node, to provide an artificial source of activation for the ventricle. Optionally, such pacing may be applied if the AV node sub-threshold signals were applied in a manner designed to allow an activation to arrive from the atria, but no such activation arrived. Pacing (e.g., applied 10-40 ms after such an expected arrival) is then optionally used to prevent bradycardia.

As can be appreciated, different disease states may result in different treatments. for example, the electrode locate and/or other signal application parameters may depends on the disease and/or on the particular detected arrhythmia and/or may vary based on eth desired change in AV activity. For example, in one patient small changes may be provided by stimulation of AV extensions and large changes by direct AV stimulation. In another patient, AT may be treated using AV stimulation, while AV reentrant arrhythmia treated by AV extension stimulation.

According to some embodiments, determining a subset of electrodes able to deliver sub-threshold electric bursts such that the bursts induce ventricular rate within the pre-determined range comprises:

inducing delivery of sub-threshold electric bursts to the subject's AV node through at least one of the plurality of electrodes;
determining whether the subject's ventricular rate is within the pre-determined range following the induction; and
if the ventricular rate is not within the pre-determined range following the induction, sequentially repeating said inducing and determining, each induction using a different electrode or combination thereof until the ventricular rate of said subject is within said pre-determined range.

According to some embodiments, in order to determine which electrode or electrodes are able to deliver sub-threshold electric bursts to the AV node in accordance with the present disclosure, the system's processor is configured to: (1) induce delivery of sub-threshold bursts through an electrode or a combination of electrodes of the plurality of electrodes; (2) measure after a certain time period, using input received from at least one heart-activity sensor, whether the bursts resulted in a ventricular rate which is within the pre-determined range, such as, but not limited to, 60-100 beats per minute; (3) if the ventricular rate following the induction in clause (1) is not within the pre-determined threshold, induce delivery of sub-threshold bursts through another electrode or another combination of electrodes and repeat measurement as in clause (2); (4) if the ventricular rate following the induction in clause (3) is within the pre-determined range, such as, but not limited to, 60-100 beats per minute, determine that this electrode or combination of electrodes is able to deliver sub-threshold electric bursts to the AV node of a subject.

According to some embodiments, the certain time period to measure whether the ventricular rate is within the pre-determined threshold is at least 30 seconds, preferably at least 1 minute. Each possibility represents a separate embodiment of the present invention.

In some exemplary embodiments of the invention, treatment continues for between 1 and 100 seconds, 1-100 minutes, 1-100 hours and/or intermediate or greater periods. Optionally, treatment is stopped, for example, after 20 seconds, 20 minutes and/or at other (smaller, intermediate or larger) times to detect if a disease state has passed. In chronic patients, the device may be programmed not to stop. Optionally or alternatively, stopping may be a function of detecting significant periods of time (e.g., between 1 and 20 minutes) where atrial arrhythmia is absent.

As can be appreciated, in some patients, treatment may continue (possibly on and off) for several years. In some patients, arrhythmia may be triggered by certain events (e.g., resting heart rate, cardiac demand) and treatment may be limited to such times, e.g., based on detection of such events. As noted herein, treatment may be preventive in nature (e.g., based on a prediction of a potential arrhythmia and/or may be responsive to a detected arrhythmia and/or ventricular suboptimality, for example using a known arrhythmia detection or prediction method).

### Exemplary System Components

According to some embodiments, the disclosed system further comprises a storage element, configured to store input sensed by the at least one heart-activity sensor. According to some embodiments, the storage element is connected to the processor and is configured to store electric field application protocols and/or clinical state predetermined parameters. In some embodiments, the storage element further comprises a controller, configured to control the process of reading information from the storage element. In some embodiments, the controller is configured to control writing processes to the storage element. In some embodiments, the controller is configured to control both reading and writing processes.

According to some embodiments, the storage element is configured to store information regarding the timing and characteristics of sub-threshold electric bursts provided to the subject. According to some embodiments, the system further comprises a wireless communication capability. According to some embodiments, the system further comprises a wireless communication capability able to transmit information sensed by the at least one heart activity sensor and/or stored in the storage component to a computer situated outside of the subject's body. Each possibility represents a separate embodiment of the present invention. According to some embodiments, the wireless communication capability is configured to transmit information regarding the timing and characteristics of sub-threshold electric bursts provided to the subject to a computer situated outside of the subject's body.

According to some embodiments, the system's processor is further configured to receive input from a computer situated outside of the subject's body. According to some embodiments, the information received from such an outside computer may be used to manually calibrate parameters of the sub-threshold electric bursts such as, but not limited to, frequency, duration, current and voltage. According to some embodiments, manually calibrating parameters of sub-threshold electric bursts enables to better calibrate the desired ventricular contraction rate.

According to some embodiments, system comprises a receiver element, connected to the processor and configured to receive information from an external computer situated outside of the subject's body. In some embodiments, received information is used to reprogram pre-determined parameters and/or electric field application protocols stored in the storage element. In some embodiments received information include safety parameters and/or calibration parameters and/or system operation parameters.

According to some embodiments, system further comprises a transmitter element, connected to the processor and configured to transmit information to an external computer and/or an external storage element situated outside of the subject's body. In some embodiments, transmitted information includes system operation reports and/or system log files.

According to some embodiments, at least part of the various elements of the disclosed system are encased in a single casing and/or are integrally formed. Each possibility represents a separate embodiment of the present invention. According to some embodiments, the at least one heart-activity sensor, the at least one electric-pulse generator, the plurality of electrodes and the processor of the present invention are encased in a single casing and/or are integrally formed. Each possibility represents a separate embodiment of the present invention. According to some embodiments, the disclosed system is configured to be introduced into the subject's heart as a single unit. According to some embodiments, the elements of the disclosed system are encased in a single casing and/or are integrally formed essentially in the shape of a stent configured to be inserted into the coronary sinus. Each possibility represents a separate embodiment of the present invention.

According to some embodiments, at least part of the elements of the disclosed system are encased in a single casing and/or are integrally formed. In some embodiments, casing further comprises anchoring element configured to anchor the casing at least partially within a blood vessel, for example the coronary sinus. In some embodiments, casing comprises a bio-inert material, for example titanium, gold, silver, platinum, and any combination hereof. In some embodiments, casing is in the form of a support element configured to be placed at least partly within a blood vessel and by a conformation change to anchor the element at least partly within a blood vessel, for example the coronary sinus.

According to another aspect, the present invention provides an implantable electrical stimulation system, wherein said system is configured to provide atrial fibrillation therapy to a subject, wherein the system comprises:

at least one electrical-pulse generator configured to generate sub-threshold electric bursts;
at least one heart-activity sensor;
a stent configured to be inserted into the coronary sinus, wherein said stent comprises at least one electrode, wherein said at least one electrode is functionally connected to said electrical pulse generator and configured to deliver said sub-threshold electric bursts to the subject's atrioventricular (AV) node; and
a processor configured to:
receive input from said at least one heart-activity sensor;
measure the ventricular rate of said subject using at least part of said input; determine whether said subject is undergoing atrial fibrillation based on at least part of said input; and
induce delivery of sub-threshold electric bursts to the subject's AV node through said at least one electrode if said subject is undergoing atrial fibrillation and has a ventricular rate above a pre-determined range.

According to some embodiments, the at least one electrode is a plurality of electrodes. According to some embodiments, each electrode of the plurality of electrodes is functionally connected to at least one electric-pulse generator. According to some embodiments, the processor is configured to select a subset of one or more of the plurality of electrodes able to deliver sub-threshold electric bursts to the subject's AV node. According to some embodiments, electrodes able to deliver sub-threshold electric bursts to the subject's AV node are electrodes able to deliver the bursts such that they induce a ventricular rate within a pre-determined range.

According to some embodiments, the processor, the at least one electric-pulse generator and the at least one heart-activity sensor are at least partly attached to and/or integrally formed with the stent. Each possibility represents a separate embodiment of the present invention. According to some embodiments, all elements of the disclosed system are at least partly attached to one another and/or integrally formed with one another. Each possibility represents a separate embodiment of the present invention.

According to some embodiments, all elements of the disclosed system are integrated with the stent, such that the entire system is configured to be inserted to the coronary sinus of the subject. According to some embodiments, all elements of the disclosed system are essentially comprised within a single casing. Without wishing to be bound by any theory or mechanism, a system having elements which are attached, integrally formed or encased in a single casing enables to easily insert the system into the subject's coronary sinus without running leads and/or metal wires inside the subject's body. According to some embodiments, the disclosed system comprising a stent does not comprise leads connecting between the elements, such that that the various elements are either electronically coupled or wirelessly connected. Each possibility represents a separate embodiment of the present invention.

According to some embodiments, the present invention provides an implantable electrical stimulation system, wherein said system is configured to provide atrial fibrillation therapy to a subject, wherein the system comprises:

at least one electrical-pulse generator configured to generate sub-threshold electric bursts;
at least one heart-activity sensor;
a stent configured to be inserted into the coronary sinus, wherein said stent comprises a plurality of electrodes, wherein said electrodes are functionally connected to said at least one electrical pulse generator and configured to deliver said sub-threshold electric bursts to the subject's atrioventricular (AV) node; and
a processor configured to:
receive input from said at least one heart-activity sensor;
measure the ventricular rate of said subject using at least part of said input; determine whether said subject is undergoing atrial fibrillation based on at least part of said input;
select a subset of one or more of said plurality of electrodes which are able to deliver sub-threshold electric bursts to the subject's AV node; and
induce delivery of sub-threshold electric bursts through said subset of one or more electrodes to the subject's AV node if said subject is undergoing atrial fibrillation and has a ventricular rate above a pre-determined range.

According to some embodiments, the present invention provides an implantable electrical stimulation system, wherein said system is configured to provide atrial fibrillation therapy to a subject, wherein the system comprises a stent configured to be inserted into the coronary sinus of said subject, said stent comprising:

at least one electrical-pulse generator configured to generate sub-threshold electric bursts;
a plurality of electrodes, wherein said electrodes are functionally connected to said at least one electrical pulse generator and configured to deliver said sub-threshold electric bursts to the subject's atrioventricular (AV) node; and
a processor configured to:
receive input from said at least one heart-activity sensor;
measure the ventricular rate of said subject using at least part of said input; determine whether said subject is undergoing atrial fibrillation based on at least part of said input;
select a subset of one or more of said plurality of electrodes which are able to deliver sub-threshold electric bursts to the subject's AV node; and
induce delivery of sub-threshold electric bursts through said subset of one or more electrodes to the subject's AV node if said subject is undergoing atrial fibrillation and has a ventricular rate above a pre-determined range.

According to some embodiments, the system comprises a stent configured to function as an electrode or a plurality of electrodes. Each possibility represents a separate embodiment of the present invention. According to some embodiments, the system comprises a stent configured to function as a distal end of an electrode or distal ends of a plurality of electrodes. Each possibility represents a separate embodiment of the present invention. According to some embodiments, the stent comprises at least one electrode. According to some embodiments, the stent comprises the distal end of at least one electrode. According to some embodiments, the stent comprises a plurality of electrodes.

According to some embodiments, the stent comprises the distal end of a plurality of electrodes.

According to some embodiments, the stent is configured to function as a distal end of at least one electrode and/or comprises the distal end of at least one electrode, wherein the at least one electrode is functionally connected to the electrical pulse generator and configured to deliver sub-threshold electric bursts to the subject's atrioventricular (AV) node. Each possibility represents a separate embodiment of the present invention.

According to some embodiments, the stent is configured to function as a distal end of a plurality of electrodes and/or comprises the distal end of a plurality of electrodes, wherein the plurality of electrodes are functionally connected to at least one electrical pulse generator and configured to deliver sub-threshold electric bursts to the subject's atrioventricular (AV) node. Each possibility represents a separate embodiment of the present invention.

According to some embodiments, the stent is configured to be positioned in the coronary sinus of the subject's heart, preferably in the opening of the coronary sinus into the right atrium, also known as the ostium of the coronary sinus or the coronary sinus orifice. Without wishing to be necessarily bound by any theory or mechanism, inserting the stent into the coronary sinus, which is located proximal to the AV node, enables electrodes comprised in the stent to deliver sub-threshold electrical bursts to the AV node, thus adjusting the ventricular rate to be within a pre-determined threshold. According to some embodiments, insertion of the stent into the coronary sinus enables stable fixation of the at least one electrodes, or at least their distal ends, proximally to the AV node. According to some embodiments, the entire disclosed system is configured to be positioned in the coronary sinus of the subject's heart, preferably in the opening of the coronary sinus into the right atrium, also known as the ostium of the coronary sinus or the coronary sinus orifice.

According to some embodiments, the stent may be composed of any known material known in the art to be suitable for producing stents, such as, but not limited to, nitinol. According to some embodiments, the stent is at least partly composed of a shape memory alloy, such as, but not limited to nitinol. According to some embodiments, at least a part of the stent is composed of an electrically conducting material. According to some embodiments, the system further comprises means of inserting the stent into the coronary sinus of the subject, such as, but not limited to a catheter-based insertion device.

According to some embodiments, the heart-activity sensor is selected from the group consisting of: atrial sensor configured to sense atrial activity, ventricular sensor configured to sense ventricular activity and a combination thereof. Each possibility represents a separate embodiment of the present invention.

According to some embodiments, the system's processor is further configured to: measure the ventricular rate of the subject using input received from the at least one heart-activity sensor; determine which of the at least one electrodes or combination thereof is able to deliver sub-threshold electric bursts to the subject's AV node such that the bursts induce a ventricular rate within a pre-determined range; and actuate delivery of sub-threshold electric bursts through said electrodes.

According to some embodiments, the present disclosure provides the disclosed system for the treatment of atrial fibrillation. According to other embodiments, the disclosed system is provided for the treatment of heart arrhythmias such as, but not limited to, atrial fibrillation, that may benefit from reduction of the effective refractory period in the AV node.

According to another aspect, the present disclosure provides a method of treating atrial fibrillation in a subject, the method comprising:

positioning a plurality of electrodes at or proximal to the subject's atrioventricular (AV) node, said electrodes configured to deliver sub-threshold electric bursts to the subject's AV node;
determining a subset of said plurality of electrodes or combination thereof are correctly positioned electrodes able to deliver sub-threshold electric bursts to the subject's AV node such that said bursts induce a ventricular rate within a pre-determined range; and
inducing delivery of sub-threshold electric bursts to the AV node through said correctly positioned subset of electrodes.

According to some embodiments, positioning a plurality of electrodes at or proximal to the subject's atrioventricular (AV) node relates to positioning at least the distal part of the electrodes at or proximal to the subject's AV node. According to some embodiments, positioning a plurality of electrodes at or proximal to the AV node relates to positioning at least part of the electrodes within or proximal to the coronary sinus, preferably the coronary sinus orifice. Each possibility represents a separate embodiment of the present invention. According to some embodiments, positioning a plurality of electrodes at or proximal to the subject's atrioventricular (AV) node comprises positioning additional elements required for the electrodes' ability to deliver sub-threshold electric bursts, such as, but not limited to, at least one electric-pulse generator. According to some embodiments, positioning a plurality of electrodes comprises positioning a stent comprising said plurality of electrodes within the coronary sinus of the subject. According to some embodiments, the method comprises positioning a stent within the coronary sinus of the subject, the stent comprising the plurality of electrodes.

According to some embodiments, the phrase “correctly positioned electrodes” refers to electrodes which are positioned such that they are close enough to and/or aligned with the AV node and are thus able to deliver sub-threshold electric bursts to the AV node of the subject. Of note, all of the electrodes in the plurality of electrodes are configured to be able to deliver sub-threshold electric bursts to the AV node, but only correctly positioned electrodes are able to do so. Without wishing to be necessarily bound by any theory or mechanism, using the disclosed method obviates the need to precisely position a single electrode exactly at the AV node thus enabling easy insertion and use of the electrodes. According to some embodiments, the disclosed method is performed at least partly using the disclosed system.

According to some embodiments, the method further comprises sensing heart activity using at least one heart-activity sensor. According to some embodiments, the method further comprises using input received from the at least one heart-activity sensor to determine when to induce delivery of the sub-threshold bursts and when to end the delivery of the sub-threshold bursts.

According to some embodiments, the method further comprises determining whether the subject is undergoing atrial fibrillation based on at least part of the input received from the at least one heart-activity sensor. According to some embodiments, inducing delivery of sub-threshold electric bursts to the AV node through the correctly positioned electrodes is inducing delivery only if the subject is undergoing atrial fibrillation and/or has a ventricular rate above a pre-determined range. Each possibility represents a separate embodiment of the present invention. According to some embodiments, inducing delivery of sub-threshold electric bursts to the AV node through the correctly positioned electrodes is inducing delivery only if the subject is undergoing atrial fibrillation and has a ventricular rate above a pre-determined range. According to some embodiments, having a ventricular rate above a pre-determined range is having the ventricular rate for more than 1 minute. According to some embodiments, a ventricular rate above a pre-determined range is a ventricular rate of above 100 Beats Per Minute.

According to some embodiments, the method further comprises arresting delivery of the sub-threshold electric bursts to the AV node through the correctly positioned electrodes. According to some embodiments, arresting delivery is only if the subject is no longer undergoing atrial fibrillation and/or has a ventricular rate within or below the pre-determined range. Each possibility represents a separate embodiment of the present invention. According to some embodiments, arresting delivery is only if the subject is no longer undergoing atrial fibrillation and has a ventricular rate within or below the pre-determined range.

According to some embodiments, the method further comprises determining whether the subject undergoes atrial fibrillation, wherein delivery of the sub-threshold electric bursts is induced only if the subject is determined to undergo atrial fibrillation.

According to some embodiments, determining which of said plurality of electrodes or combination thereof are correctly positioned electrodes comprises:

inducing delivery of sub-threshold electric bursts to the subject's AV node through at least one of said electrodes;
determining whether the subject's ventricular rate is within a pre-determined range following the induction; and
if the ventricular rate is not within said pre-determined range following the induction, sequentially repeating said inducing and determining, each induction using a different electrode or combination thereof until the ventricular rate of said subject is within said pre-determined range; wherein electrodes able to deliver sub-threshold electric bursts to the subject's AV node such that the ventricular rate of said subject is within said pre-determined range are correctly positioned electrodes.

According to some embodiments, electrodes able to deliver sub-threshold electric bursts to the subject's AV node such that the ventricular rate after said delivery is within the pre-determined range are correctly positioned electrodes.

According to some embodiments, a ventricular rate within the pre-determined range is a ventricular rate which is within the pre-determined range for at least a consecutive minute, preferably consecutive 2, 3, 4 or 5 minutes. Each possibility represents a separate embodiment of the present invention.

According to some embodiments, the plurality of electrodes or the distal ends of the plurality of electrodes is comprised in a stent. Each possibility represents a separate embodiment of the present invention. According to some embodiments, the method further comprises positioning the stent within the coronary sinus, preferably at or within the orifice of the coronary sinus.

According to some embodiments, the present disclosure provides a method for treating atrial fibrillation in a subject, the method comprising:

positioning a stent within the coronary sinus, preferably within the orifice of the coronary sinus, wherein the stent is configured to function as at least one electrode and/or comprises at least one electrode and wherein said at least one electrode is configured to deliver sub-threshold electric bursts to the subject's atrioventricular (AV) node; and
inducing delivery of sub-threshold electric bursts to the subject's AV node through said at least one electrode. Each possibility represents a separate embodiment of the present invention.

According to some embodiments, the stent is configured to serve as the distal end of the at least one electrode.

According to some embodiments, the present invention provides a method of treating atrial fibrillation in a subject, the method comprising:

selecting a correctly positioned subset of one or more electrodes of a plurality of electrodes comprised in a stent positioned within the subject's coronary sinus, wherein said subset is able to deliver sub-threshold electric bursts to the AV node of said subject such that said bursts induce a ventricular rate within a pre-determined range; and
inducing delivery of sub-threshold electric bursts to the AV node through said subset of one or more electrodes.

According to some embodiments, the method further comprises positioning the stent comprising a plurality of electrodes within the coronary sinus of the subject.

According to some embodiments, the method further comprises positioning a stent within the coronary sinus of the subject, the stent comprising: a plurality of electrodes; at least one electric-pulse generator, wherein each electrode of the plurality of electrodes is functionally connected to at least one electric-pulse generator; at least one heart-activity sensor; and a processor functionally connected to the plurality of electrodes and/or the at least one electric pulse generator. Each possibility represents a separate embodiment of the present invention.

According to some embodiments, delivery of sub-threshold electric bursts is induced only is the subject undergoes atrial fibrillation and has a ventricular rate above the pre-determined range, such as, but not limited to, about 100 BPM.

According to some embodiments, the sub-threshold electric bursts according to the disclosed method are configured to be delivered only when a subject is suffering from an episode of atrial fibrillation and not constantly.

Without wishing to be necessarily bound by theory or mechanism, the at least one battery which powers the disclosed system is able to last for a long period of time, such as at least 5, 10, 15 or 20 years, since the system is configured to only sent sub-threshold electric bursts when a subject is suffering from an episode of atrial fibrillation with rapid ventricular rate. Each possibility represents a separate embodiment of the present invention.

According to some embodiments, treating atrial fibrillation using the disclosed systems and methods results in slowing of ventricular rate. According to some embodiments, treating atrial fibrillation using the disclosed systems and methods results in slowing of ventricular rate to below a pre-determined range, such as, but not limited to, 100 BPM.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Effect of Stimulation

The present invention is optionally configured to apply an electric field to treat symptoms of atrial fibrillation and other related arrhythmias, for example atrial flutter.

Reference is now made to FIG. 1D, depicting electrocardiogram (ECG) recordings during atrial flutter 50, atrial fibrillation 51 and atrial tachycardia 60 arrhythmias, and following electric field application 53. Under arrhythmia conditions, higher contraction rate of the atrium, represented by atrial activity 58 wave may lead to higher contraction rate of the ventricle, represented by ventricle activity 55 wave (also termed QRS complex) as indicated by a short interval 54 which indicates the time between each contraction of the ventricle. Higher and/or irregular contraction rate of the ventricle may in inefficient blood refill of the ventricle, reduced cardiac output and/or overworking of the heart and/or other adverse effects on the heart of the body.

In both atrial flutter 50 and atrial tachycardia 60, ECG recordings indicate rapid atrial activity 58 (also termed p waves) followed by rapid ventricle activity 53 (which can be seen by the frequency of the QRS complex). In this example of atrial flutter 50 ECG, the ratio between atrial activity and ventricle activity is 2:1, meaning that for 2 contractions of the atria the ventricle contracts once. According to some embodiments, electric field application to the AV node, and/or AV node extensions may modify this ratio to a 4:1 ratio, representing a slower and possibly more desirable, ventricular rate. In the presented Atrial tachycardia ECG 60, ventricle contracts at a rate of 240 contractions per minute. According to some embodiments, electric field application to the AV node, and/or AV node extensions may reduce the frequency of atrial activity 58 and/or increase the ratio between atrial activity 58 and ventricle activity 55, leading to slower heart rate. Other exemplary ratios are 1:3, 1:5 and/or intermediate ratios. Optionally or alternatively, the target of treatment is an actual ventricular contraction rate (or range) and/or uniformity thereof.

During Atrial fibrillation 51, atrial activity 58 is chaotic and represents irregular electric activity of the atria. According to some embodiments, subthreshold electric bursts 56 can be applied to the AV node and/or extensions, during ventricle activity 55, for example, during every second ventricle activity 55 wave, as described in 51. In some embodiments, the applied bursts transiently lower the ability of AV node to deliver pulses to the ventricle. According to some embodiments, during burst application, AV node signal conductivity is lowered from a baseline conductivity level 62, to a desired conductivity level. In some embodiments, lowering AV node conductivity, creates a block in conductivity 57, as seen in the AV node conductivity 52 panel. As an alternative view suitable for some embodiments, it is during breaks in sub-threshold application, that a (higher) conductivity window is opened.

It is noted that the baseline shown is actually an average, as the actual conductivity depends on the state within the depolarization cycle of the tissue whose response is being modified.

In some embodiments, during the blocking of the conductivity 57, atrial signals are less likely to pass through AV node and into the ventricle. In some embodiments, reducing conductivity level 59 of the AV node, depends on the response of AV node tissue to the burst. In some embodiments, reducing conductivity level 59 of the AV node, depends on the target tissue of the burst (e.g., depolarization state, disease state). In some embodiments, opening (61) and closing (59) the conductivity window is gradual, and may depend, for example, on tissue and/or patient and/or cycle and/or signal parameter specific characteristics, and/or may vary between each burst.

According to some embodiments, the time in which conductivity window remains open, depends on the AV node tissue. In some embodiments, the time in which the conductivity window remains open, depends on burst parameters and or/timing parameters of the burst. In some embodiments, opening of conductivity window is determined by a delayed response of the target tissue to the subthreshold burst stimulation. According to some embodiments, the time in which the window remains open may vary between bursts and/or selected electrodes and/or target tissue. According to some embodiments, variations in opening and closing of the conductivity window may lead to variations in burst effect. According to some embodiments, subthreshold electric burst may have a different effect depending on when it's applied relative to atrial activity 58. According to some embodiments, electrode mapping may provide information regarding burst application timing and response of the tissue depending on, for example, timing and other parameters.

In some embodiments, providing only certain windows for signal propagation from atria to ventricle may lead to reduction in ventricle contraction rate, as indicated by a longer interval 54, between each ventricle contraction, as depicted in ECG recording panel 53. Longer intervals may allow a more efficient blood refill of the ventricle, which may lead to an increase in cardiac output.

In any case, it is noted that in many atrial arrhythmia atrial activity is difficult to predict or even measure (e.g., which signal might reach the AV node), thus, it is a statistical process which decides when and if an activation signal will pass during the window and to the ventricle. Optionally, if insufficient activation signals pass, the windows will be increased in size and/or other parameters changed. Similarly, windows may be closed and/or moved closer together if too many such signals pass.

Optionally or alternatively, the duration of a conductivity window may be selected so that a certain number of activations can pass. For example, if the window is narrower than a ventricle refractory period, only one signal could pass per window.

In some exemplary embodiments of the invention, the burst schedule is uniform and/or adapted as needed, for example as just described. Optionally or alternatively, a predefined sequence of burst lengths, delays and/or other parameters may be used, for example, a series of bursts designs to provide windows of gradually increasing and decreasing widths.

It is noted that in some embodiments, the sub-threshold bursts are used to define windows of time, during which activations may propagate to the ventricle and/or windows where they cannot. Optionally or alternatively, the sub-threshold bursts are used to generally increase and/or decrease AV node conductivity and/or delay, so as to generally increase or decrease the probability of an activation passing therethrough and/or thereby to a ventricle.

### Exemplary Implantable System

In order to affect signal conduction pathways between the atria to ventricles, the disclosed invention provides an implantable system which is configured to apply an electric field, in the form of sub-threshold and/or supra-threshold electric bursts.

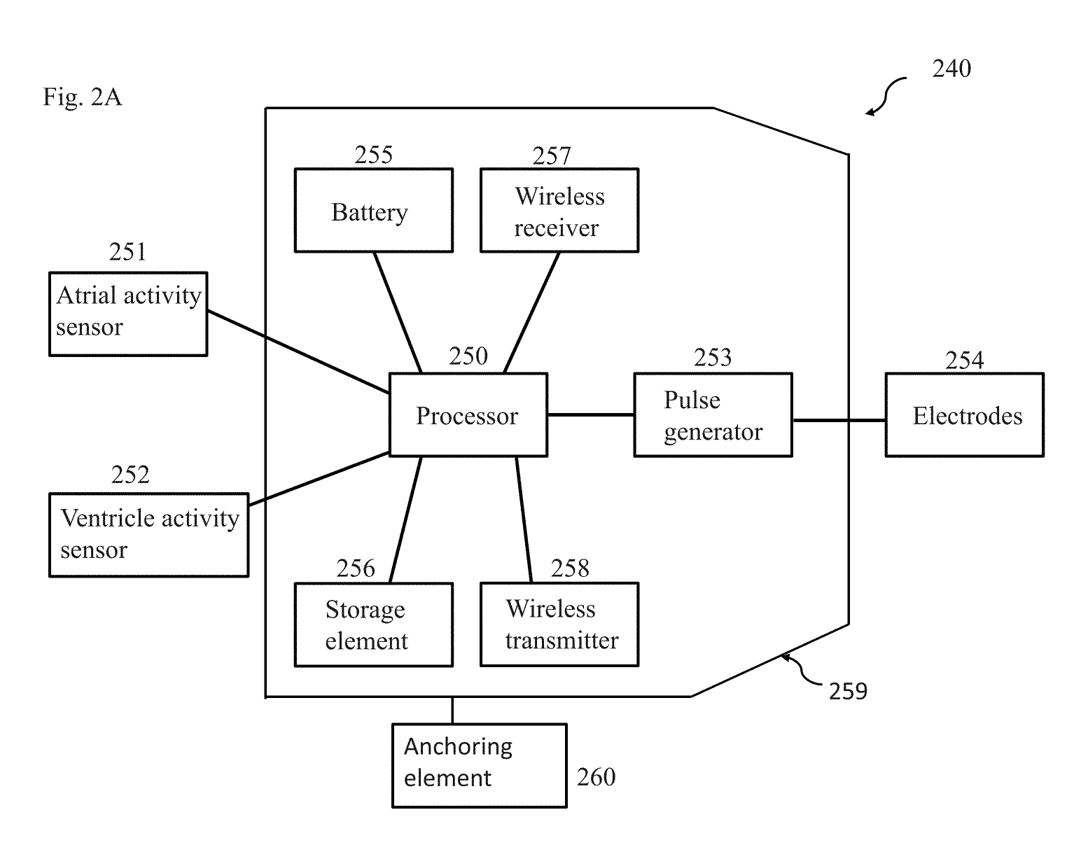

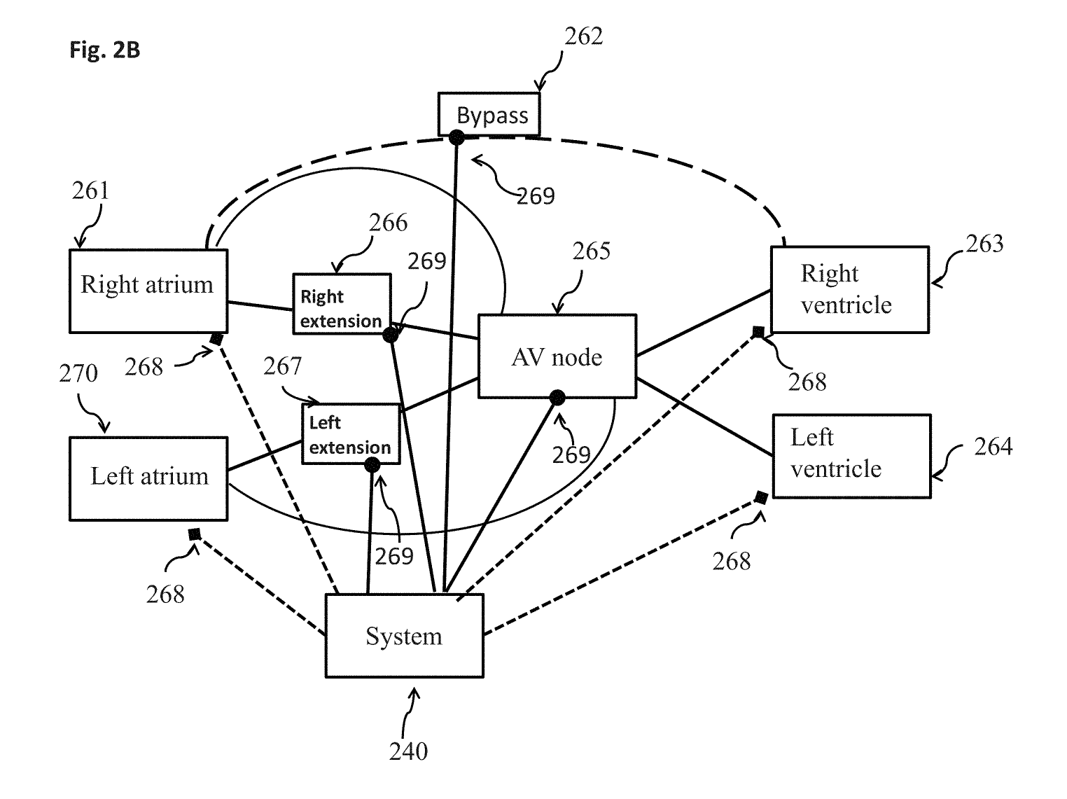

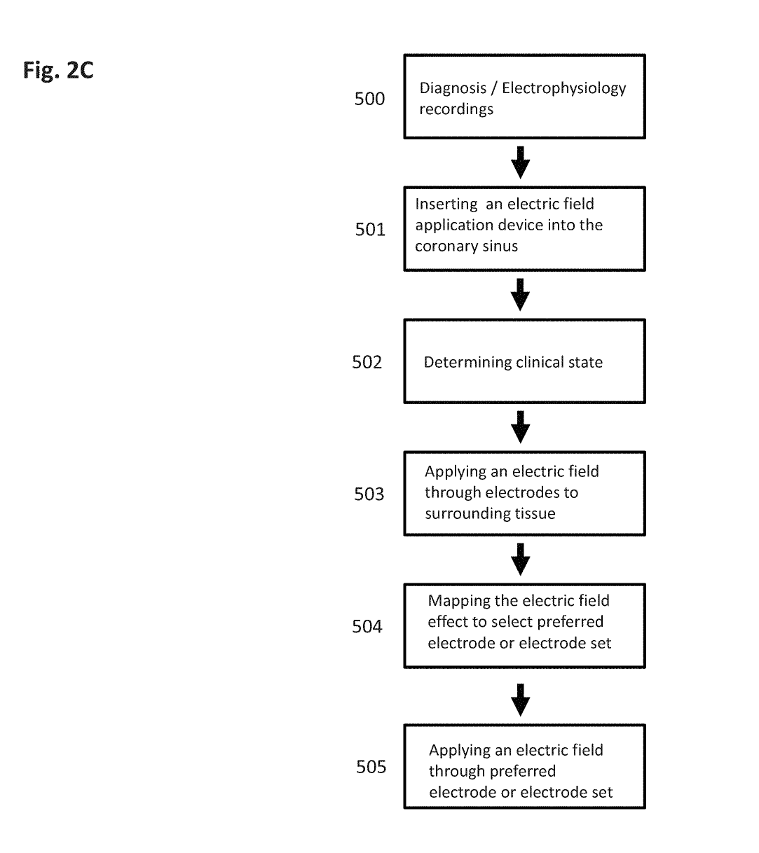

Reference is now made to FIG. 2A, depicting an implantable system 240 configured to apply an electric field to affect signal propagation between the atria and the ventricles of the heart, in accordance with some embodiments of the invention. System 240 comprising a processor 250, connected to pulse generator 253, optionally under control of processor 250, which pulse generator 253 is configured to generate sub-threshold and/or other electric bursts according to some embodiments. In some exemplary embodiments of the invention, electric bursts are delivered from the pulse generator 253 to one or more electrodes 254 positioned within the coronary sinus. In some exemplary embodiments of the invention, one or more of electrodes 254 are positioned outside the coronary sinus, for example, at locations proximal to signal conduction pathways. In some exemplary embodiments electrodes are positioned at locations proximal to AV node, and/or AV node input pathways, for example AV node extensions. Optionally, the electrodes are located within 0-5 mm from a CS ostium.

In some exemplary embodiments, system 240 further comprises a physiological sensor, for example, an atrial activity sensor 251 and/or a ventricle activity sensor 252 (e.g., electrical or other sensors adapted to be attached to a cardiac muscle wall). Signals delivered by a sensor (e.g., 251, 252) allow processor 250 to determine when and/or if to apply an electric field to the tissue (and/or parameters thereof). In some exemplary embodiments, signals delivered by sensors 251, 252 after an electric field was applied, allow processor 250 to determine whether the applied field was efficacious and/or whether the desired effect was reached. Optionally, the signal application and/or parameters will be changed or maintained. For example, a table of alternatives, for example stored in memory, may be used to provide a modified therapy based on results of a previous therapy. Optionally, heart activity sensors 251, 252 are configured to sense heart activity signals and to deliver the signals to processor 250. In some exemplary embodiments of the invention, atrial activity sensor 251 is configured to sense one or more of atrial contraction, atrial rate, atrial depolarization, atrial repolarization and/or a sub- or full combination thereof. In some exemplary embodiments of the invention, ventricle activity sensor 252 is configured to sense one or more of ventricle contraction, ventricle rate, ventricle depolarization, ventricle repolarization and/or a combination or sub-combination thereof. According to some embodiments, one or more stimulation electrodes are configured to act as sensors for atrial and/or ventricle activity.

Optionally, system 240 comprising a battery 255 connected to processor 250 and configured to supply power to system 240. Optionally, battery 255 is connected to at least one element of the system, such that the system does not require an additional external energy source.

In some embodiments, processor 250 is connected to a storage component 256. Optionally, component 256 (e.g., RAM or EEPROM memory) is configured to store one or more of heart activity log information, electric field application protocols, parameters and/or decision logic. In some embodiments, a wireless transmitter 258 is connected to processor 250, and optionally configured to transmit system activity related data and/or data stored in storage component 256 to an external computer and/or a mobile device. In some exemplary embodiments a wireless receiver 257 is connected to processor 250, and is configured to receive information from an external computer and/or a mobile device. An external computer or mobile device can be used, in some embodiments, to reprogram system 240 by delivering signals through wireless receiver 257 to processor 250.

According to some embodiments, at least some of the components described herein are encased in a single casing 259 and/or are integrally formed. In some exemplary embodiments, casing 259 further comprises an anchoring element 260 configured to anchor casing 259 at least partially within a blood vessel, for example the coronary sinus. In some embodiments, casing 259 comprises a bio-inert material, for example titanium, gold, silver, platinum, and any combination hereof. In some embodiments, casing 259 is in the form of a support element configured to be placed at least partly within a blood vessel and by a conformation change to anchor the element at least partly within a blood vessel, for example the coronary sinus.

Exemplary System Interaction with Tissue

According to some exemplary embodiments of the invention, an electric field is applied through electrodes placed at least partially within the coronary sinus and/or at locations in vicinity to signal conduction pathways, for example near the AV node and/or AV node extension.

Reference is now made to FIG. 2b, depicting system 240 interactions with heart tissue, according to some embodiments of the invention. In the heart, signals propagate from the right atrium 261 and the left atrium 270 to the right ventricle 263 and the left ventricle 264 through the AV node 265 region. Some of the signals arrive to the AV node 265 through the left inferior extension 267 and/or the right inferior extension 266 (where they exist), and some arrive directly from the atria. In the AV node 265 region the signals arriving from the atria are slowed down before they are delivered to the ventricles. However, under some conditions, signals are delivered to the ventricles without being slowed down by the AV node 265, or in some cases they pass through an alternative signal conduction pathway, e.g., a bypass 262 and not (or in parallel) through the AV node 265 region. The result in either case is inefficient contractions of the ventricles, which leads to reduced cardiac output.

According to some exemplary embodiments, system 240 comprising at least one processor, at least one electrode, and at least one heart activity sensor, configured to apply an electric field is implanted next to the heart and/or within the coronary sinus. According to some exemplary embodiments, one or more optional heart activity sensors 268 sense heart activity at the atria, ventricle(s), AV node, AV node extensions and/or other associated tissue and/or other cardiac locations and transmits the information to system 240. According to some exemplary embodiments, system 240 analyses heart activity information, and determines whether to apply an electric field through electrodes 269 to the heart. According to some exemplary embodiments electrodes can deliver an electric field to affect signals propagating through one or more of AV node 265 region, left AV node extension 267, right AV node extension 266, bypass 262 or any combination hereof. According to some exemplary embodiments, when the atrial signal propagates through a bypass 262 signal conduction pathway and not through the AV node 265 region, system 240 is configured to deliver an electric field to affect the signals propagating through bypass 262. According to some exemplary embodiments, system 240 applies an electric field as sub-threshold electric bursts, supra-threshold electric bursts or a combination hereof.

It is a particular feature of some embodiments of the invention that conduction through the AV node is affected by stimulation of associated tissue (e.g., AV node extensions), to affect the AV node (or other pathway). Optionally, associated tissue includes tissue that is in close proximity, including signal conduction pathways and/or tissue which is biologically set up to act as inputs to tissue in the pathway.

It is a particular feature of some embodiments of the invention, that tissue targeted to be directly affected by stimulation is muscle tissue, for example, muscle fiber cells (e.g., rather than or in addition to nervous tissue). Optionally, the effect on muscle fiber explains at least 20%, 50%, 80% or intermediate or greater percentages of the modification of conduction, for example, as measured as a percentage of signals blocked and/or as an effect on delay (% change in propagation time).

Exemplary Method for Electric Field Application

According to some embodiments of the invention, the system described herein is configured to be implanted in patients suffering from arrhythmia symptoms as diagnosed by an expert in the field. Arrhythmia diagnosis, system transplantation and/or system operation are optionally carried out as described herein.

Reference is now being made to FIG. 2C, which is a flowchart of a method of electrical field application, in accordance with some exemplary embodiments of the invention.

Optionally, Arrhythmia diagnosis is performed by recording patient heart activity using electrodes and/or other sensors placed outside the body, and/or by inserting electrodes of an electrophysiological mapping catheter at 500 into blood vessels or heart lumens, for example the coronary sinus or using other methods, such as imaging, for example, as known in the art. An expert in the field can determine the arrhythmia type based on the recordings. If the patient suffers from symptoms of atrial fibrillation, atrial flutter or related arrhythmias, the expert can suggest implanting the disclosed system near or within the patient's heart.

Optionally, once diagnosed, an electric field application system is inserted, at least partially into the patient's coronary sinus at 501. According to some exemplary embodiments, electrodes are configured to be placed at locations that were shown during diagnosis to be in vicinity to signal conduction pathways, for example AV node and/or AV node extensions. Optionally, a same electrode as used for diagnosis is used for treating.

According to some exemplary embodiments, the implanted system comprises one or more heart activity sensors, for example configured to be placed in different locations and/or within or close to the coronary sinus, for example atria, and/or ventricles, and/or AV node region. According to some embodiments, at least one electrode is configured to sense atrial activity and/or ventricle activity. Optionally, a heart activity sensors is further configured to sense one or more heart activity signals selected from the list of atrial activity, ventricle activity, AV node activity and/or a combination or sub-combination of the listed activity signals. In some embodiments, system's processor receives heart activity signals, process and analyzes them to evaluate the current clinical state of the patient, at 502. Clinical state is evaluated by comparing sensed heart activity signals to pre-determined parameters. Optionally, analysis uses an external processor.

According to some exemplary embodiments, if processor determines to apply an electric field, then an application protocol is selected from a list of application protocols stored in a storage component (possibly a single protocol, optionally with multiple alternative parameter setting possibilities), connected to the processor.

An electric field is applied at 503 optionally using an electrode or electrode set placed within the coronary sinus and/or near signal conduction pathways, for example the AV node or AV node extensions. In some embodiments the electric field is applied as a sub-threshold or a supra-threshold electric burst. In some embodiments, electric field is applied through at least one of the electrodes which is placed near the coronary sinus ostium. According to some exemplary embodiments, electric field is applied directly to muscle tissues at the vicinity of the AV node. Optionally, electric field is applied to AV node input pathways, for example AV node extensions. In some embodiments, electric field is applied directly to neural tissue and/or a cardiac fatpad. In some embodiments an electrode mapping process follows and/or precedes the electric field application process.

According to some exemplary embodiments, the effect of the applied electric field is analyzed by receiving heart activity signals from heart activity sensors and determining whether the applied electric field has resulted with the desired effect. If the desired effect is reached, the electrode or electrode set which delivered the electric field is indicated as a selected electrode or electrode set at 504. According to some exemplary embodiments, the system's processor is configured to determine whether to apply an electric field through the selected electrode or electrode set, or to continue and map the resulted effect when applying an electric field through other electrodes. According to some exemplary embodiments, if the desired effect was not reached then an electric field is applied through another electrode or electrode set and/or using different parameter set values.

According to some exemplary embodiments, a second electric field is applied through a selected electrode or electrode set to the surrounding tissue at 505, optionally followed by a new clinical state evaluation.

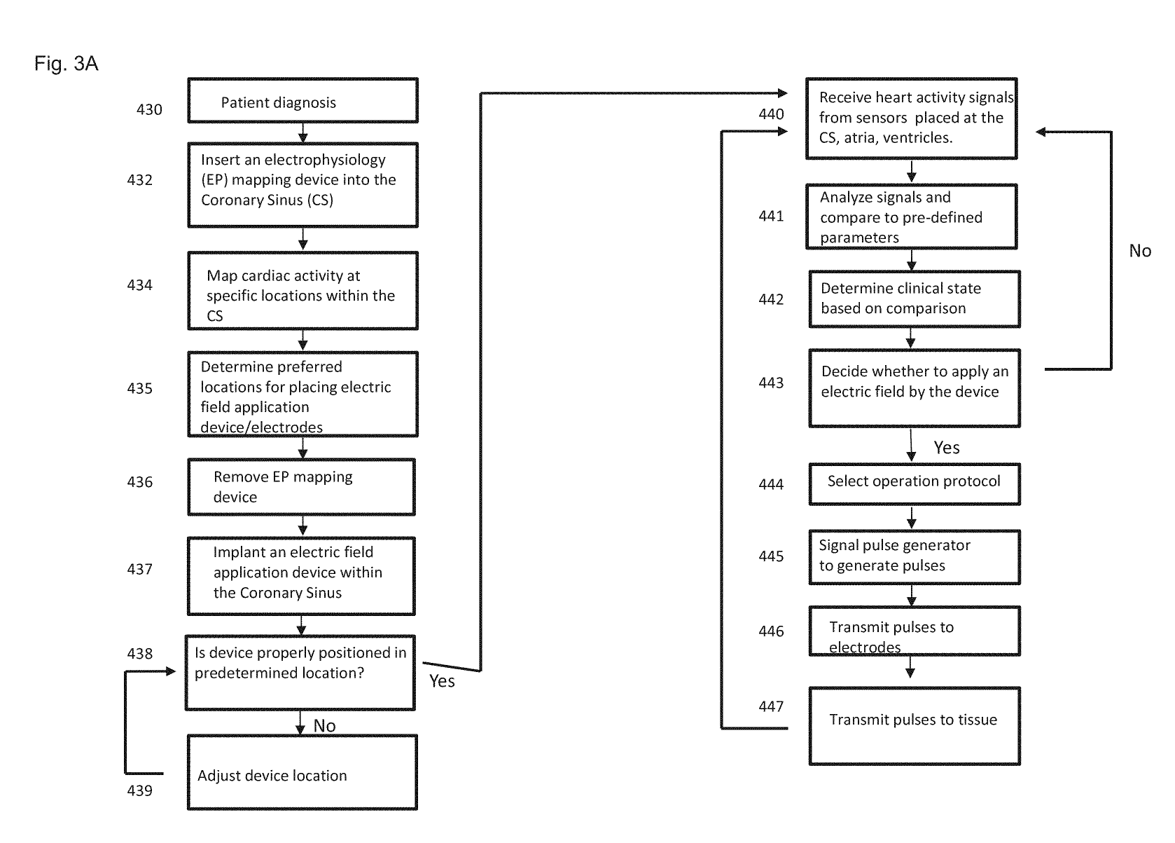

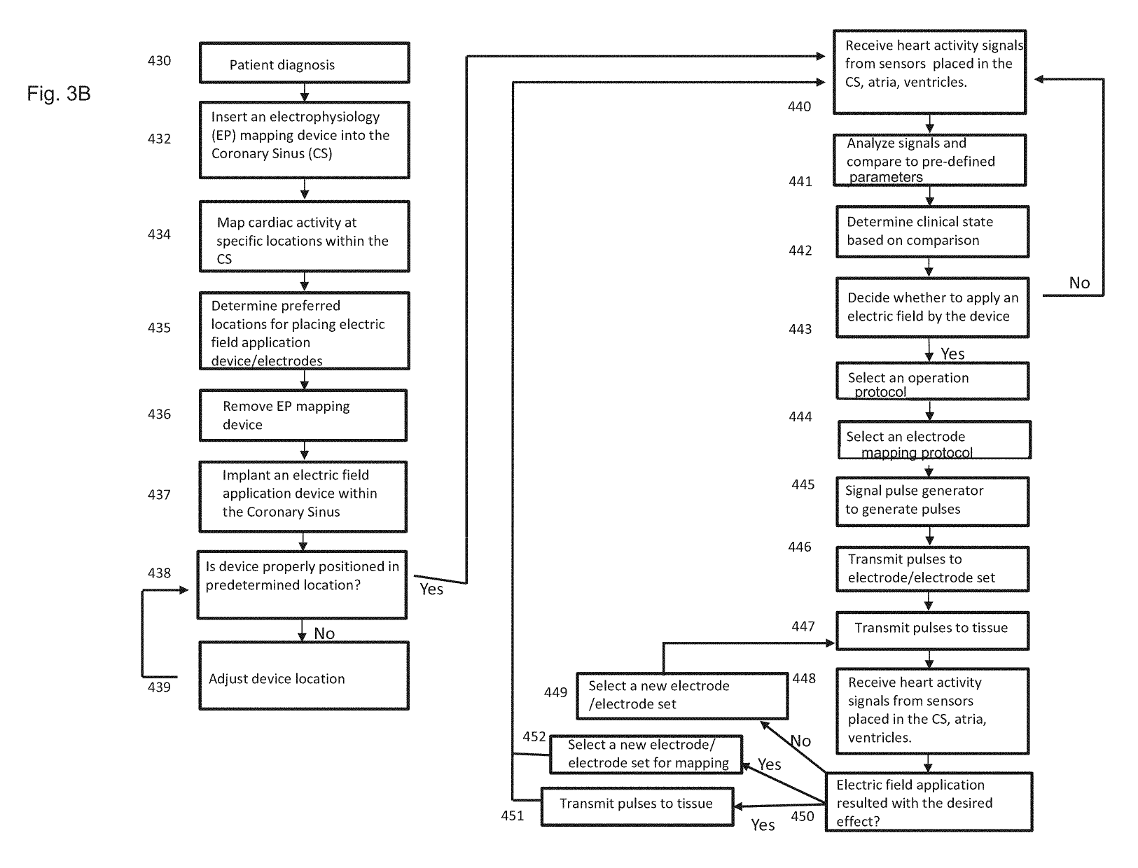

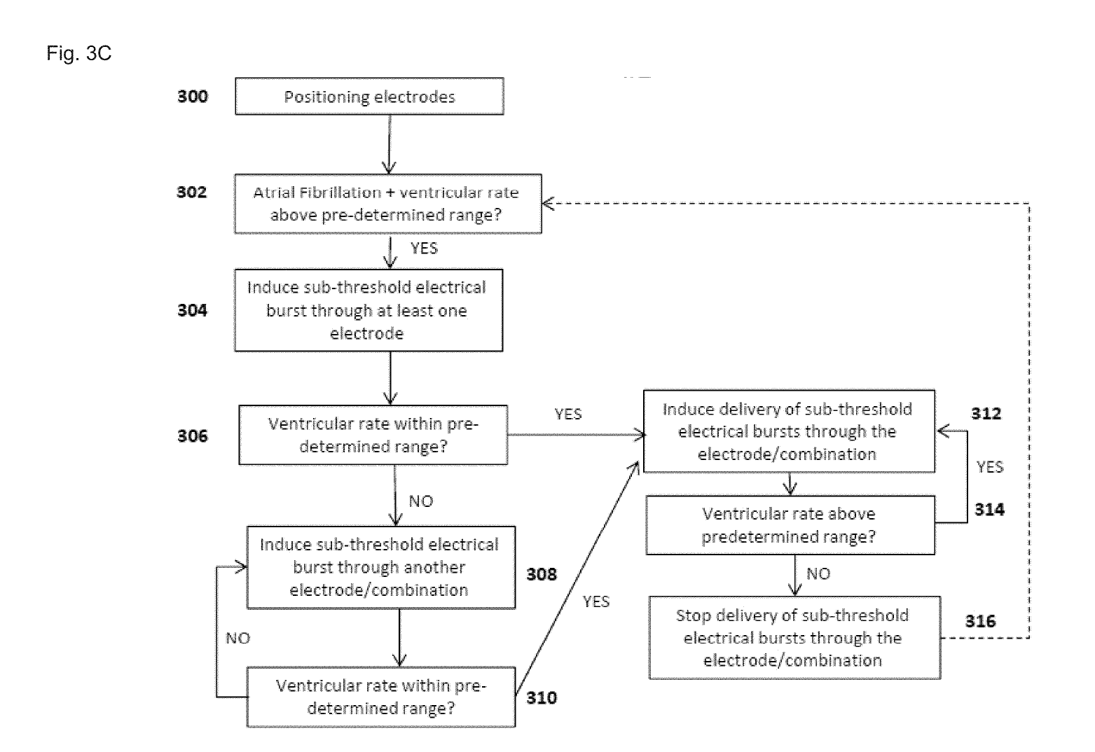

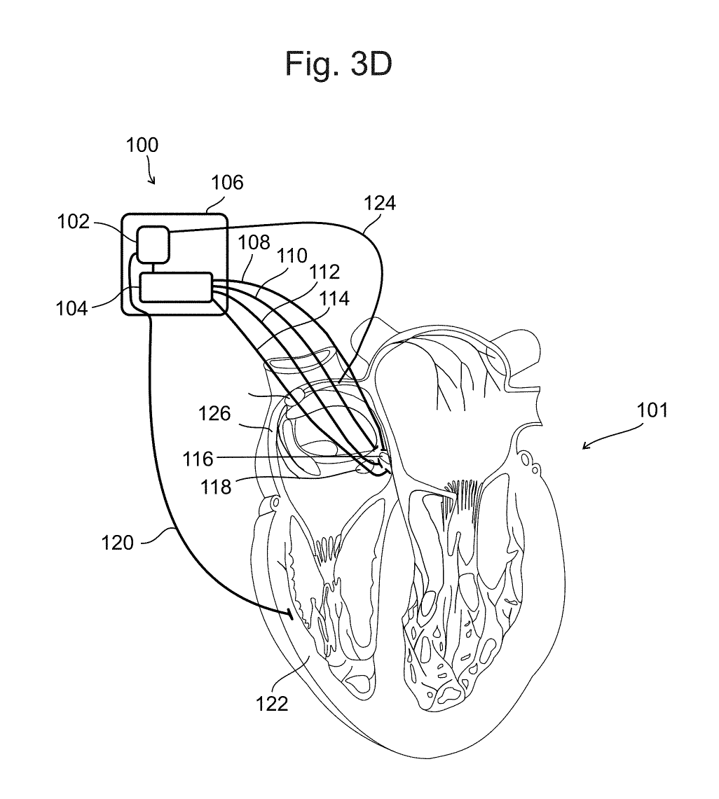

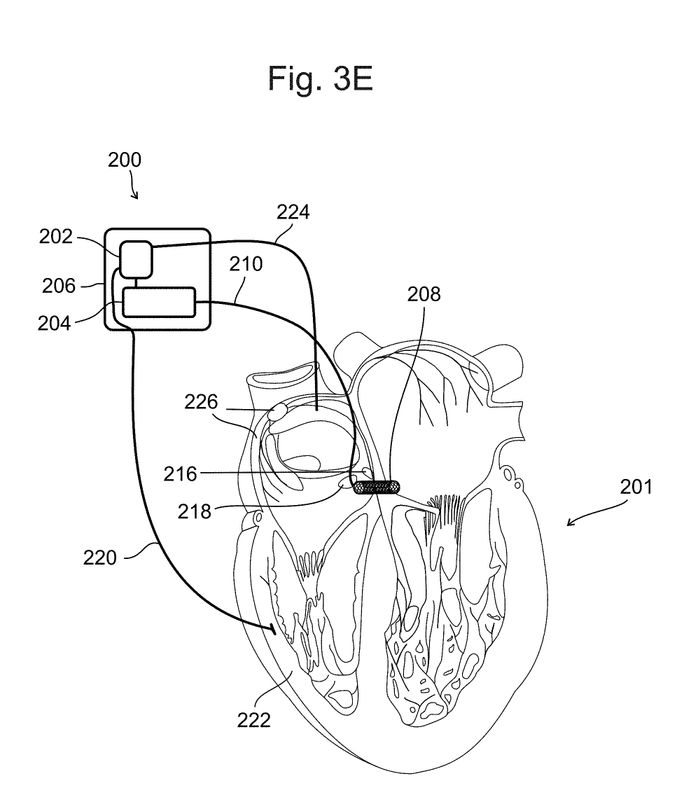

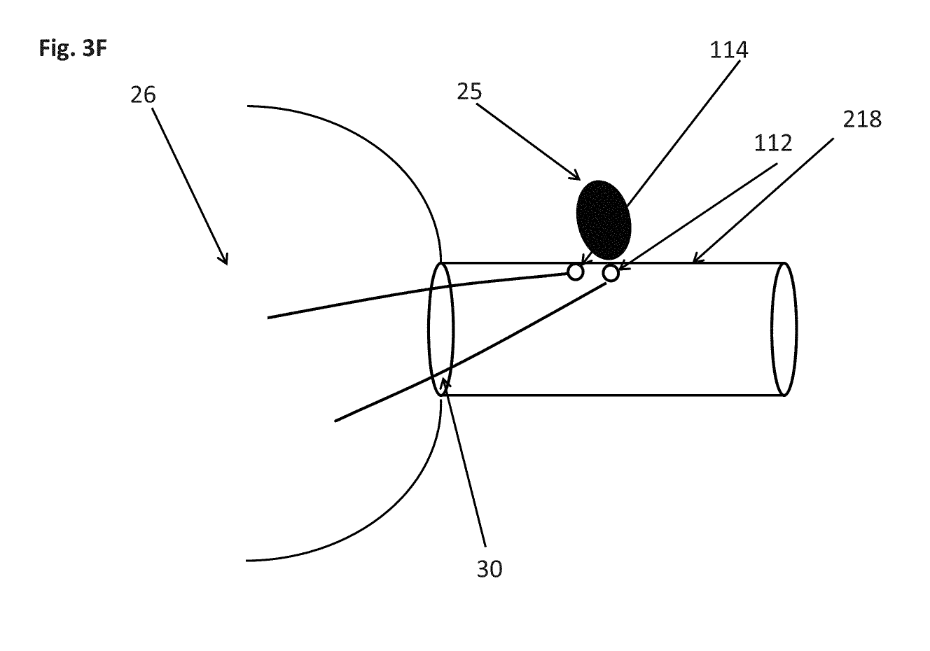

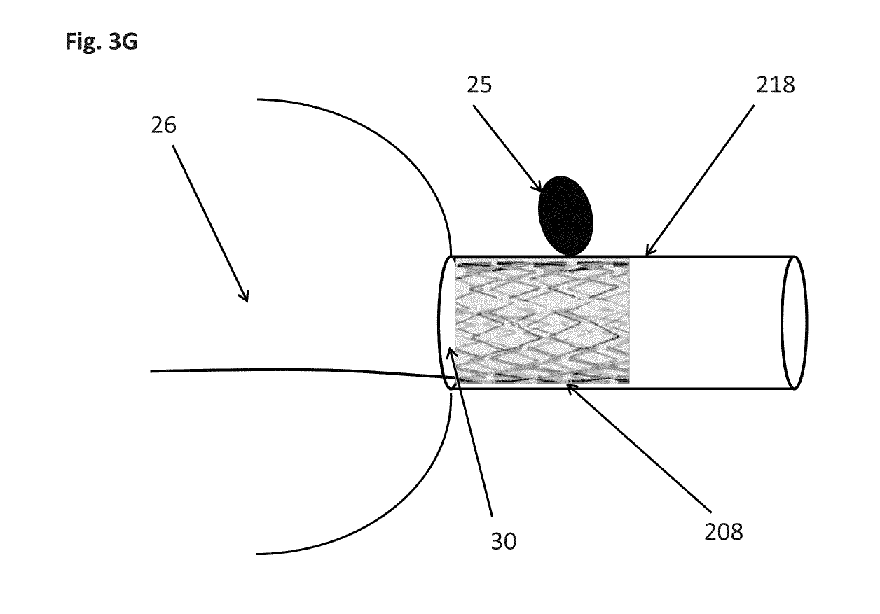

Reference is now made to FIG. 3A, depicting an overview of an electric field application procedure according to some embodiments of the present invention, possibly in greater detail. A patient suffering from arrhythmia symptoms is diagnosed by an expert in the field for example a physician, at 430. For example, arrhythmia can be characterized as an abnormal heart rhythm, and can be divided into 4 main types: extra beats, supraventricular tachycardias, ventricular arrhythmias, and bradyarrhythmias. Extra beats include premature atrial contractions and premature ventricular contractions. Supraventricular tachycardias include atrial fibrillation, atrial flutter, and paroxysmal supraventricular tachycardia. Ventricular arrhythmias include ventricular fibrillation and ventricular tachycardia.

Arrhythmia diagnosis can be performed by recording patient's heart activity using electrodes and/or other sensors placed outside the body, and/or by inserting electrodes of an electrophysiological mapping catheter at 432 into blood vessels or heart lumens, for example the coronary sinus.

According to some exemplary embodiments, electrodes inserted into the patient's heart lumens or blood vessels, for example the coronary sinus, are configured to record electrophysiological activity parameters to map cardiac activity at 434. Electrophysiological activity parameters include for example, atrial contraction rate and/or ventricle contraction rate at specific locations within the blood vessel.

According to some exemplary embodiments, electrophysiological mapping at 434 is followed by determination of preferred locations within the coronary sinus for placing the electric field application system and/or electrodes at 435. According to some embodiments, preferred locations are specific locations that are found in close proximity to signal conduction pathways involved in the diagnosed arrhythmia, for example the AV node and/or AV node extensions. According to some embodiments, preferred locations are specific locations that are found near the coronary sinus ostium.

According to some exemplary embodiments, the electrophysiological mapping catheter is removed at 436 and an electric field application system is implanted within or partly within the coronary sinus at 437. Alternatively, the mapping catheter is used as a permanent lead. Optionally, a same controller is used for mapping and therapy. Alternatively, after mapping an external controller is replaced by an implantable controller. In some exemplary embodiments of the invention, mapping may also be performed after implantation, for example, an initial mapping (e.g., during implantation or device setup after implantation) or after a time, for example, a month or more, for example, in response to reduced efficacy of treatment.

According to some exemplary embodiments, a validation process is performed at 438, to assure that system's electrode or electrode set is placed in preferred locations within the coronary sinus as previously determined. Optionally, validation uses impedance measurement to check electrode contact quality and/or uses test stimulation to determine if an expected effect is detected. Optionally, an atria is artificially paced abnormally (or various pharmaceuticals provided to induce certain cardiac states) to ensure that correct (e.g., arrhythmia) conditions for testing the functionality of the system, are provided.

According to some exemplary embodiments, if system's electrode or electrode set is placed in a location which is not a preferred location then adjustments are performed at 439. In some embodiments, adjustments include rotating and/or moving electrodes or system inside the coronary sinus. In some exemplary embodiments of the invention, however, adjustment comprises changing an electrode to be used and/or pulse sequence, rather than moving the electrodes and/or anchoring structure.

According to some exemplary embodiments, if system was inserted properly then processor 250 starts to receive heart activity signals at 440, from one or more heart activity sensors that are placed for example in one or more of the coronary sinus, one or both atria and/or one or both ventricles. Optionally, a heart activity sensor is placed at a close proximity to signal conduction pathways, for example the AV node and/or AV node input pathways, for example AV node extensions.

According to some exemplary embodiments, heart activity signals are analyzed and compared to pre-determined parameters stored in the storage component of the system at 441.

Optionally, based on such comparisons, the current clinical state of the patient is determined at 442. According to some embodiments, a decision whether to apply an electric field by the system is automatically made at 443. According to some embodiments, the decision is based for example, on the current clinical state of the patient, the status of the system, and the availability of an application protocol matching the determined clinical state.

According to some exemplary embodiments, if system 240 determined to apply an electric field, then an application protocol is optionally selected at 444, from a list of application protocols stored in the storage component of the system. In some embodiments, the electric field is applied as sub-threshold electric bursts and/or supra-threshold electric bursts.

Optionally, pulse generator 253 is signaled by processor 250 to initiate generation of pulses at 445, according to parameters determined by the application protocol, and to transfer the pulses to electrodes at 446. Optionally, electrodes are placed within the coronary sinus at locations proximal to signal conduction pathways, for example AV node signal conduction pathways. In some embodiments, electrodes are placed outside of the coronary sinus, proximal or at signal conduction pathways, for example AV node region. According to some exemplary embodiments, electrodes are placed proximal to AV node input pathways, for example AV node extensions. Optionally, casing 259 serves as a return electrode.

At 447, when an electrode is electrified with a pulse, this generates an electric field that interacts with target tissue, for example, tissue at the vicinity of the electrodes, for example the coronary sinus wall.

Optionally, one or more Activity sensors receives heart activity signals at 440 and transfer the signals to processor 250 for re-evaluation of the clinical state post electric field application.

### Exemplary Method for Electrode Selection or Electrode Mapping

In some exemplary embodiments of the invention, an electrode or electrodes to be used for electrification are selected from a set of possible electrodes, for example, using mapping. This may include evaluation of efficacy and/or other parameters of various electrodes (e.g., CS and/or non-CS electrodes).

Reference is now made to FIG. 3B, depicting an exemplary embodiment for electrode selection and/or electrode mapping in addition to other acts which may be the same as described for FIG. 3B. While FIG. 3B shows the mapping being done after device implantation and an initial operation protocol, in some embodiments, the mapping as described below is applied before, for example, at 434. In some cases a same electrode is used for mapping and then for later implantation and stimulation. In other embodiments, an initial mapping at 434 uses a first type of electrode (e.g., to determine patient suitability for treatment) and later mapping is after permanent electrode implantation. Optionally, a CS electrode array can be repositioned after mapping.

In some embodiments, for example, as shown, where mapping is provided after an initial stimulation, the initial stimulation may also be selected as part of a mapping procedure, for example, selecting an electrode at an extreme location (e.g., furthest away or closest to ostium or at a most distal or proximal side of the electrode array) and/or selecting a signal parameter set expected to have an effect or expected to be too low to have an effect (e.g., for searching for optimal signal parameters). This may serve as a starting point for searching in the search space of electrode (single or combinations) and/or signals parameters, vs. effects and side effects.

In general, mapping can include a search pathway through the search space (e.g., by electrode order, random, approach from above/below, hill climbing), a starting point and a rule for stopping and/or a rule for selecting an electrode and/or parameter set based on the result. Thereafter, stimulation is applied, results collected and a further point in the search space optionally tested, if a sufficiently usable point is not found. Optionally, a further optimization (e.g., smaller steps, longer test times, different starting cardiac conditions) are applied for points that meet a first criteria (e.g., of efficacy and/or side effects).

According to some exemplary embodiments, following electric field application, the system's processor is configured to receive heart activity signals from one or more heart activity sensors at 448, and to analyze the resulted effect of the applied electric field on the tissue at 450.

If the resulted effect is not the desired effect according to pre-defined parameters, for example, if atrial and/or ventricle contraction rate is not within a desired range, then system's processor is configured to apply another electric field through a different electrode or set of electrodes at 449. In some embodiments, the electrode of choice can be an electrode located distal to the previous electrode or an electrode which is located in proximity to signal conduction pathways or electrode. As can be appreciated, during implantation it may be desirable to implant an electrode array so anatomically promising areas (e.g., with pathways or input to pathways) are bracketed by electrode positions. Changing electrodes may include selecting an electrode closer to or further way form such an anatomically promising area. Optionally or alternatively, changing electrodes may include selecting an electrode at a different circumferential position, so as to target different tissue.

In some exemplary embodiments of the invention, processor 250 is configured to adjust electric field parameters, for example voltage, current, frequency, type of bursts and any combination hereof.

If the resulted effect is the desired effect, then system's processor is configured to apply a second electric field through the selected electrode at 451.

Alternatively, according to some exemplary embodiments, system's processor is configured to deliver an electric field through a different electrode or set of electrodes, to allow mapping the efficacy of electric field application through at least one other electrode at 452. Optionally, processor 250 determines whether to apply an electric field through a selected electrode or to continue mapping the resulted effect from other electrodes based on pre-determined parameters stored within the storage element of the system.

Optionally, mapping includes stimulating at pairs of electrodes as well. Optionally or alternatively, the mapping procedure is managed by a human operator.

In some exemplary embodiments of the invention, mapping results in multiple “possible” electrodes/parameter combinations. Optionally, in therapy, these combinations are used in alternate and/or if a first combination shows a lowered efficacy, a second combination may be used, optionally automatically, or by a user selecting a pre-programmed such combination, rather than re-performing mapping.

Exemplary Method for Atrial Fibrillation Treatment

According to some exemplary embodiments of the invention, the method and system described herein are intended to treat symptoms of atrial fibrillation and other related arrhythmias, for example atrial flutter, for example a symptom of ventricular arrhythmia caused thereby.

Reference is now made to FIG. 3C, depicting a block diagram of a method using a disclosed system, according to some embodiments thereof. In some exemplary embodiments of the invention, to treat atrial fibrillation, electrodes configured to deliver sub-threshold electric bursts are positioned such that at least their distal ends are positioned at or near the AV node of the subject's heart 300. The electrodes may be a plurality of electrodes and/or a stent positioned at the orifice of the coronary sinus, the stent serving as the distal end of at least one electrode or comprising a plurality of electrodes. Each possibility represents a separate embodiment of the present invention. According to some embodiments, positioning electrodes 300 refers to positioning a stent comprising a plurality of electrodes within the coronary sinus of a subject's heart, such as, but not limited to, system 400 depicted in FIG. 4A.

According to some embodiments, the method further comprises positioning at least one heart-activity sensor on the subject's heart. According to some embodiments, positioning system 400 within the coronary sinus enables placing both the plurality of electrodes and the at least one heart activity sensor.

Next, it is determined whether the subject's heart is undergoing atrial fibrillation and has a ventricular rate above a pre-determined range, such as, but not limited to, about 100 BMP 302. The determining may be performed using the disclosed system's processor, based on input received from at least one heart-activity sensor in the subject's heart. According to some embodiments, the determining may be performed according an Automatic Mode Switching algorithm. If the subject is determined to undergo atrial fibrillation and have a ventricular rate above a pre-determined range, such as, but not limited to, about 100 BMP, the system's processor may then proceed to determine which electrode or combination of electrodes is correctly positioned to deliver sub-threshold electric bursts to the AV node.

In some exemplary embodiments of the invention, to determine if an electrode is correctly positioned, sub-threshold electric burst is induced through at least one electrode 304. Next, the ventricular rate is measured to determine 306 whether the ventricular rate is within a pre-determined range following induction 304. The pre-determined range may be within 60-100 beats per minute. If following determining 306 the measured ventricular rate is within the pre-determined range, sub-threshold electrical bursts are delivered 312 through the at least one electrode used in step 304. If following determining 306 the measured ventricular rate is not within the pre-determined range, sub-threshold electrical burst is induced (308) through another electrode or electrode combination than in induction 304.

Following induction 308 the ventricular rate is determined 310 to measure whether it is within the pre-determined threshold. If following determining 310 the measured ventricular rate is not within the pre-determined range, steps 308 and 310 are repeated until at least one correctly positioned electrode or combination is identified. In some exemplary embodiments of the invention, a correctly positioned electrode or combination of electrodes is an electrode or combination which are able to deliver sub-threshold electric bursts to the subject's AV node such that the subject's ventricular rate reaches the pre-determined range. Determining steps 306 and 310 may be performed a pre-determined time period following inductions 304 and 308, respectively, such as, but not limited to, at least 30 seconds or at least 1 minute, alternatively 2, 3, 4 or 5 minutes. Each possibility represents a separate embodiment of the present invention.

Once a correctly positioned electrode or combination has been identified in determining step 306 or 310, sub-threshold electrical bursts are optionally delivered through those electrodes to the subject's AV node 312. The system's processor determines whether the subject has a ventricular rate above the pre-determined range 314 following and/or during induction 312. Each possibility represents a separate embodiment of the present invention. If the subject is determined to no longer have a ventricular which is higher than the pre-determined range, the delivery of the sub-threshold electric bursts may arrest 316. It is to be noted that even when delivery of sub-threshold bursts arrests, sensing of heart activity may be constantly or periodically performed using at least one heart-activity sensor. Each possibility represents a separate embodiment of the present invention.

Once delivery of sub-threshold electric bursts arrests 316, the method may be resumed following sensing of atrial fibrillation and a ventricular rate higher than the pre-determined threshold (FIG. 3C, dashed line). Delivery of sub-threshold electric bursts may be resumed should the system's processor determine that the subject is undergoing another atrial fibrillation episode based on input from the at least one sensor. According to some embodiments, the procedure of determining an electrode or combination of electrodes which is correctly positioned to provide sub-threshold electric bursts to the AV node may be performed once, and any subsequent delivery of sub-threshold electric burst may be performed through the correctly positioned electrodes.

Exemplary System Having Electrodes Near the AV Node

According to some exemplary embodiments, to affect signal propagation between the atria and ventricles, electrodes are positioned in the vicinity of signal conduction pathways, for example, the AV node and/or AV node input pathways, for example AV node extensions. Optionally electrodes are positioned near the coronary sinus ostium. These electrode locations can allow the delivery of an electric field to the AV node through muscle tissues in addition to or instead of neural tissues.

Reference is now made to FIG. 3D, depicting a system 100 attached to or situated near heart 101 according to some embodiments. Processor 102 and electrical-pulse generator 104 may both be encased in casing 106. Casing 106 may comprise additional elements, such as, but not limited to, a storage element functionally coupled with the processor, a battery and a data transmission element. Electrodes 108, 110, 112, 114, coupled with electrical-pulse generator 104 on their proximal ends, are attached on their distal ends to or near AV node 116. The distal ends of at least part of the electrodes may be positioned near or within coronary sinus 118. According to some embodiments, and electrical-pulse generator 104 may be positioned with the distal ends of electrodes 108, 110, 112, 114, such that the electrodes are attached to the pulse generator without leads, as depicted in FIG. 3D. According to some embodiments, processor 102 and electrical-pulse generator 104 may both be attached to or integrally formed with electrodes 108, 110, 112, 114, such that the entire system is positioned at or near AV node 116. According to some embodiments, electrodes 108, 110, 112, 114, processor 102 and electrical-pulse generator 104 may be substantially encase in casing 106 which may be positioned at or near AV node 116. The proximal ends of sensors 120 and 124 are functionally connected to processor 102. According to some embodiments, sensors 120 and 124 are wirelessly attached to processor 102. Sensor 120 is a ventricular sensor attached at its distal end to the right ventricle 122. Sensor 124 is an atrial sensor attached at its distal end to the right atrium 126. According to some embodiments, sensors 120 and 124 provide input to processor 102, such as atrial or ventricular depolarization rate and/or the atrial or ventricular contraction rate. Processor 102 may determine, based on at least part of the input, whether heart 101 is undergoing atrial fibrillation. In a non-limiting example, sensor 124 may sense frequent depolarization events and/or sensor 120 may sense ventricular tachycardia, thus pointing to atrial fibrillation. According to some embodiments, either one or both sensors 120 and 124 may be situated at or near AV node 116. According to some embodiments, a single sensor may be used to sense ventricular activity and atrial activity and be in place of both sensors 120 and 124.

According to some embodiments, either one or both sensors 120 and 124 may be attached to or integrally formed with at least part of electrodes 108, 110, 112, 114, processor 102 and electrical-pulse generator 104 and/or may be encased in casing 106, the attached/encased system being situated at or near AV node 116. Upon sensing of atrial fibrillation, processor 102 then induces the induction of sub-threshold electric bursts in electrical-pulse generator 104. Prior to the first use, processor 102 may perform a calibration to determine which electrode or combinations thereof are correctly positioned to deliver sub-threshold electric bursts to AV node 116. According to some embodiments, the calibration may be repeated every pre-determined time period, such as, but not limited to, every 24 hours, every 7 days or every 30 days. Each possibility represents a separate embodiment of the present invention.

It is a particular feature of some embodiments of the invention, that atrial activity and/or ventricular activity are sensed from within a CS, e.g., using one, two or more electrodes located within the CS.

In order for processor 102 to calibrate and determine which electrode is correctly positioned, the processor may first direct the sub-threshold electric bursts through electrode 108. Following direction of the electric bursts through electrode 108, processor 102 optionally measures the ventricular rate through input received from sensors, such as, but not limited to, ventricular sensor 120. Such sensing may be for a pre-determined time period, such as, but not limited to at least 30 seconds or at least one minute. Each possibility represents a separate embodiment of the present invention.

If following delivery of electric bursts through electrode 108 the ventricular rate is within a pre-determined range, such as, but not limited to 60-100 beats per minute, processor 102 may determine that electrode 108 is correctly positioned. If following delivery of electric bursts through electrode 108 the ventricular rate is not within the pre-determined range, processor 102 may repeat the inducing/sensing with another electrode, such as electrode 110 or a combination of electrodes, such as, but not limited to electrodes 112 and 114 until an electrode which is correctly positioned is identified. Processor 102 may then induce delivery of sub-threshold electric bursts to AV node 116 through the correctly positioned electrodes in intervals which enable maintenance of ventricular rate within the pre-determined range. According to some embodiments, if processor 102 senses through a sensor such as atrial sensor 124 that heart 101 is not undergoing atrial fibrillation, the delivery of sub-threshold electrical bursts is optionally stopped until another episode of atrial fibrillation is sensed.

Exemplary System Having Electrodes within the Coronary Sinus

In some embodiments, one of the preferred locations for placing electrodes is within the coronary sinus, near the coronary sinus ostium. As shown previously in FIG. 1B, the coronary sinus ostium 30 is found in close proximity to AV node, and right inferior AV node extensions 25, 36. According to some exemplary embodiments, system electrodes can be coupled to an anchoring element configured to be placed within the coronary sinus.

Reference is now made to FIG. 3E, depicting system 200 attached to or situated near heart 201 according to some embodiments. Casing 206 comprises processor 202 and electrical-pulse generator 204. Stent 208 serves as the distal end of electrode 210 which is functionally connected to electrical-pulse generator 204 on its proximal end. Alternatively, stent 208 may include the distal ends of several electrodes which are functionally connected to electrical-pulse generator 204 on their proximal ends. According to some embodiments, stent 208 may include a plurality of electrodes or the distal ends thereof, each electrode functionally connected to electric-pulse generator 204 or each electrode functionally connected to at least one electric-pulse generator and leadlessly connected to processor 202. Each possibility represents a separate embodiment of the present invention. System 200 may further comprise non-stent like electrodes which have distal ends positioned at or near AV node 216.

Stent 208 is positioned within the orifice of coronary sinus 218 which is positioned proximally to AV node 216. According to some embodiments, processor 202 may induce delivery of sub-threshold electrical bursts to AV node 216 through stent 208 or electrodes embedded within it. According to some embodiments, processor 202 is configured to induce delivery of sub-threshold electric bursts only if at least one of sensors 220 and 224 senses that heart 201 undergoes atrial fibrillation and that the ventricular rate is above a pre-determined range, such as, but not limited to, above 100 BPM. Sensor 220 is a ventricular sensor attached at its distal end to the right ventricle 222. Sensor 224 is an atrial sensor attached at its distal end to the right atrium 226.

According to some embodiments, system 202 in its entirety is comprised in or at least partly attached to or integrally formed with stent 208. Each possibility represents a separate embodiment of the present invention. According to some embodiments, all elements of system 202 are comprised in or at least partly attached to or integrally formed with stent 208 such that the system may be inserted to the coronary of a sinus as a single lead-less unit. Each possibility represents a separate embodiment of the present invention.

According to some embodiments, all elements of system 202 are comprised in or at least partly attached to or integrally formed with stent 208 such that the system may be inserted to the coronary of a sinus as a single lead-less unit, wherein system 202 further comprises an energy source such as a battery attached to or at least partially integrally formed with stent 208 such that an external energy source is not required. According to some embodiments, stent 208 comprises a plurality of electrodes, at least one electrical-pulse generator such as electric pulse-generator 204 which is functionally connected to the electrodes, processor 202 functionally connected to the pulse-generators and/or the electrodes and at least one heart-activity sensor such as sensors 220 and 224 able to sense ventricular and/or atrial activity. Each possibility represents a separate embodiment of the present invention.

Exemplary Electrodes Position within the Coronary Sinus

According to some exemplary embodiments electrodes are configured to be inserted into the coronary sinus and to be positioned in close proximity to the AV node.

Reference is now made to FIGS. 3F and 3G, schematically depicting electrodes position within the coronary sinus, according to some embodiments of the invention. According to some exemplary embodiments, electrodes 112 and 114 are configured to be inserted into coronary sinus and to be located near coronary sinus ostium 30. According to some exemplary embodiments, system's processor 250 is configured to select a preferred electrode for electric field application, based on its proximity to AV node 25 or AV node extensions. For clarity, AV node 25 is shown above the CS and spaced away from the ostium. According to some exemplary embodiments, system's processor is configured to select a preferred electrode for electric field application, based on its ability to affect signal propagation through AV node 25.

According to some exemplary embodiments, electrodes are attached to or manufactured with a support element, for example stent 208 configured to anchor electrodes within the coronary sinus. According to some exemplary embodiments, stent 208 is inserted at least partly within coronary sinus 218, and is configured to anchor electrodes near coronary sinus ostium 30, near AV node 25.

Exemplary Leadless System

According to some exemplary embodiments, the electrification system is configured to be leadless, with no lead wiring between its components.

Reference is now made to FIG. 4A, depicting leadless system 400 according to some embodiments of the invention. According to some embodiments, system 400 is configured to be entirely inserted into the coronary sinus of a subject's heart. System 400 comprises stent 418 in the distal end of the system and stent 402 in the proximal end of the system. Stent 402 is configured to be positioned closer to the proximal end of the coronary sinus, the end of the coronary sinus which opens to the right atrium. Stent 418 is configured to be positioned closer to the distal end of the coronary sinus, this may be a better location for anchoring by radial expansion. According to some embodiments, stents 402 and/or 418 comprise at least one electrode and/or at least one heart-activity sensor. Each possibility represents a separate embodiment of the present invention. According to some embodiments, stent 402 comprises a plurality of electrodes configured to deliver sub-threshold electric bursts to the subject's AV node. FIG. 4B depicts a part of stent 402, according to some embodiments, comprising a plurality of electrodes such as electrodes 402A, 402B and 402C. According to some embodiments, stent 402 is comprised of a mesh of a material such as, but not limited to, nitinol.

According to some embodiments, electrodes 402A, 402B and 402C are situated at the junctions of the mesh, as exemplified in FIG. 4B. According to some embodiments, stent 402 comprises at least one heart-activity sensor. According to some embodiments, at least some of the electrodes of stent 402, such as, but not limited to, electrodes 402A, 402B and 402C may serve as a heart-activity sensor. According to some embodiments, stent (418) comprises at least one heart-activity sensor.

According to some embodiments, stents 402 and/or 418 are configured to have an open conformation and a closed conformation. Each possibility represents a separate embodiment of the present invention. According to some embodiments, upon insertion of system 400 to the coronary sinus of the subject's heart, stents 402 and/or 418 are in the closed conformation. According to some embodiments, stents 402 and 418 are transferred from the closed conformation to the open conformation concurrently or sequentially after system 400 has been fully inserted into the coronary sinus. According to some embodiments, when stents 402 and 418 are in the open conformation they are able to fix the system to a desired location within the coronary sinus, such as a location which enables delivery of sub-threshold electric bursts by the system to the subject's AV-node.

According to some embodiments, stents 402 and 418 are attached via flexible tube 404. According to some embodiments, flexible tube 404 is configured to be flexible such that it is able to conform to the outline of the coronary sinus. According to some embodiments, the girth of flexible tube 404 is configured to enable blood flow within the coronary sinus. According to some embodiments, at least some of the elements of system 400 are comprised in and/or are attached to flexible tube 404. According to some embodiments, stents 402 and 418 are functionally connected to and/or are integrally formed with flexible tube 404 and/or the elements of system 400 comprised within flexible tube 404. According to some embodiments, flexible tube 404 may be comprised of a flexible material, such as, but not limited to, a flexible polymer.

According to some embodiments, leadless system 400 comprises at least one processor, such as processors 414 and 416, at least one energy source such as batteries 410 and 412 and at least one electric pulse generator such as electric pulse generators 406 and 408. According to some embodiments, processors 414 and 416 and/or batteries 410 and 412 and/or electric pulse generators 406 and 408 are comprised within flexible tube 404. According to some embodiments, batteries 410 and 412 provide sufficient energy to system 400 such that an additional energy source is not required.

According to some embodiments, system 400 is configured to be inserted into the subject's coronary sinus via an insertion device, such as, but not limited to, a catheter. According to some embodiments, stent 418 is inserted to the coronary sinus followed by tube 404 and stent 402. System 400 may be inserted such that both stents 402 and 418 are in a closed conformation, thus enabling positioning the system in the desired location within the coronary sinus. Once system 400 is inserted into the desired location within the coronary sinus stents 402 and 418 may be switched to their open conformation, thus fixing system 400 to the desired location within the coronary sinus.

Exemplary Leadless System within the Coronary Sinus

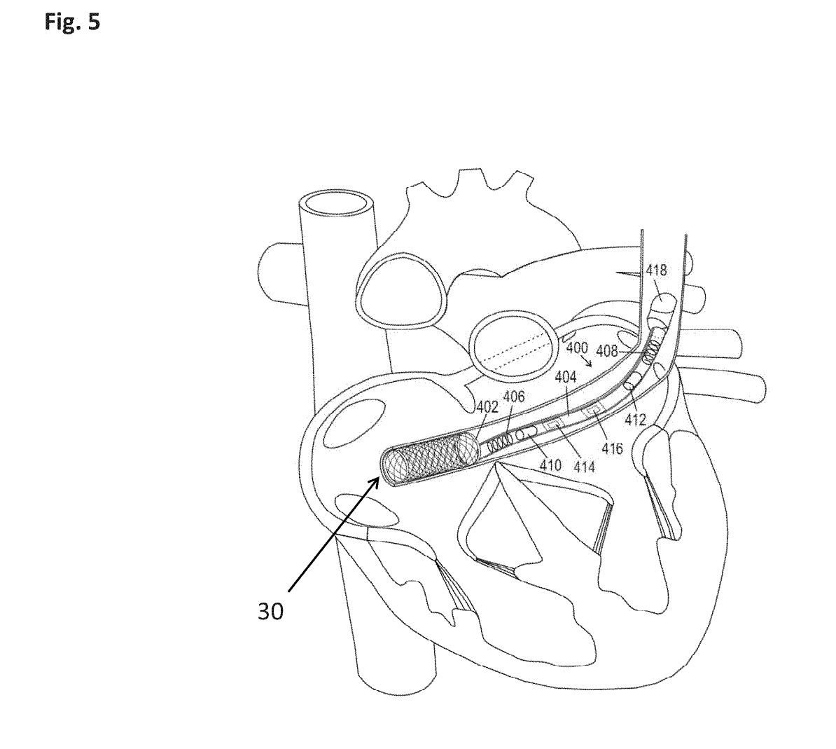

FIG. 5 shows system 400 inserted at least partly within the coronary sinus, in accordance with some embodiments of the invention. According to some exemplary embodiments, system 400 is inserted at least partially within the coronary sinus, with stent 402 having electrodes at its outer surface, is placed near the coronary sinus ostium 30. According to some embodiments, upon insertion of system 400 into a predetermined location within the coronary sinus, for example, as discuss in FIG. 3A, stent 402 and/or stent 418 are configured to expand and to anchor system 400 by applying pressure against the inner surface of coronary sinus wall. According to some exemplary embodiments, stents 402 and 418 are configured to allow the flow of fluids, for example blood when system 400 is inserted into the coronary sinus. Optionally, system 400 blocks less 20%, 50%, 80%, 90% or intermediate percentages of the flow. According to some embodiments, when stent 402 expands, at least some electrodes of stent 402 are configured to be in contact with the coronary sinus wall. According to some exemplary embodiments, if system 400 and/or stent 402 are not placed within a predetermined location, then system 400 is configured to move and/or rotate within the coronary sinus to adjust its orientation. According to some exemplary embodiments, when system 400 is anchored at least partially within the coronary sinus, at least one heart activity sensor is in contact with coronary sinus wall and/or with tissue near the coronary sinus.

Exemplary Support Elements Comprising Electrodes

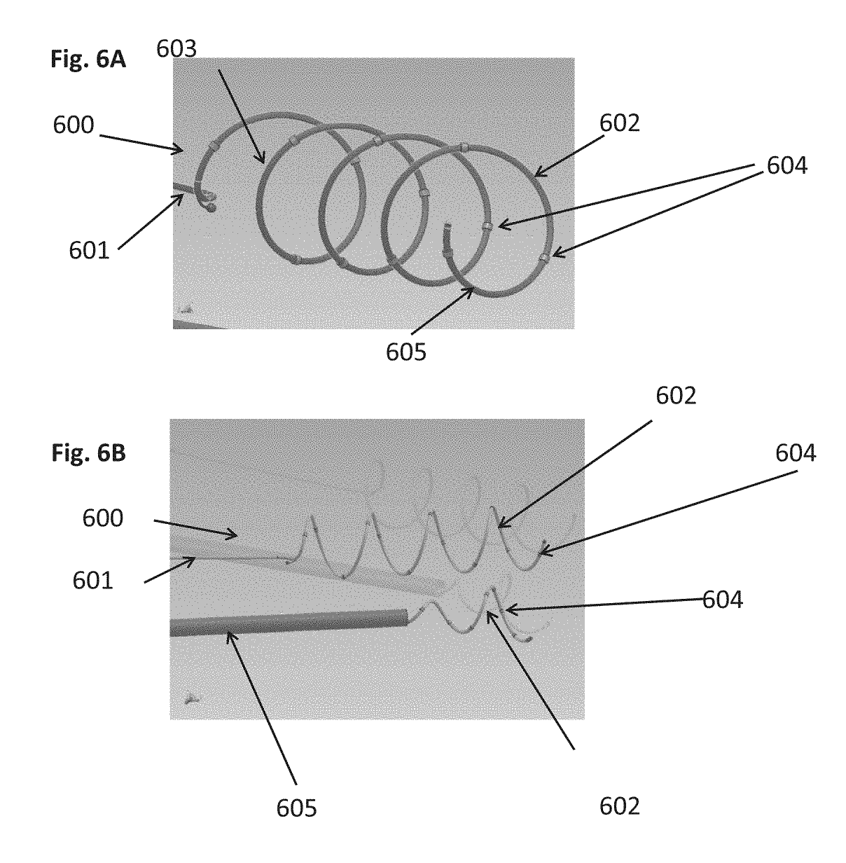

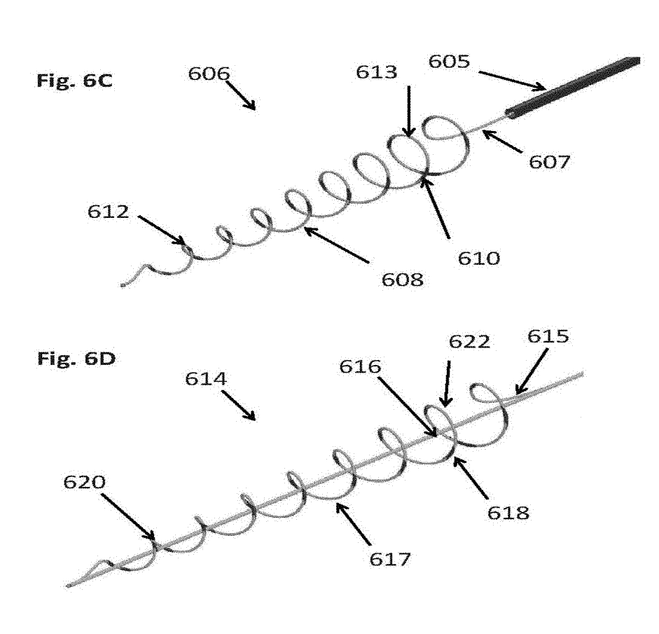

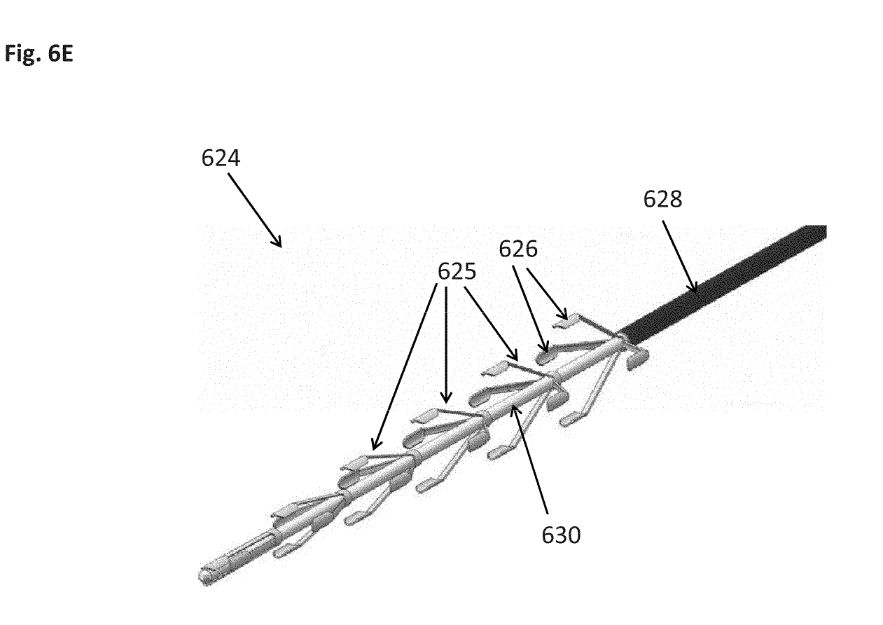

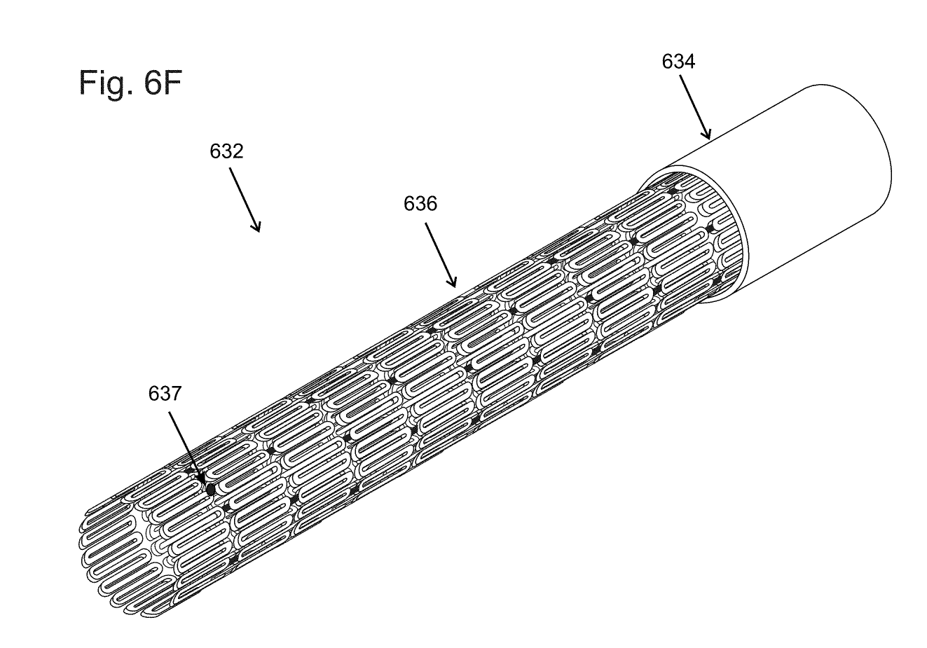

According to some exemplary embodiments, placing electrodes within a blood vessel, for example coronary sinus is facilitated by providing a combined anchoring structure with electrodes thereon. Optionally, anchoring ensures contact between an electrode and the local anchoring tissue and/or prevents relative movement (e.g., for non-contact electrification) Reference is now made to FIGS. 6A-6F, depicting exemplary embodiments of electrodes combined with a support element, configured to be part of the disclosed system.

FIGS. 6A and 6B depict a combined structure 600 comprising a helical support element 602 with a plurality of electrodes 604 provided thereon. According to some exemplary embodiments, combined structure 600 is configured to be inserted into a blood vessel, for example coronary sinus, and to anchor electrodes within said blood vessel. According to some embodiments, helical support element 602 is formed by bending a wire or tube 601 into a helical conformation, having a proximal 603 and distal 605 endings with, for example an equal diameter. In some embodiments, the diameter of a central part of the helix is not equal and/or the diameters at the ends are not equal; FIG. 6C showing an example where one end has a substantially zero diameter.

According to some exemplary embodiments, helical support element 602 comprises electrodes 604 evenly or non-evenly distributed along the helix. According to some exemplary embodiments, wire 601 and helical structure 602 are formed from a shape memory material, for example Nitinol.

According to some exemplary embodiments, combined structure 600 is encased within a flexible tube 605 during insertion into blood vessel. When tube 605 reaches a desired location, it is retracted, while holding structure 600 in place, and allows combined structure 600 to self expand. According to some exemplary embodiments, expansion of combined element 600 applies pressure against blood vessel walls, and attaches at least some of electrodes 604 to blood vessel walls. FIG. 6B shows various stages during such expansion and/or compression (e.g., if sheath 605 is advanced over structure 600).

FIG. 6C depicts a combined structure 606 which can be the same or similar to that shown in FIGS. 6A and/or 6B, however, the diameter of structure 606 (e.g., of a helical support element 608 with one or more electrodes 610 thereon) decreases in a distal direction (e.g., from a proximal end 613 to a distal end 612). this may provide a better fit to the CS geometry. Optionally, element 608 is hollow and carried power leads to electrodes 610.

Optionally, helical support element 608 comprises electrodes 610 distributed evenly along the helix and/or an electrode at a distal tip thereof. According to some exemplary embodiments, wire 607 and helical support element 608 are formed from a shape memory material, for example Nitinol.

According to some exemplary embodiments, combined structure 606 is encased within a flexible tube or sheath 605 during insertion into blood vessel. During system insertion, when tube 605 reaches a desired location, tube 605 is retracted, and allows combined element 606 to expand. According to some exemplary embodiments, expansion of combined element 606 applies pressure against blood vessel walls, and attach at least some of electrodes 610 to blood vessel walls. Advancing of sheath 605 is optionally used to radially compress and de-anchor structure 606.

FIG. 6D depicts a combined structure 614 which can be the same or similar to that shown in FIG. 6C (or 6A) and which includes a central shaft 616. Optionally, shaft 616 provides axial stability (e.g., to reduce relative movement of coils and/or improves anchorability due to an added resilience to bending. While as shown, shaft 616 is only attached at a distal end 620 and/or a proximal end 615, in some embodiments, shaft 616 is attached at one or more locations along the length of structure 614, for example, at each or at every other winding.

FIG. 6E depicts a combined structure 624 which includes an array of separate electrodes. In some exemplary embodiments of the invention, the array comprises subsets of electrodes each attached to a central shaft 630, at different axial locations thereof. While the figure shows each such subset as including plurality of electrodes 626, each with its own rib-like support 625, this need not be the case. For example, one or more axial locations may include a ring electrode. Optionally or alternatively, one electrode may have two or more rib-supports. Optionally or alternatively, one rib support may support 2 or more electrodes.

In some exemplary embodiments of the invention, the ribs are predisposed to have a general conical geometry, so that electrodes of each subset have different resting radial distances away from shaft 630 (e.g., to match an expected CS geometry). Optionally, different electrodes at a same axial location also have different distances.

In some exemplary embodiments of the invention, each electrode 626 is designed to have a local, directional effect and/or to be curved and/or otherwise atraumatic to CS wall tissue. Optionally, however, pressure applied by supports 625 and electrodes 626 is sufficient to anchor electrodes 626 in place, at least temporarily. In some exemplary embodiments of the invention, an additional expanding structure (such as a stent-like cylinder) is provided for anchoring.

In some exemplary embodiments of the invention, deployment is by self expanding of supports 625 when an encasing sheath 628 is retracted. Optionally or alternatively, removal is by advancing sheath 628 to collapse supports 625.

In some embodiments, a plurality of supports 625 are attached to each other at a ring which is mounted on shaft 630. In embodiments, the ribs of a subset of electrodes are not all attached to a same ring, have a same extension profile and/or result in electrodes at a same axial position.

FIG. 6F depicts a combined structure 632 comprising a cylindrical generally grid-shaped support element 636 (e.g., using a stent-strut design). According to some embodiments, cylindrical grid-shaped support element 636 comprising at least one electrode 637 distributed along the support element. According to some embodiments, electrodes are spaced apart along support element 636, with a minimal distance of 1 mm between every two adjacent electrodes and/or a maximum of 6 mm between neighboring electrodes. Optionally, at least one electrode is a ring-shaped electrode. Optionally or alternatively, at least one electrode is a flat electrode covering less than 40% of a circumference of structure 636. Optionally, each electrode has its own electrification wire (not shown) optionally found within the lumen of 636 or trapped between the surface of 636 and the CS wall.

According to some exemplary embodiments, grid-shaped support element 636 is encased within tube 634 during insertion of combined structure 632 into blood vessel, for example coronary sinus. According to some embodiments, when reaching a desired location within the CS, tube 634 is retracted and allows grid-shaped support element to self expand and apply pressure against blood vessel wall. Optionally or alternatively, balloon expansion is used. In either case, structure 632 may be delivered on a guide wire.

Optionally, an edge of the structure is configured to be flared to contact the CS ostium and/or atrial tissue surrounding the ostium. Optionally, such sections include at least 1, 2, 3 electrodes or more.

According to some embodiments, the present disclosure provides an implantable electrical stimulation system for providing atrial fibrillation therapy to a subject, the system comprising:

at least one electrical-pulse generator configured to generate sub-threshold electric bursts;
at least one heart-activity sensor;
at least one electrode functionally connected to said at least one electrical pulse generator and configured to deliver said sub-threshold electric bursts to the subject's atrioventricular (AV) node;
a processor configured to receive input from said at least one heart-activity sensor; measure the ventricular rate of said subject using at least part of said input; and determine whether said subject is undergoing atrial fibrillation based on at least part of said input;
induce delivery of sub-threshold electric bursts to the subject's AV node through said at least one electrode if said subject is undergoing atrial fibrillation and has a ventricular rate above a pre-determined range;
a stent configured to be inserted into the coronary sinus of the subject's heart, wherein said stent comprises or is attached to or is integrally formed with at least one of: said at least one electrical-pulse generator, said at least one heart-activity sensor,
said at least one electrode, said processor and a combination thereof. Each possibility represents a separate embodiment of the present invention.

According to some embodiments, the present disclosure provides an implantable electrical stimulation system for providing atrial fibrillation therapy to a subject, the system comprising:

at least one electrical-pulse generator configured to generate sub-threshold electric bursts;
at least one heart-activity sensor;
at least one electrode functionally connected to said at least one electrical pulse generator and configured to deliver said sub-threshold electric bursts to the parasympathetic ganglion plexi of the subject's heart;
a processor configured to:
receive input from said at least one heart-activity sensor;
measure the ventricular rate of said subject using at least part of said input;
determine whether said subject is undergoing atrial fibrillation based on at least part of said input;
induce delivery of sub-threshold electric bursts to the parasympathetic ganglion plexi of the subject's heart through said at least one electrode if said subject is undergoing atrial fibrillation and has a ventricular rate above a pre-determined range; and

a stent configured to be inserted into the coronary sinus of the subject's heart, wherein said stent comprises or is attached to or is integrally formed with at least one of: said at least one electrical-pulse generator, said at least one heart-activity sensor, said at least one electrode, said processor and a combination thereof. Each possibility represents a separate embodiment of the present invention.

According to some embodiments, the system is a leadless system. According to some embodiments, the at least one electrode is a plurality of electrodes. According to some embodiments, the processor is further configured to determine which subset of the plurality of electrodes is able to deliver sub-threshold electric bursts to the subject's AV node such that said bursts induce a ventricular rate within a pre-determined range; and actuate delivery of sub-threshold electric bursts through said subset.

### General

It is expected that during the life of a patent maturing from this application many relevant sub-threshold signals will be developed; the scope of the term sub-threshold is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers there between.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating one or more clinical and/or aesthetical symptoms of a condition and/or substantially preventing the appearance of one or more clinical and/or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. (canceled)

2. Apparatus for cardiac electrification, comprising:
(a) at least one electrode sized and shaped for placement within an adult coronary sinus of a heart;
(b) a signal generator electrically coupled to said electrode and configured to electrify said electrode so that said electrode applies a sub-threshold electric field through a wall of said coronary sinus to an atrial-ventricular conduction pathway or associated tissue and wherein said signal generator is configured to generate said sub-threshold electric field with parameter values selected to modify conduction of an atrial activation through the pathway to activate a ventricle.

3. Apparatus according to claim 2, wherein said at least one electrode is mounted on a structure sized and shaped for anchoring in said adult coronary sinus.

4. Apparatus according to claim 3, wherein said structure is sized and pre-deformed to self-expand to anchor in said coronary sinus.

5. Apparatus according to claim 3, wherein said structure is sized and pre-deformed to self-expand to put said at least one electrode in contact with said wall of said adult coronary sinus in a selected location on said wall suitable for delivery of said sub-threshold electric field to block said conduction.

6. Apparatus according to claim 2, wherein at least one of said at least one electrode is sized and shaped for positioning wholly within 7 mm of an ostium of said coronary sinus.

7. Apparatus according to claim 2, comprising at least one electrode selectively located on said apparatus for positioning said electrode wholly outside the CS.

8. Apparatus according to claim 2, wherein said pathway comprises an AV node.

9. Apparatus according to claim 8, wherein said at least one electrode and electrification electrify tissue which acts as input to said AV node.

10. Apparatus according to claim 2, wherein said at least one electrode and electrification modify conduction, at least mostly, by the action of said field on muscle fibers.

11. Apparatus according to claim 2, wherein said signal generator generates said sub-threshold electric field with parameter values selected to block conduction of at least 20% of activations passing through said pathway, from reaching said ventricle with an amplitude and timing sufficient to activate said ventricle.

12. Apparatus according to claim 2, wherein said signal generator generates said sub-threshold electric field with parameter values selected to create temporal windows in the activity of the pathway within which an activation from the atria is more likely to reach a ventricle than outside the window.

13. Apparatus according to claim 2, wherein said parameters values of said sub-threshold electric field comprise electric field current of 0.1-5 mA and/or electric field voltage of 0.05-15.

14. A method for modifying electrical activity in the heart, comprising:
(a) applying a sub-threshold electric field to an AV node and/or associated tissue using at least one electrode located within a coronary sinus, wherein said sub-threshold electric field is applied through a wall of said coronary sinus to said AV node and/or associated tissue and wherein said sub-threshold electric field is applied with parameters values selected to block conduction of an atrial activation through the AV node to activate a ventricle; and
(b) blocking conduction of atrial activation through said AV node to a ventricle by said applying.

15. A method according to claim 14, wherein said blocking comprises blocking conduction of at least 20% of activations passing through said AV node from reaching said ventricle with an amplitude and timing sufficient to activate said ventricle, by said applying.

16. A method according to claim 14, wherein said applying comprises generating a temporal window within which activation passage from said atria to said ventricle is better than outside the window.

17. A method according to claim 14, wherein said applied sub-threshold electric field is sub-threshold to said AV node and/or associated tissue in that it does not generate a new propagating action potential, which can propagate further than 5 mm, therein.

18. A method according to claim 14, comprising positioning said at least one electrode within 5 mm of an ostium of said coronary sinus.

19. A method according to claim 14, comprising measuring heart activity following said applying by at least one sensor, and adjusting said parameter values of said sub-threshold electric field based on the results of said measuring.

20. A method according to claim 14, comprising positioning at least two electrodes within said coronary sinus, and wherein said applying comprises applying said sub-threshold electric field by at least one electrode of said at least two electrodes.

21. A method according to claim 20, comprising measuring heart activity following said applying by at least one sensor, and selecting a different electrode from said at least two electrodes for said applying based on the results of said measuring.
